(19) United States
(12) Patent Application Publication
(10) Pub. No.: US 2025/0026733 A1
(43) Pub. Date: Jan. 23, 2025

(54) DEUTERATED ANALOGS OF PYROLE INHIBITORS OF ERK, SYNTHESIS THEREOF AND INTERMEDIATES THERETO

(71) Applicant: BIOMED VALLEY DISCOVERIES, INC., Kansas City, MO (US)

(72) Inventors: Gary DECRESCENZO, Parkville, MO (US); Martin TERESK, Parkville, MO (US)

(21) Appl. No.: 18/893,686

(22) Filed: Sep. 23, 2024

Related U.S. Application Data
(63) Continuation of application No. PCT/US23/64908, filed on Mar. 24, 2023.
(60) Provisional application No. 63/323,221, filed on Mar. 24, 2022.

(51) Int. Cl. C07D 401/04; A61K 31/4439; A61P 35/00; C07B 59/00

(52) U.S. Cl. CPC C07D 401/04; A61K 31/4439; A61P 35/00; C07B 59/002

(57) ABSTRACT

The present disclosure provides, inter alia, deuterated analogs of ulixertinib and pharmaceutically acceptable salts thereof, which are effective inhibitors of ERK protein kinases and have surprisingly improved pharmacokinetic and metabolite formation properties. The present disclosure also provides, inter alia, methods of making and using same. Kits containing compounds and pharmaceutical compositions of such deuterated analogs are also provided.

### CROSS REFERENCE TO RELATED APPLICATIONS

The present application claims benefit of U.S. Provisional Patent Application Ser. No. 63/323,221, filed on Mar. 24, 2022, which application is incorporated by reference herein in its entirety.

### BACKGROUND

The absorption, distribution, metabolism and excretion (ADME) properties of drugs are critical characteristics that can mean the difference between a safe/effective drug and a clinical and commercial failure. Recent advances in drug formulation technologies (and drug conjugates or prodrugs) have offered some ability to improve ADME properties in limited cases, but underlying ADME problems are still a major cause of the failure of drugs in clinical trials. A common ADME issue with currently approved drugs and drug candidates is rapid metabolism. A drug candidate that otherwise is highly efficacious in in vitro and preclinical testing, can be metabolized too quickly and cleared from the body giving little to no pharmacological effect. Strategies to overcome fast metabolism include dosing at very high levels or dosing very frequently, but both strategies have significant drawbacks, including increasing the side effects of drugs, increasing exposure to toxic metabolites, and decreasing patient dosing compliance due to frequency.

In some cases, metabolic inhibitors have been used to improve the characteristics of a particular drug (see Kempf, D. et al. Antimicrobial Agents and Chemotherapy, 41(3), p. 654 (1997); Wang, L. et al. Clinical Pharmacology and Therapeutics, 56(6 Pt. 1), p. 659 (1994). This strategy, however, is not widely used because it can lead to serious unwanted side effects, and undesired drug-drug interactions. Optimization of drug structure usually involves an iterative process of structure modification to improve biological activity and/or metabolic properties. A better metabolic profile, however, often comes at the expense of biological potency and efficacy because of the significant modifications of a desired pharmacophore structure needed to stop or slow the biological degradation processes.

One possible strategy for improving the metabolic profile of a drug, without substantially altering the biological potency and efficacy, is to substitute one or more hydrogen atoms with deuterium to slow cytochrome P₄₅₀ mediated metabolism. Cytochrome P₄₅₀ enzymes are a family of enzymes capable of catalyzing oxidative and reductive biotransformation of most drugs and are a major source of variability in drug pharmacokinetics and patient response to treatment. Deuterium is an isotope of hydrogen that contains an additional neutron in its nucleus, and is safe, stable and nonradioactive. Due to the increased mass of deuterium as compared to hydrogen, the bond between carbon and deuterium requires greater energy to cleave as compared to the bond between hydrogen and carbon, which can reduce metabolic reaction rates. Specifically, the activation energy required for reaching the transition state for bond cleavage is greater for a carbon-deuterium bond compared to a carbon-hydrogen bond and, therefore, the reaction rate is slower. The reduced metabolic reaction rate can favorably impact a molecule's ADME properties, giving improved efficacy, safety, and tolerability. Other physical characteristics of deuterium are essentially identical to hydrogen and would not be expected to have a biologically relevant effect on a molecule with deuterium replacement.

A small number of drugs have been tested that employ deuterium substitution to improve metabolism (see Blake, M. et al. J. Pharm. Sci., 64, p. 367 (1975); Foster, A. Adv. Drug Res., 14, p. 1 (1985); Kushner, D. et al. Can. J. Physiol. Pharmacol., p. 79 (1999); Fisher M. et al. Curt. Opin. Drug Discov. Devel., 9, p. 101 (2006)). The effect of deuterium replacement of hydrogen on metabolic rate, however, has proven not to be predictable and has led to variable results. In some cases, deuterated compounds had a decreased metabolic clearance in vivo, while for others, there was no change in metabolic clearance, and yet others unexpectedly showed an increase in metabolic clearance. This unpredictability of ADME is a significant challenge for deuterium replacement as a strategic drug design modification for reducing metabolic rate (see Foster and Fisher, supra).

Even when a site and position of metabolism is known, deuterium replacement does not have a predictable effect on metabolic rate. It is only by preparation of the specific deuterium substituted drug (candidate) and testing that one can determine the extent of change in metabolic rate. See Fukuto, J. et al. J. Med. Chem., 34(9), p. 2871 (1991). Many, if not most, drug candidates have multiple sites where metabolism is possible, with the metabolic site profile being unique to each drug molecule. Thus, deuterium replacement requires anew study for metabolic effect for each drug candidate. See Harbeson, L. and Tung. R. Medchem News, 2, p. 8 (2014) and references therein. There are several examples of drug candidates where deuterium substitution of hydrogen has led to an enhanced metabolic rate and/or metabolic switching, or no in vivo change of the molecule's metabolic profile even after metabolic slowing. Harbeson et al. reveal that selective deuteration of paroxetine at predicted metabolically labile positions actually produced analogs which demonstrated increased metabolism in vivo (Scott L. Harbeson and Roger D. Tung, Deuterium in Drug Discovery and Development, 46 annual report in medicinal chemistry, 403-417 (2011)). Furthermore, Miwa reports that deuteration of metabolically labile sites may lead to the potentiation (or switching) of alternative metabolic pathways, with then undetermined consequences (Miwa. G., Lu, A., Kinetic Isotope Effects and ‘Metabolic Switching’ in Cytochrome P450-Catalyzed Reactions, 7 Bioassays, 215-19 (1987)). For example, phentermine has been deuterated to decrease its metabolic rate, however replacement of N,N-dimethyl hydrogens with deuterium resulted in no change observed (Allan B. Foster, “Deuterium Isotope Effects in the Metabolism of Drugs and Xenobiotics: Implications for Drug Design”, Advances in Drug Research, (14), 1-40 (1985)). Similarly, deuteration of metabolically active sites of tramadol led to no increase in duration of effect (Shao et. al., “Derivatives of Tramadol for Increased Duration of Effect”, Bioorganic and Medicinal Chemistry Letters, (16), 691-94 (2006)).

Ulixertinib (BVD-523) was originally disclosed in U.S. Pat. No. 7,354,939, by Martinez-Botella et al. (which is incorporated by reference in its entirety), and has been shown, e.g., to be a potent inhibitor of ERK1/2 making it a promising therapeutic for treatment of cancers and other diseases. When administered in vivo, however, ulixertinib can be metabolized to result in the formation of at least six metabolites, some of which may be undesirable, and is easily cleared. (See, Bin Yu et al., “Pharmacokinetics and metabolism of ulixertinib in rat by liquid chromatography combined with electrospray ionization tandem mass spectrometry,” Separation Science, vol. 43, issue 7, pages 1275-1283 (2020)) Thus, there exists a need for analogs of ulixertinib to reduce or slow the formation of metabolites, and to reduce systemic and pre-systemic clearance. The present disclosure is directed to meeting these and other needs.

### SUMMARY OF THE DISCLOSURE

According to some aspects, the present disclosure provides novel compounds that are ulixertinib derivatives, and pharmaceutically acceptable salts thereof, that are effective as inhibitors of ERK protein kinase. In some embodiments, these compounds have the general Formula 1:

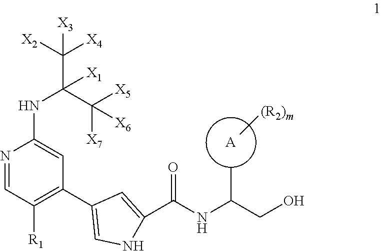

including pharmaceutically acceptable salts, solvates, and prodrugs thereof, wherein each X₁, X₂, X₃, X₄, X₅, X₆, and X₇ are independently selected from the group consisting of hydrogen and deuterium or C₁₋₄ aliphatic, and wherein each of Ring A, R₁, R₂, and m are as defined as disclosed herein.

In some aspects, the present disclosure also provides compositions comprising a compound as disclosed herein and the use of such compounds in methods for treating or lessening the severity of a variety of disorders, including proliferative disorders such as cancer.

In some embodiments, the selective replacement of hydrogen atoms with deuterium in the compounds disclosed herein offers the unique benefit of retaining the physicochemical properties and pharmacological profile of the parent compound while positively impacting its metabolic fate by reducing or slowing the formation of an undesired metabolite, reducing systemic clearance of the compound, thus, increasing its half-life, and decreasing pre-systemic metabolism resulting in higher bioavailability of the unmetabolized compound, which can, in principle, improve the safety, efficacy, and/or tolerability of the compound. Furthermore, in some embodiments, deuterium substitution in the compounds disclosed herein also decreases inhibition and or induction of at least one cytochrome P450 metabolic enzyme as compared to the non-isotopically enriched compound, which consequently reduces the risk of drug-drug interactions.

According to some aspects, the present disclosure provides compounds of Formula 1:

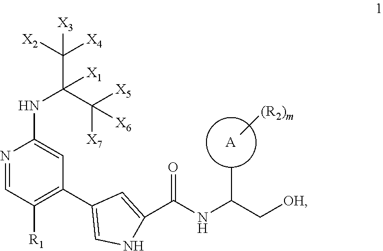

or a pharmaceutically acceptable salt, solvate, or prodrug thereof, wherein each of X₁, X₂, X₃, X₄, X₅, X₆, and X₇ are independently selected from the group consisting of hydrogen, deuterium, and C₁₋₄ aliphatic, and wherein: R₁ is hydrogen, C₁₋₃ aliphatic, fluoro, or chloro; Ring A is an optionally substituted group selected from phenyl, 5-6 membered monocyclic heteroaryl ring having 1-5 heteroatoms independently selected from nitrogen, oxygen, or sulfur, or a 5-6 membered saturated or partially unsaturated heterocyclic ring having 1-5 heteroatoms independently selected from nitrogen, oxygen, or sulfur; R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; and m is 0, 1, or 2.

According to some embodiments, the present disclosure provides a compound of Formulas:

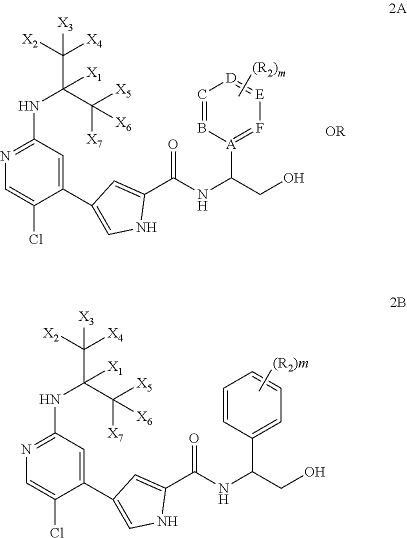

or a pharmaceutically acceptable salt, solvate, or prodrug thereof, wherein: each X₁, X₂, X₃, X₄, X₅, X₆, and X₇ are independently selected from hydrogen and deuterium or C₁₋₄ aliphatic; R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; m is 0, 1, or 2; and A, B, C, D, E, and F are independently selected from carbon or nitrogen.

According to some embodiments, the present disclosure provides a compound according to any of Formulas

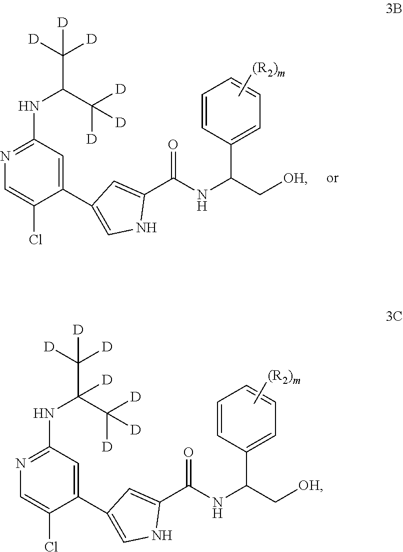

or a pharmaceutically acceptable salt, solvate, or prodrug thereof, wherein R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; and m is 0, 1, or 2.

According to some embodiments, the present disclosure provides a compound according to Formula 4:

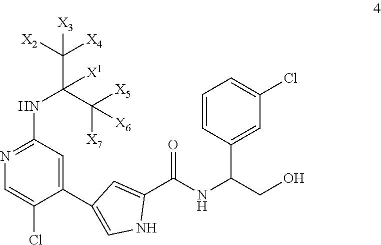

or a pharmaceutically acceptable salt, solvate, or prodrug thereof, wherein: each X₁, X₂, X₃, X₄, X₅, X₆, and X₇ are independently selected from hydrogen and deuterium or C₁₋₄ aliphatic.

In some embodiments, the compound disclosed herein is selected from:
4-(5-chloro-2-((propan-2-yl-d7)amino)pyridin-4-yl)-N-(1-(3-chlorophenyl)-2-hydroxyethyl)-1H-pyrrole-2-carboxamide,
4-(5-chloro-2-((propan-2-yl-1,1,1,3,3,3-d6)amino)pyridin-4-yl)-N-(1-(3-chiorophenyl)-2-hydroxyethyl)-1H-pyrrole-2-carboxamide,
4-(5-chloro-2-((propan-2-yl-1,1,1,2,3-d5)amino)pyridin-4-yl)-N-(1-(3-chlorophenyl)-2-hydroxyethyl)-1H-pyrrole-2-carboxamide,
4-(5-chloro-2-((propan-2-yl-1,1,1,2-d4)amino)pyridin-4-yl)-N-(1-(3-chlorophenyl)-2-hydroxyethyl)-1H-pyrrole-2-carboxamide,
4-(5-chloro-2-((propan-2-yl-1,1,2-d3)amino)pyridin-4-yl)-N-(1-(3-chlorophenyl)-2-hydroxyethyl)-1H-pyrrole-2-carboxamide,
4-(5-chloro-2-((propan-2-yl-1,2-d2)amino)pyridin-4-yl)-N-(1-(3-chlorophenyl)-2-hydroxyethyl)-1H-pyrrole-2-carboxamide,
4-(5-chloro-2-((propan-2-yl-2-d1)amino)pyridin-4-yl)-N-(1-(3-chlorophenyl)-2-hydroxyethyl)-1H-pyrrole-2-carboxamide, and pharmaceutically acceptable salts, solvates, and prodrugs thereof.

In some embodiments, the compound is a substantially pure enantiomer of S- or R-ulixertinib analog. In some embodiments, the compound is a substantially pure enantiomer of 1S-ulixertinib analog. In some embodiments, the compound is a substantially pure enantiomer of 1R-ulixertinib analog. In some embodiments, the compound is an enantiomeric mixture of S- or R-ulixertinib analog. In some embodiments, the compound is a predominantly (i.e., greater than 50%) S-ulixertinib analog. In some embodiments, the compound is a predominantly (i.e., greater than 50%) R-ulixertinib analog. In some embodiments, the compound is an enantiomeric mixture of equal parts S- and R-ulixertinib analog.

In some embodiments, any atom not designated as deuterium is present at its natural isotopic abundance. In some embodiments, each of said positions having deuterium has deuterium enrichment of at least 1%.

According to some aspects the present disclosure provides a pharmaceutical composition comprising a compound disclosed herein and a pharmaceutically acceptable carrier, excipient or vehicle.

According to some aspects, the present disclosure provides a method of treating a disease, disorder, or condition comprising the step of administering a therapeutically effective amount of a compound as disclosed herein to a subject in need thereof, wherein the disease, disorder, or condition comprises one or more of cancer, autoimmune disorders, neurodegenerative and neurological disorders, schizophrenia, bone-related disorders, liver disease, and cardiac disorders. In some embodiments, after administering the therapeutically effective amount of the compound, there is a decreased rate of metabolism of the compound per dosage unit thereof by at least one polymorphically-expressed cytochrome P₄₅₀ isoform as compared to the corresponding non-isotopically enriched compound. In some embodiments, the cytochrome P₄₅₀ isoform is selected from the group consisting of CYP3A4, CYP3A5, CYP2C8, CYP2C9, CYP2D6, CYP2C19, CYP1A2, CYP2B6, and CYP2E1. In some embodiments, the compound has decreased inhibition of at least one cytochrome P₄₅₀ per dosage unit thereof as compared to the non-isotopically enriched compound. In some embodiments, the cytochrome P₄₅₀ is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3ASP1, CYPa5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

According to some aspects, the present disclosure provides a compound or pharmaceutical composition disclosed herein for use as a medicament. In some embodiments, the medicament is for the prevention or treatment of a disorder ameliorated by the inhibition of ERK protein kinase.

According to some aspects, the compound or pharmaceutical composition disclosed herein having deuterium has at least 1% incorporation of deuterium. In some embodiments, at least one of X₁, X₂, X₃, X₄, X₅, X₆, and X₇ is deuterium. In some embodiments, at least two of X₁, X₂, X₃, X₄, X₅, X₆, and X₇ is deuterium. In some embodiments, at least three of X₁, X₂, X₃, X₄, X₅, X₆, and X₇ is deuterium. In some embodiments, at least four of X₁, X₂, X₃, X₄, X₅, X₆, and X₇ is deuterium. In some embodiments, at least five of X₁, X₂, X₃, X₄, X₅, X₆, and X₇ is deuterium. In some embodiments, at least six of X₁, X₂, X₃, X₄, X₅, X₆, and X₇ is deuterium. In some embodiments, each of X₁, X₂, X₃, X₄, X₅, X₆, and X₇ is deuterium. In some embodiments, each of X₂, X₃, X₄, X₅, X₆, and X₇ is deuterium, and X₁ is hydrogen. In some embodiments, each of X₂, X₃, X₄, X₅, X₆, and X₇ is hydrogen, and X₁ is deuterium.

According to some aspects, the present disclosure provides a method of inhibiting ERK1/2 in a cell comprising the step of contacting the cell with the compound or pharmaceutical composition disclosed herein.

In some embodiments, the compound or pharmaceutical composition is effective to decreased metabolism of the compound by at least one polymorphically-expressed cytochrome P₄₅₀ isoform as compared to the corresponding non-isotopically enriched compound. In some embodiments, the cytochrome P₄₅₀ isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3ASP1, CYPa5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51. In some embodiments, the cytochrome P₄₅₀ isoform is selected from the group consisting of CYP3A4, CYP3A5, CYP2C8, CYP2C9, CYP2D6, CYP2C19, CYP1A2, CYP2B6, and CYP2E1. In some embodiments, the decrease in metabolism is greater than about 5%, greater than about 10%, greater than about 20%, greater than about 30%, greater than about 35%, greater than about 40%, greater than about 45%, greater than about 50%, or greater than about 55% as compared to non-isotopically enriched compound. In some embodiments, the compound or pharmaceutical composition is effective to decrease metabolism of the compound in a human cell by at least one polymorphically-expressed cytochrome P₄₅₀ isoform as compared to the corresponding non-isotopically enriched compound by greater than about 10% greater than about 20%, greater than about 30%, greater than about 35%, greater than about 40%, greater than about 50%, greater than about 55%, or greater than about 60%.

According to some aspects, the present disclosure provides a method of synthesizing a deuterated compound according to Formula I:

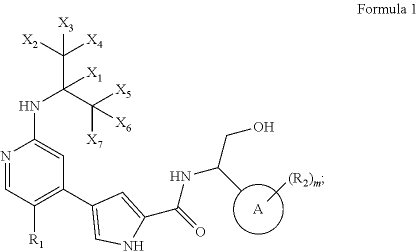

comprising the steps of: (i) reacting a compound of Formula 5:

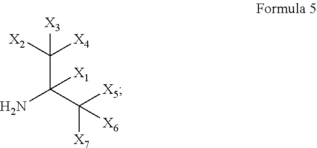

with a compound of Formula 6:

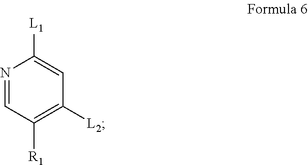

to produce the compound of Formula 7:

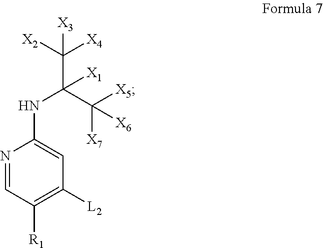

(ii) reacting the compound of Formula 7 with the compound of Formula 8:

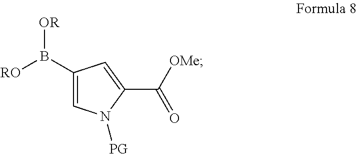

to produce the compound of Formula 9:

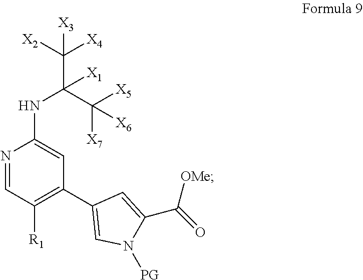

(iii) reacting the compound of Formula 9 with LiOH to produce the compound of Formula 10:

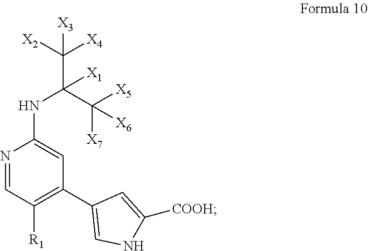

(iv) reacting the compound of Formula 10 with the compound of Formula 11:

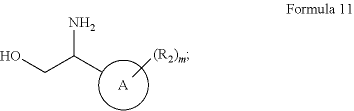

to produce the compounds of Formula 1:

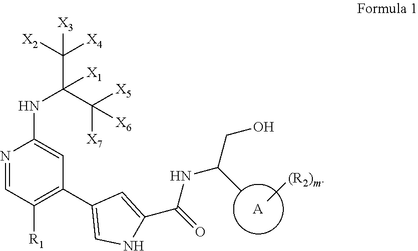

wherein each of X₁, X₂, X₃, X₄, X₅, X₆, and X₇ is independently selected from the group consisting of hydrogen, deuterium, and C₁₋₄ aliphatic, and wherein:
R₁ is hydrogen, C₁₋₃ aliphatic, fluoro, or chloro; A is an optionally substituted group selected from phenyl, a 5-6 membered monocyclic heteroaryl ring having 1-5 heteroatoms independently selected from nitrogen, oxygen, or sulfur, or a 5-6 membered saturated or partially unsaturated heterocyclic ring having 1-5 heteroatoms independently selected from nitrogen, oxygen, or sulfur; R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂; wherein each R is independently hydrogen or C₁₋₄ aliphatic.
m is 0, 1, or 2, PG is a protecting group; and L₁ and L₂ are independently selected leaving groups.

According to some aspects, the present disclosure provides a method of synthesizing a deuterated compound according to Formula 1:

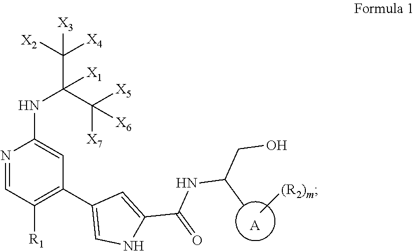

comprising the steps of: (i) reacting a compound of Formula 12:

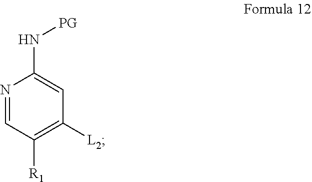

with a compound of Formula 8:

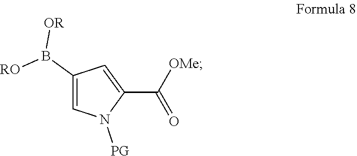

to produce a compound of Formula 13:

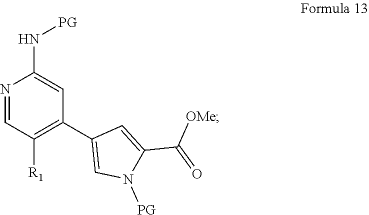

(ii) reacting the compound of Formula 13 with LiOH to produce the compound of Formula 14:

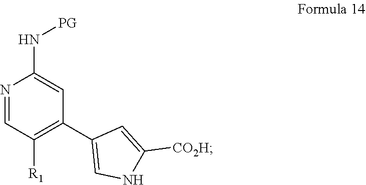

(iii) reacting the compound of Formula 14 with

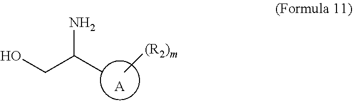

to produce the compound of Formula 15:

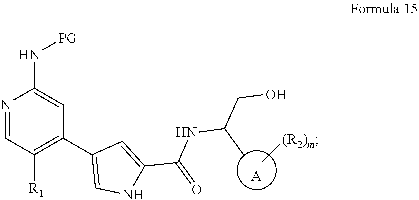

(iv) deprotecting the compound of Formula 15 to produce the compound of Formula 16:

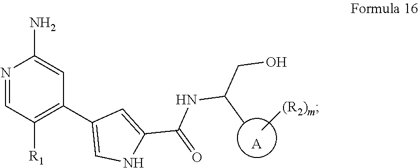

(v) reacting the compound of Formula 16 with the compound of Formula 17:

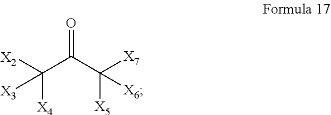

to produce the compounds of Formula 1:

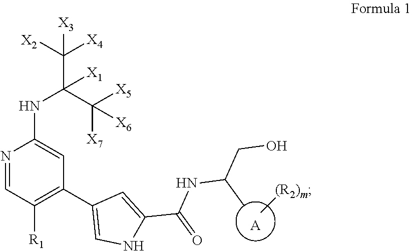

wherein each of X₁, X₂, X₃, X₄, X₅, X₆, and X₇ is independently selected from the group consisting of hydrogen, deuterium, and C₁₋₄ aliphatic, and wherein:
R₁ is hydrogen, C₁₋₃ aliphatic, fluoro, or chloro; A is an optionally substituted group selected from phenyl, a 5-6 membered monocyclic heteroaryl ring having 1-5 heteroatoms independently selected from nitrogen, oxygen, or sulfur, or a 5-6 membered saturated or partially unsaturated heterocyclic ring having 1-5 heteroatoms independently selected from nitrogen, oxygen, or sulfur; R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂; wherein each R is independently hydrogen or C₁₋₄ aliphatic, m is 0, 1, or 2, PG is a protecting group; and L₂ is a leaving group.

According to some embodiments, the above general processes can be used to make any of Formulas 18, 19, or 21 disclosed herein.

According to some aspects, the present disclosure provides a method of synthesizing a deuterated ulixertinib of Formula 18:

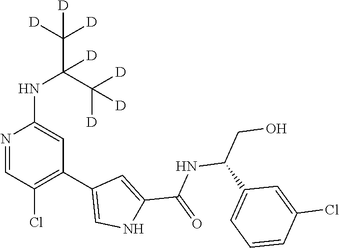 [SMILES: [2H]C([2H])([2H])C([2H])(Nc1cc(-c2c[nH]c(C(=O)N[C@H](CO)c3cccc(Cl)c3)c2)c(Cl)cn1)C([2H])([2H])[2H]]

or a pharmaceutically acceptable salt thereof;
comprising the steps of:
(i) reacting a compound of Formula 7A:

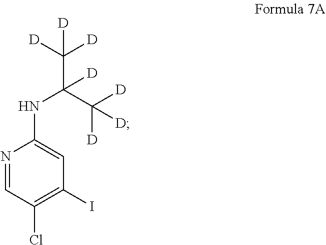

with a compound of Formula 8A:

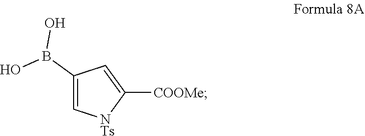 [SMILES: COC(=O)c1cc(B(O)O)cn1S(=O)(=O)c1ccc(C)cc1]

to provide the compound of Formula 9A:

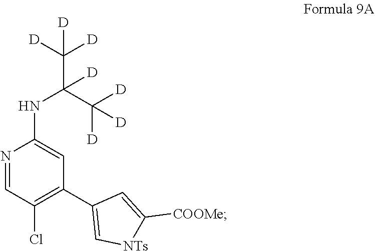 [SMILES: [2H]C([2H])([2H])C([2H])(Nc1cc(-c2cc(C(=O)OC)n(S(=O)(=O)c3ccc(C)cc3)c2)c(Cl)cn1)C([2H])([2H])[2H]]

(ii) reacting the compound of Formula 9A with LiOH to produce the compound of Formula 10A:

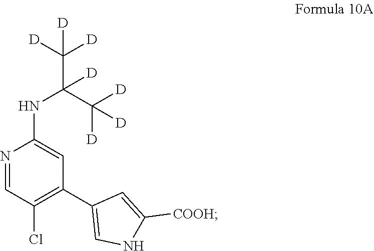 [SMILES: [2H]C([2H])([2H])C([2H])(Nc1cc(-c2c[nH]c(C(=O)O)c2)c(Cl)cn1)C([2H])([2H])[2H]]

and
(iii) reacting the compound of Formula 10A with the compound of Formula 8B:

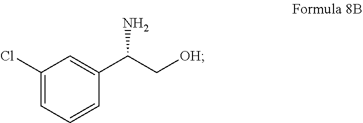 [SMILES: N[C@H](CO)c1cccc(Cl)c1]

to produce the compound of Formula 18:

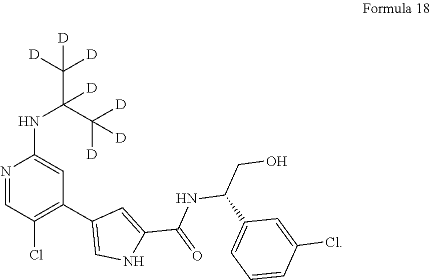 [SMILES: [2H]C([2H])([2H])C([2H])(Nc1cc(-c2c[nH]c(C(=O)N[C@H](CO)c3cccc(Cl)c3)c2)c(Cl)cn1)C([2H])([2H])[2H]]

According to some aspects, the present disclosure provides a method of synthesizing a HCl salt form of deuterated ulixertinib analog comprising the step of reacting a compound of Formula 18:

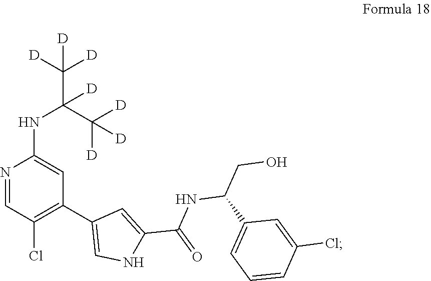 [SMILES: [2H]C([2H])([2H])C([2H])(Nc1cc(-c2c[nH]c(C(=O)N[C@H](CO)c3cccc(Cl)c3)c2)c(Cl)cn1)C([2H])([2H])[2H]]

with HCl to produce the compound of Formula 18A:

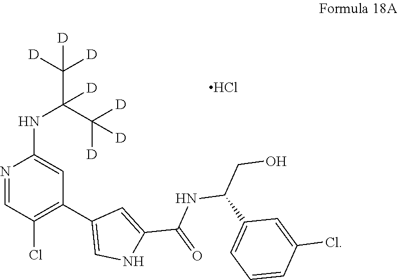 [SMILES: [2H]C([2H])([2H])C([2H])(Nc1cc(-c2c[nH]c(C(=O)N[C@H](CO)c3cccc(Cl)c3)c2)c(Cl)cn1)C([2H])([2H])[2H] ; Cl]

According to some aspects, the present disclosure provides a method of synthesizing a deuterated ulixertinib of Formula 19, or a pharmaceutically acceptable salt thereof:

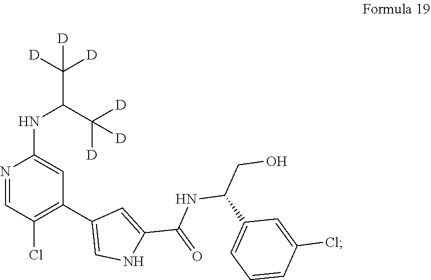 [SMILES: [2H]C([2H])([2H])C(Nc1cc(-c2c[nH]c(C(=O)N[C@H](CO)c3cccc(Cl)c3)c2)c(Cl)cn1)C([2H])([2H])[2H]]

comprising the steps of:
(i) reacting a compound of Formula 12A:

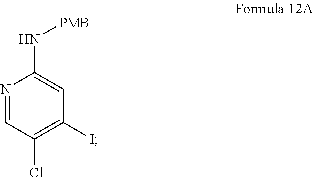

with a compound of Formula 8A:

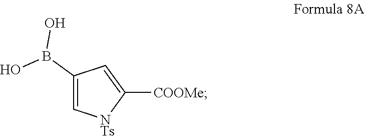 [SMILES: COC(=O)c1cc(B(O)O)cn1S(=O)(=O)c1ccc(C)cc1]

to produce a compound of Formula 13A:

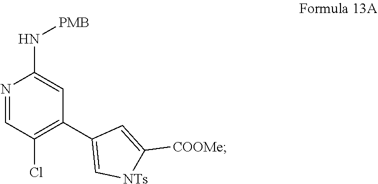

(ii) reacting the compound of Formula 13A with LiOH to produce the compound of Formula 14A:

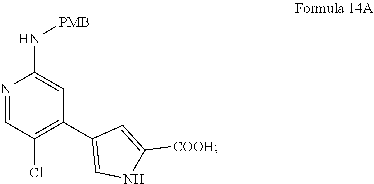

(iii) reacting the compound of Formula 14A with the compound of Formula 8B:

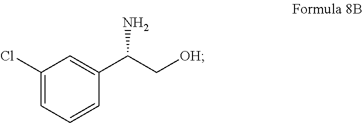 [SMILES: N[C@H](CO)c1cccc(Cl)c1]

to produce a compound of Formula 15A:

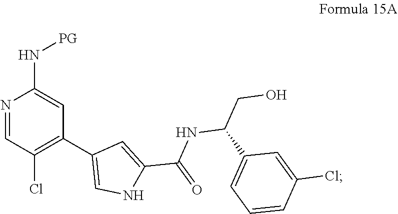

(iv) reacting the compound of Formula 15A with TFA to produce the compound of Formula 16A:

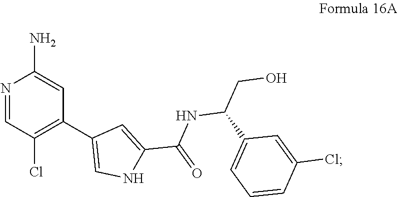 [SMILES: Nc1cc(-c2c[nH]c(C(=O)N[C@H](CO)c3cccc(Cl)c3)c2)c(Cl)cn1]

and (v) reacting the compound of Formula 16A with

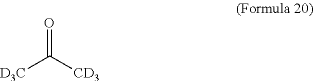

to produce the compound of Formula 19:

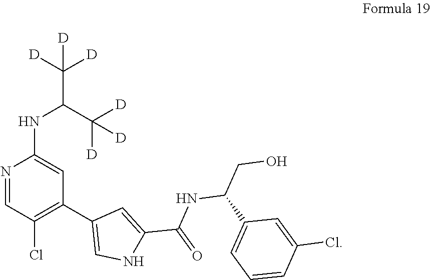 [SMILES: [2H]C([2H])([2H])C(Nc1cc(-c2c[nH]c(C(=O)N[C@H](CO)c3cccc(Cl)c3)c2)c(Cl)cn1)C([2H])([2H])[2H]]

According to some aspects, the present disclosure provides a method of synthesizing a HCl salt form of deuterated ulixertinib analog comprising the steps of reacting a compound of Formula 19:

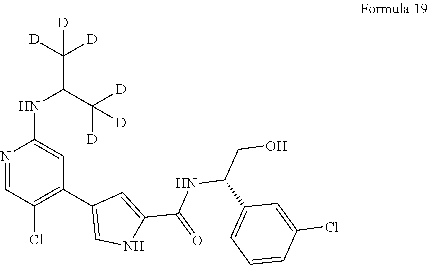 [SMILES: [2H]C([2H])([2H])C(Nc1cc(-c2c[nH]c(C(=O)N[C@H](CO)c3cccc(Cl)c3)c2)c(Cl)cn1)C([2H])([2H])[2H]]

with HCl to produce the compound of Formula 19A:

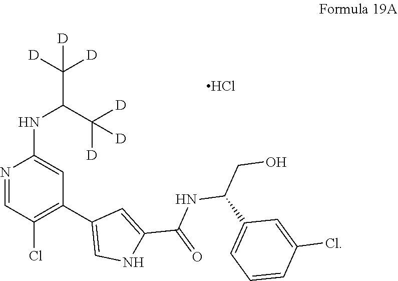 [SMILES: [2H]C([2H])([2H])C(Nc1cc(-c2c[nH]c(C(=O)N[C@H](CO)c3cccc(Cl)c3)c2)c(Cl)cn1)C([2H])([2H])[2H] ; Cl]

According to some aspects, the present disclosure provides a method of synthesizing a deuterated ulixertinib of Formula 21, or a pharmaceutically acceptable salt thereof:

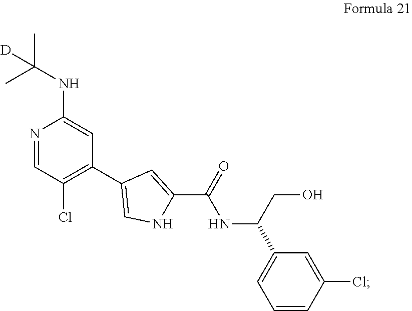 [SMILES: [2H]C(C)(C)Nc1cc(-c2c[nH]c(C(=O)N[C@H](CO)c3cccc(Cl)c3)c2)c(Cl)cn1]

comprising the steps of:
(i) reacting a compound of Formula 7B:

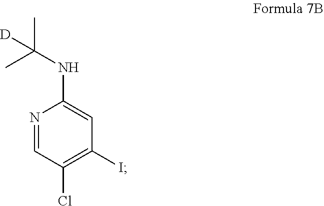

with a compound of Formula 8A:

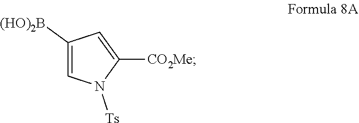 [SMILES: COC(=O)c1cc(B(O)O)cn1S(=O)(=O)c1ccc(C)cc1]

to produce a compound of Formula 9B:

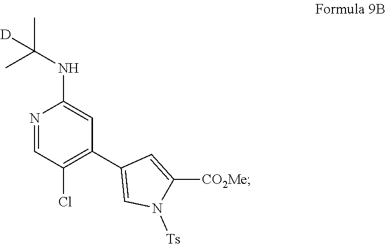 [SMILES: [2H]C(C)(C)Nc1cc(-c2cc(C(=O)OC)n(S(=O)(=O)c3ccc(C)cc3)c2)c(Cl)cn1]

(ii) reacting the compound of Formula 9B with LiOH to produce a compound of Formula 10B:

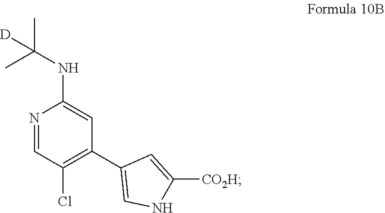 [SMILES: [2H]C(C)(C)Nc1cc(-c2c[nH]c(C(=O)O)c2)c(Cl)cn1]

and (iii) reacting the compound of Formula 10B with the compound of Formula 8B:

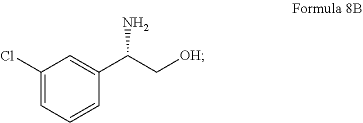 [SMILES: N[C@H](CO)c1cccc(Cl)c1]

to produce the compound of Formula 21:

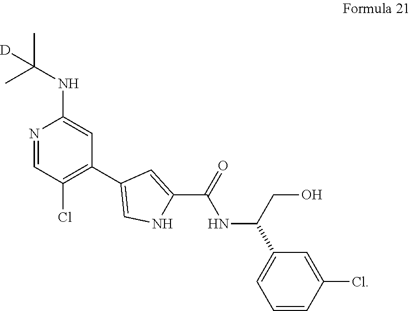 [SMILES: [2H]C(C)(C)Nc1cc(-c2c[nH]c(C(=O)N[C@H](CO)c3cccc(Cl)c3)c2)c(Cl)cn1]

According to some aspects, the present disclosure provides a method of synthesizing a deuterated ulixertinib analog comprising the step of reacting a compound of Formula 21:

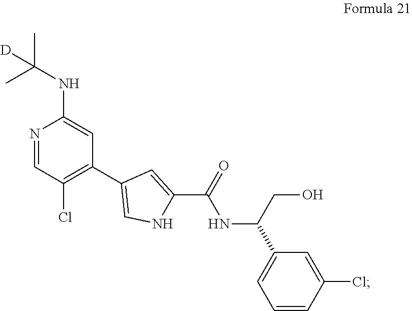 [SMILES: [2H]C(C)(C)Nc1cc(-c2c[nH]c(C(=O)N[C@H](CO)c3cccc(Cl)c3)c2)c(Cl)cn1]

with HCl to produce the compound of Formula 21A:

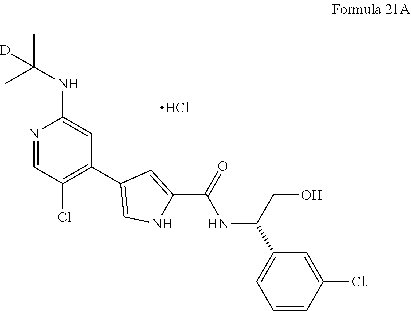 [SMILES: [2H]C(C)(C)Nc1cc(-c2c[nH]c(C(=O)N[C@H](CO)c3cccc(Cl)c3)c2)c(Cl)cn1 ; Cl]

According to some aspects, the present disclosure provides a compound selected from:

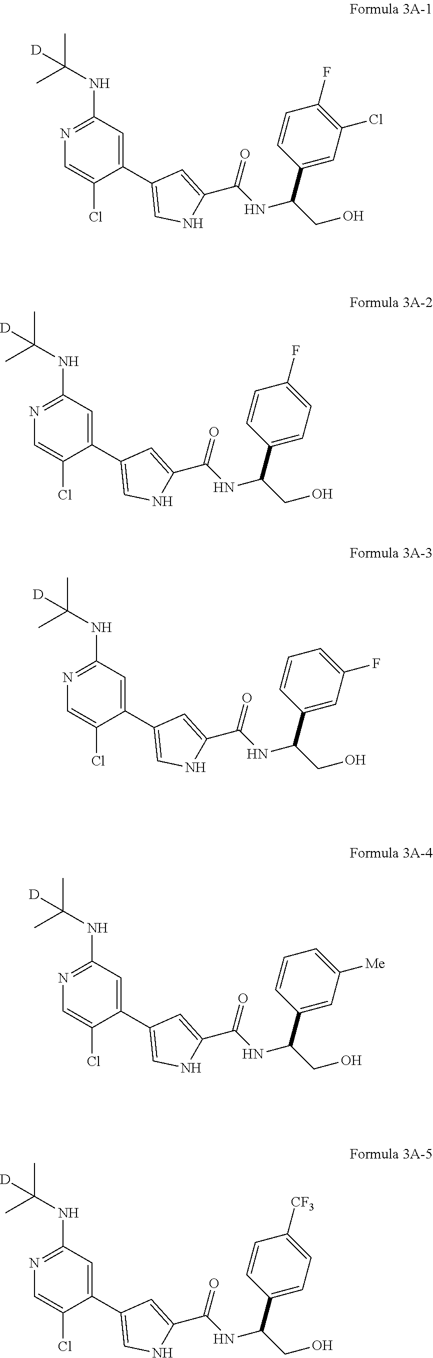 [SMILES: [2H]C(C)(C)Nc1cc(-c2c[nH]c(C(=O)N[C@H](CO)c3ccc(F)c(Cl)c3)c2)c(Cl)cn1 ; [2H]C(C)(C)Nc1cc(-c2c[nH]c(C(=O)N[C@H](CO)c3ccc(F)cc3)c2)c(Cl)cn1 ; [2H]C(C)(C)Nc1cc(-c2c[nH]c(C(=O)N[C@H](CO)c3cccc(F)c3)c2)c(Cl)cn1 ; [2H]C(C)(C)Nc1cc(-c2c[nH]c(C(=O)N[C@H](CO)c3cccc(C)c3)c2)c(Cl)cn1 ; [2H]C(C)(C)Nc1cc(-c2c[nH]c(C(=O)N[C@H](CO)c3ccc(C(F)(F)F)cc3)c2)c(Cl)cn1]

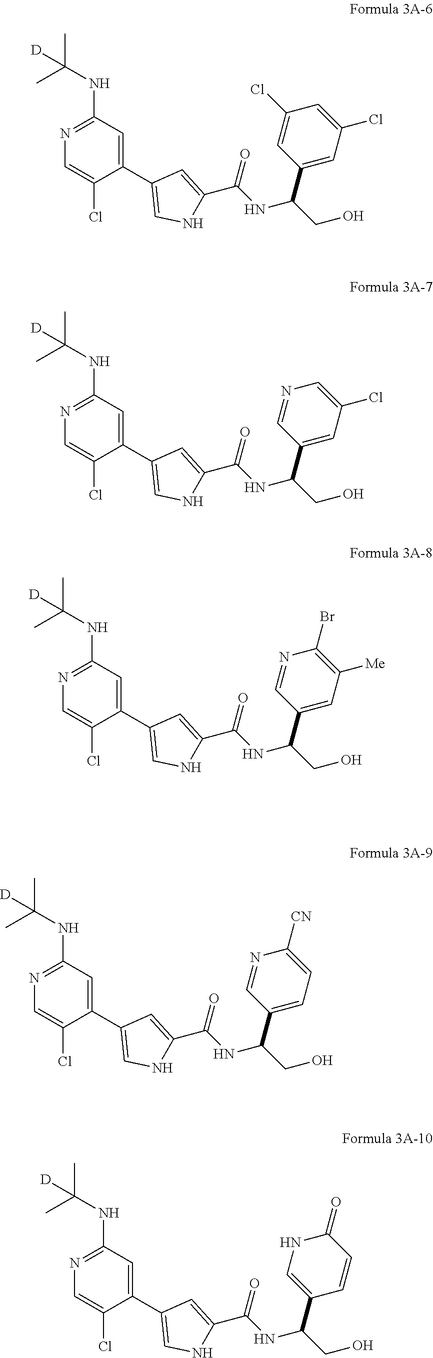 [SMILES: [2H]C(C)(C)Nc1cc(-c2c[nH]c(C(=O)N[C@H](CO)c3cc(Cl)cc(Cl)c3)c2)c(Cl)cn1 ; [2H]C(C)(C)Nc1cc(-c2c[nH]c(C(=O)N[C@H](CO)c3cncc(Cl)c3)c2)c(Cl)cn1 ; [2H]C(C)(C)Nc1cc(-c2c[nH]c(C(=O)N[C@H](CO)c3cnc(Br)c(C)c3)c2)c(Cl)cn1 ; [2H]C(C)(C)Nc1cc(-c2c[nH]c(C(=O)N[C@H](CO)c3ccc(C#N)nc3)c2)c(Cl)cn1 ; [2H]C(C)(C)Nc1cc(-c2c[nH]c(C(=O)N[C@H](CO)c3ccc(=O)[nH]c3)c2)c(Cl)cn1]

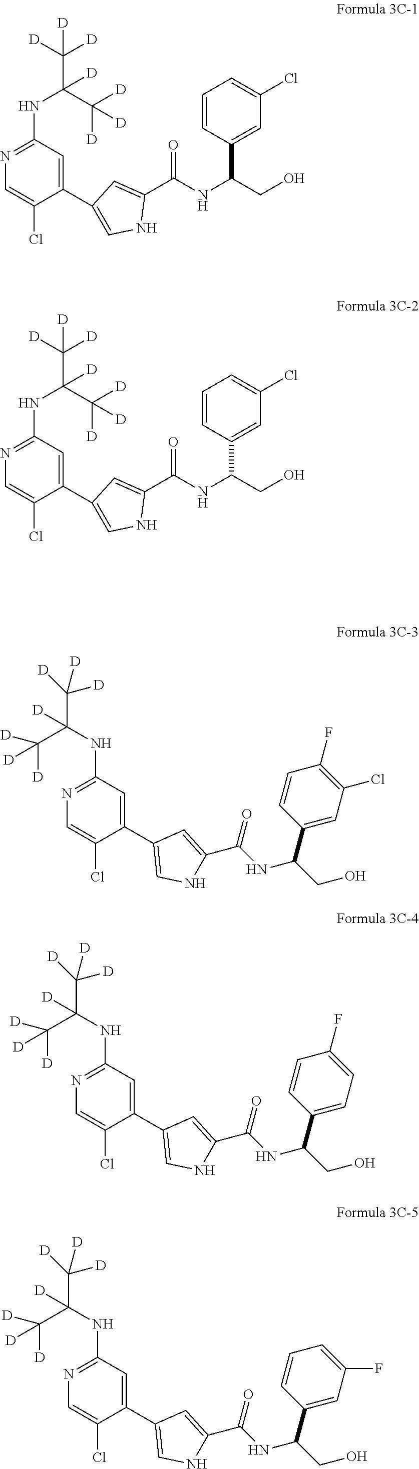

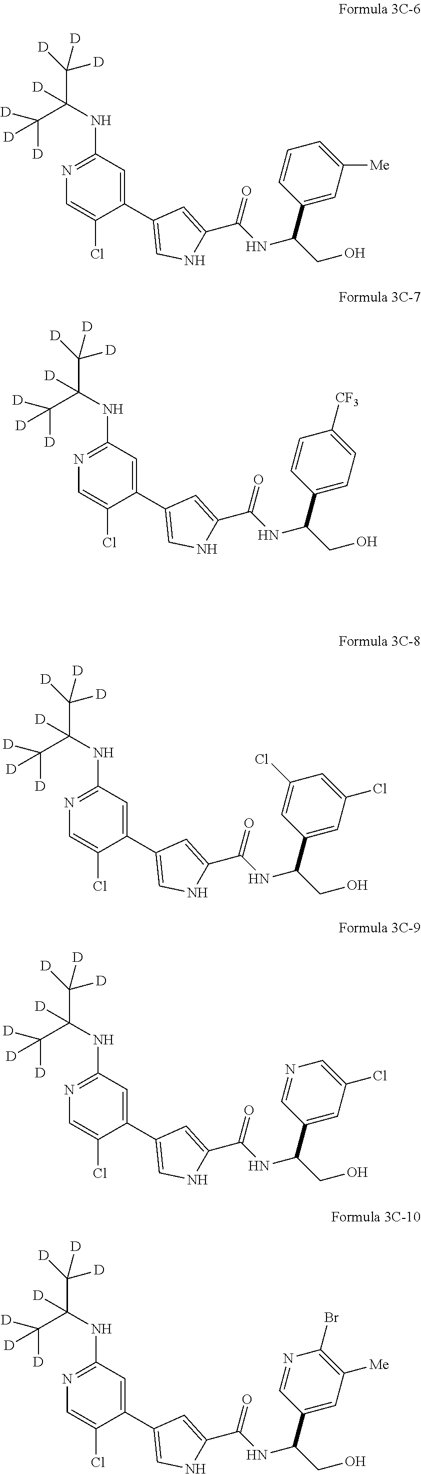

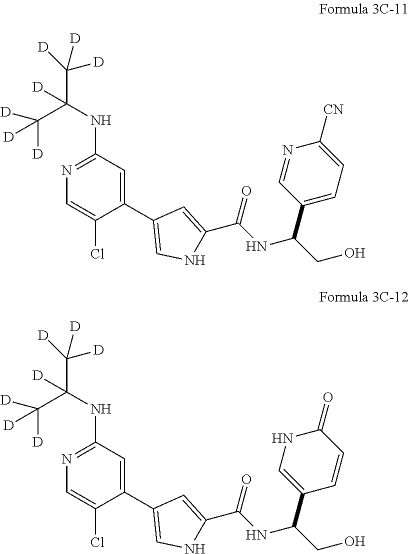 [SMILES: [2H]C([2H])([2H])C([2H])(Nc1cc(-c2c[nH]c(C(=O)N[C@H](CO)c3ccc(C#N)nc3)c2)c(Cl)cn1)C([2H])([2H])[2H] ; [2H]C([2H])([2H])C([2H])(Nc1cc(-c2c[nH]c(C(=O)N[C@H](CO)c3ccc(=O)[nH]c3)c2)c(Cl)cn1)C([2H])([2H])[2H]]

and pharmaceutically acceptable salts, solvates, and prodrugs thereof.

According to some aspects, the present disclosure provides a compound selected from:

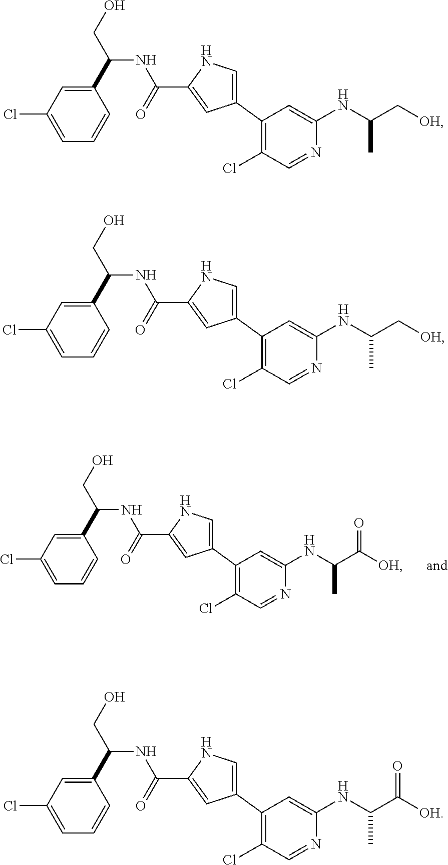

In some aspects, the present disclosure provides a kit for treating or ameliorating the effects of a disease in a subject, the kit comprising a compound or a pharmaceutical composition as disclosed herein packaged together with instructions for its use. In some embodiments, the compound or pharmaceutical composition in the kit is effective to decrease metabolism of the compound or pharmaceutical composition by at least one polymorphically-expressed cytochrome P₄₅₀ isoform as compared to the corresponding non-isotopically enriched compound.

### DETAILED DESCRIPTION

According to some aspects, the present disclosure provides deuterated analogs of ulixertinib of Formula I, solvates, prodrugs, and pharmaceutically acceptable salts thereof, as well as methods for their preparation and use, and to pharmaceutical compositions thereof.

In some embodiments, the deuterated analogs of ulixertinib as disclosed herein are compounds represented by the general structure found in Formula 1:

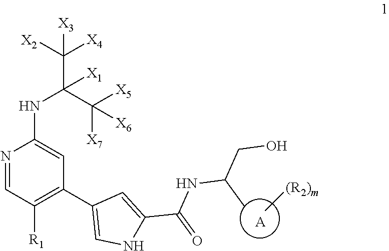

including pharmaceutically acceptable salts, solvates, and prodrugs thereof, wherein each X₁, X₂, X₃, X₄, X₅, X₆, and X₇ are independently selected from the group consisting of hydrogen and deuterium or C₁₋₄ aliphatic, and wherein each of Ring A, R₁, R₂, and m are as defined herein. In some embodiments:
   R₁ is hydrogen, C₁₋₃ aliphatic, fluoro, or chloro;
   Ring A is an optionally substituted group selected from phenyl, 5-6 membered monocyclic heteroaryl ring having 1-5 heteroatoms independently selected from nitrogen, oxygen, or sulfur, or a 5-6 membered saturated or partially unsaturated heterocyclic ring having 1-5 heteroatoms independently selected from nitrogen, oxygen, or sulfur;
   R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂; and
   m is 0, 1, or 2.

According to some embodiments, compounds include those described generally above, and are further illustrated by the classes, subclasses, and species disclosed herein.

### Definitions

The following definitions shall apply unless otherwise indicated. As used herein, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As used herein, the term “prodrug” refers to a derivative of a parent drug molecule that requires transformation within the body in order to release the active drug, and that has improved physical and/or delivery properties over the parent drug molecule. Prodrugs are designed to enhance pharmaceutically and/or pharmacokinetically based properties associated with the parent drug molecule. The advantage of a prodrug lies in its physical properties, such as enhanced water solubility for parenteral administration at physiological pH compared to the parent drug, or it enhances absorption from the digestive tract, or it may enhance drug stability for long-term storage. Using esters as a prodrug type for drugs containing carboxyl or hydroxyl function is known in the art as described, for example, in “The Organic Chemistry of Drug Design and Drug Interaction” Richard Silverman, published by Academic Press (1992).

As used herein “solvate” refers to a complex of variable stoichiometry formed by a solute (e.g. a compound of Formula 1 or a salt or prodrug thereof) and a solvent. Such solvents for the purpose of this disclosure may not interfere with the biological activity of the solute. Examples of suitable solvents include water, methanol, ethanol and acetic acid. Generally the solvent used is a pharmaceutically acceptable solvent. Examples of suitable pharmaceutically acceptable solvents include water, ethanol and acetic acid. Generally the solvent used is water.

As described herein, compounds disclosed herein may optionally be substituted with one or more substituents, such as are illustrated generally above, or as exemplified by particular classes, subclasses, and species as disclosed herein. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position.

Combinations of substituents envisioned by this disclosure are preferably those that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. In certain embodiments, aliphatic groups contain 1-6 aliphatic carbon atoms, and in yet other embodiments, aliphatic groups contain 1-4 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “unsaturated”, as used herein, means that a moiety has one or more units of unsaturation.

The terms “haloalkyl”, “haloalkenyl” and “haloalkoxy” means alkyl, alkenyl or alkoxy, as the case may be, substituted with one or more halogen atoms. The term “halogen” means F, Cl, Br, or T.

The term “aryl” used alone or as part of a larger moiety as in “aralkyl”, “aralkoxy”, or “aryloxyalkyl”, refers to monocyclic, bicyclic and tricyclic ring systems having a total of five to fourteen ring members, wherein at least one ring in the system is aromatic and wherein each ring in the system contains 3 to 7 ring members. The term “aryl” may be used interchangeably with the term “aryl ring”.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of this disclosure. Unless otherwise stated, all tautomeric forms of the compounds are within the scope of this disclosure. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure.

It will be recognized that some variation of natural isotopic abundance occurs in a synthesized compound depending upon the origin of chemical materials used in the synthesis. Thus, a preparation of the compounds disclosed herein will inherently contain small amounts of deuterated isotopologues. The term “isotopologue” refers to a species that differs from a specific compound disclosed herein only in the isotopic composition thereof. The concentration of naturally abundant stable hydrogen and carbon isotopes, notwithstanding this variation, is small and immaterial as compared to the degree of stable isotopic substitution of compounds of this disclosure. See, for instance. Wada, E et al., Seikagaku, 1994, 66:15; Gannes, L Z et al., Comp Biochem Physiol Mol Integr Physiol, 1998, 119:725.

In the compounds disclosed herein, any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also, unless otherwise stated, when a position is designated specifically as “D” or “deuterium”, the position is understood to have deuterium at an abundance that is at least 66 times greater than the natural abundance of deuterium, which is 0.015% (i.e., at least 10% incorporation of deuterium).

The term “isotopic enrichment factor” as used herein means the ratio between the isotopic abundance and the natural abundance of a specified isotope. In some embodiments, a compound disclosed herein has an isotopic enrichment factor for each designated deuterium atom of at least 66 (1% incorporation at each designated deuterium atom), at least 1666 (25% deuterium incorporation), at least 3333 (50% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6533 (98% deuterium incorporation), at least 6600 (99% deuterium incorporation), or at least 6633.3 (99.5% deuterium incorporation).

The term “compound,” when referring to a compound disclosed herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound disclosed herein will depend upon a number of factors including the isotopic purity of deuterated reagents used to make the compound and the efficiency of incorporation of deuterium in the various synthesis steps used to prepare the compound. However, as set forth above, the relative amount of such isotopologues in total will be less than 49.9% of the compound. In other embodiments, the relative amount of such isotopologues in total will be less than 47.5%, less than 40%, less than 32.5%, less than 25%, less than 17.5%, less than 10%, less than 5%, less than 3%, less than 1%, or less than 0.5/0 of the compound.

The compounds disclosed herein (including, compounds of Formula 1), may contain an asymmetric carbon atom, for example, as the result of deuterium substitution or otherwise. As such, compounds disclosed herein can exist as either individual enantiomers, or mixtures of enantiomers. Accordingly, a compound disclosed herein may exist as either a racemic mixture or a scalemic mixture, or as individual respective stereoisomers that are substantially free from another possible stereoisomer. The term “substantially free of other stereoisomers” or “substantially pure enantiomer” as used herein means less than 25% of other stereoisomers, less than 10% of other stereoisomers, less than 5% of other stereoisomers, and less than 2% of other stereoisomers, or less than “X”% of other stereoisomers (wherein X is a number between 0 and 100, inclusive) are present. Methods of obtaining or synthesizing an individual enantiomer for a given compound are known in the art and may be applied as practicable to final compounds or to starting material or intermediates.

The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other.

It is appreciated that compounds of the present invention having a chiral center may exist in and be isolated in optically active and racemic forms. Some compounds may exhibit polymorphism. It is to be understood that the present invention encompasses any racemic, optically-active, diastereomeric, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Methods for obtaining enriched or pure enantiomers include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts as disclosed in more detail herein or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The stereoisomers may also be separated by usual techniques known to those skilled in the art including fractional crystallization of the bases or their salts or chromatographic techniques such as LC or flash chromatography. The (+) enantiomer can be separated from the (−) enantiomer using techniques and procedures well known in the art, such as that described by J. Jacques, et al., antiomers, Racemates, and Resolutions”, John Wiley and Sons, Inc., 1981. For example, chiral chromatography with a suitable organic solvent, such as ethanol/acetonitrile and Chiralpak AD packing, 20 micron can also be utilized to effect separation of the enantiomers.

“D” and “d” both refer to deuterium.

The term “optionally substituted with deuterium” means that one or more hydrogen atoms in the referenced moiety may be replaced with a corresponding number of deuterium atoms.

The present disclosure provides prodrugs of the compounds of Formula 1 above. In general, such prodrugs will be functional derivatives of the compounds of Formula 1 that are readily convertible in vivo into the required compound of Formula 1. Conventional procedures for the selection and preparation of suitable prodrug derivatives are described, for example, in Design of Prodrugs, ed. H. Bundgaard. Elsevier, 1985. Such prodrugs include but are not limited to ester prodrugs from alcohols and acids and phosphate prodrugs of alcohols. The prodrug can be formulation to achieve a goal of improved chemical stability, improved patient acceptance and compliance, improved bioavailability, prolonged duration of action, improved organ selectivity, improved formulation (including, increased hydrosolubility), and/or decreased side effects (including, toxicity).

The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of the deuterated compounds disclosed herein.

### Therapeutic Compounds

In some embodiments, the present disclosure provides a deuterated pyridinylpyrrole carboxamide analog according to Formula 1:

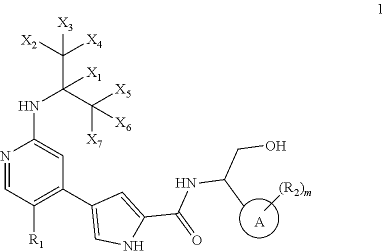

where Ring A, the number and type of substituents, and the substitution patterns on the ring are varied.

In one aspect, the present disclosure provides compounds of Formulae 2B-1 and 2B-2:

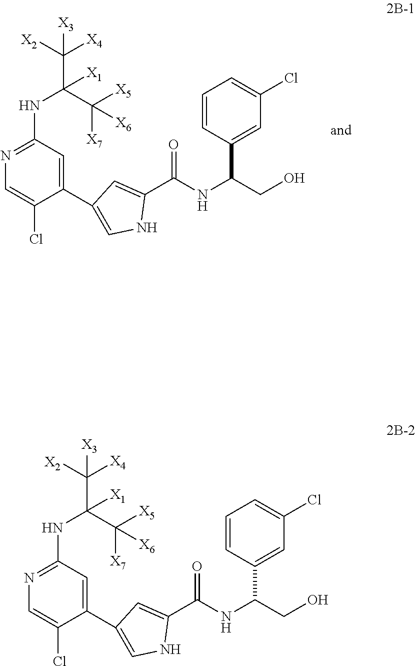

or pharmaceutically acceptable salts, solvates, and prodrugs thereof, wherein:
   each of X₁, X₂, X₃, X₄, X₅, X₆, and X₇ are independently selected from hydrogen and deuterium.

In some embodiments, X₁, X₂, X₃, X₄, X₅, X₆, and X₇ are deuterium such that the deuterated analog of ulixertinib is a compound having the structure of Formulae 3C-1 and 3C-2:

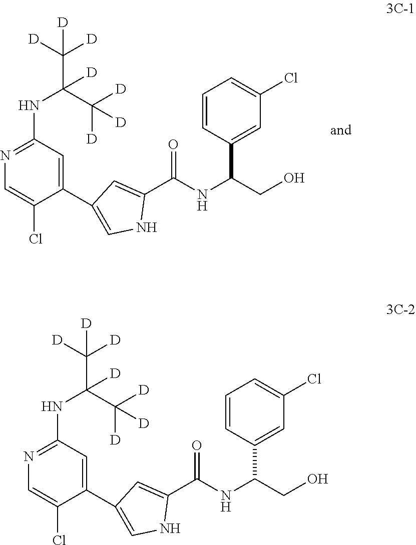 [SMILES: [2H]C([2H])([2H])C([2H])(Nc1cc(-c2c[nH]c(C(=O)N[C@H](CO)c3cccc(Cl)c3)c2)c(Cl)cn1)C([2H])([2H])[2H] ; [2H]C([2H])([2H])C([2H])(Nc1cc(-c2c[nH]c(C(=O)N[C@@H](CO)c3cccc(Cl)c3)c2)c(Cl)cn1)C([2H])([2H])[2H]]

and pharmaceutically acceptable salts, solvates, and prodrugs thereof.

In some embodiments, the compounds of Formulas 2B-1 and 2B-2 have X₂, X₃, X₄, X₅, X₆, and X₇ as hydrogen such that the deuterated analog of ulixertinib is a compound having the structure of Formulae 3A-11 and 3A-12:

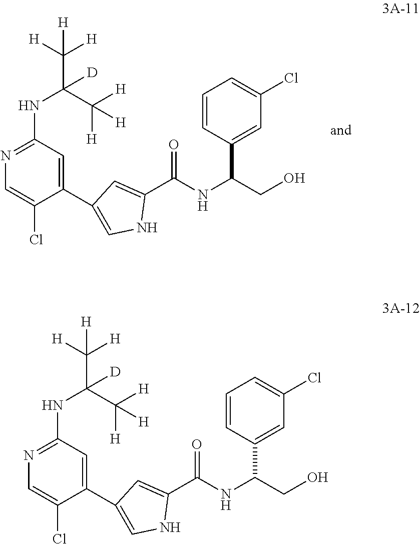

In some embodiments, the compounds of Formulas 2B-1 and 2B-2 have X₁ as hydrogen, and X₂, X₃, X₄, X₅, X₆, and X₇ are deuterium such that the deuterated analog of ulixertinib is a compound having the structure of Formulae 3B-1 and 3B-2:

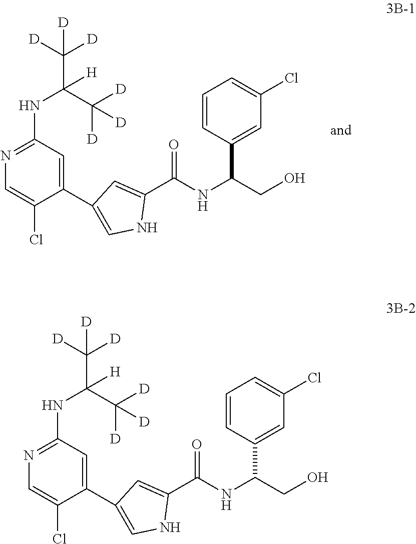

In yet other embodiments, there are provided compounds according to Formulae 2B-1 and 2B-2 having one of the following structures:

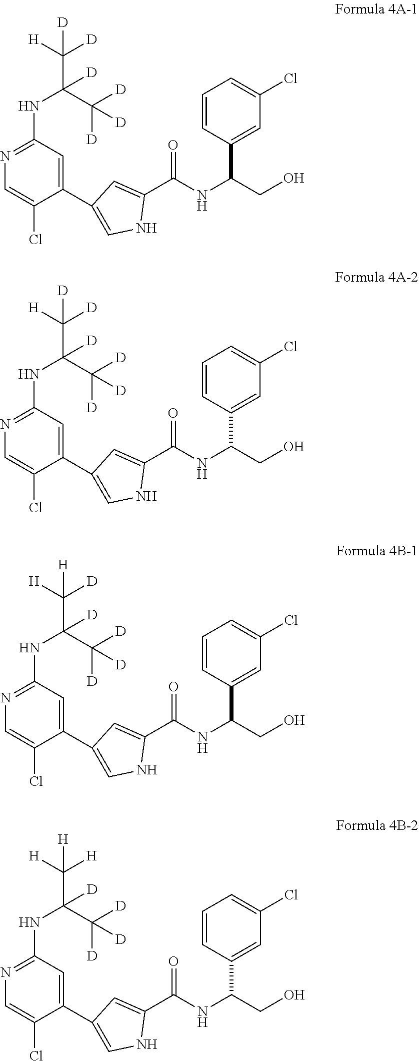 [SMILES: [2H]C([2H])C([2H])(Nc1cc(-c2c[nH]c(C(=O)N[C@H](CO)c3cccc(Cl)c3)c2)c(Cl)cn1)C([2H])([2H])[2H] ; [2H]C([2H])C([2H])(Nc1cc(-c2c[nH]c(C(=O)N[C@@H](CO)c3cccc(Cl)c3)c2)c(Cl)cn1)C([2H])([2H])[2H] ; [2H]CC([2H])(Nc1cc(-c2c[nH]c(C(=O)N[C@H](CO)c3cccc(Cl)c3)c2)c(Cl)cn1)C([2H])([2H])[2H] ; [2H]C([2H])([2H])C([2H])(C)Nc1cc(-c2c[nH]c(C(=O)N[C@@H](CO)c3cccc(Cl)c3)c2)c(Cl)cn1]

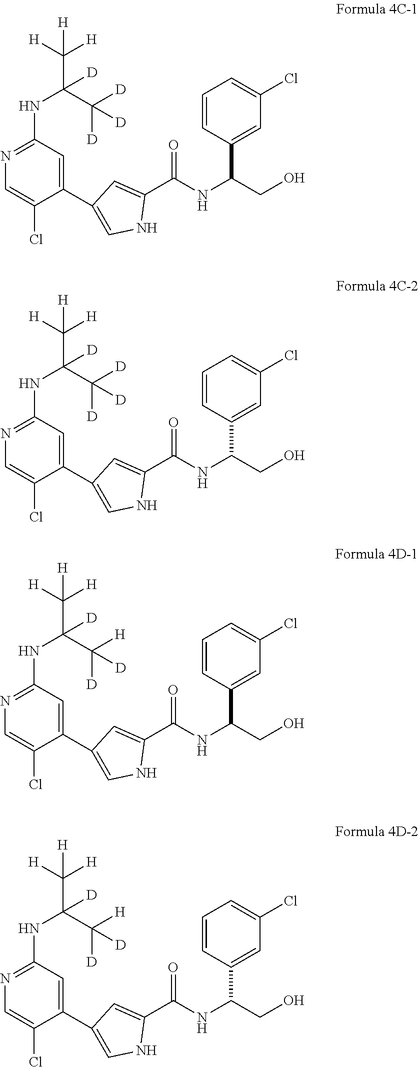 [SMILES: [2H]C([2H])([2H])C([2H])(C)Nc1cc(-c2c[nH]c(C(=O)N[C@H](CO)c3cccc(Cl)c3)c2)c(Cl)cn1 ; [2H]C([2H])([2H])C([2H])(C)Nc1cc(-c2c[nH]c(C(=O)N[C@@H](CO)c3cccc(Cl)c3)c2)c(Cl)cn1 ; [2H]C([2H])C([2H])(C)Nc1cc(-c2c[nH]c(C(=O)N[C@H](CO)c3cccc(Cl)c3)c2)c(Cl)cn1 ; [2H]C([2H])C([2H])(C)Nc1cc(-c2c[nH]c(C(=O)N[C@@H](CO)c3cccc(Cl)c3)c2)c(Cl)cn1]

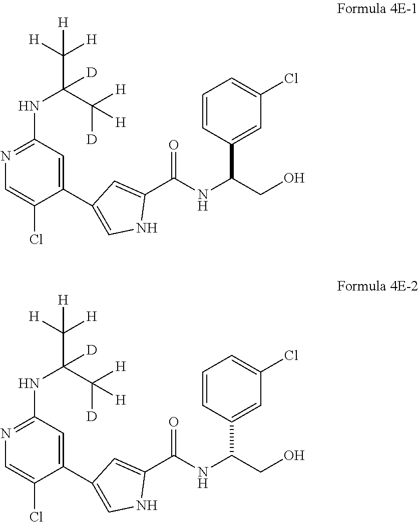 [SMILES: [2H]CC([2H])(C)Nc1cc(-c2c[nH]c(C(=O)N[C@H](CO)c3cccc(Cl)c3)c2)c(Cl)cn1 ; [2H]CC([2H])(C)Nc1cc(-c2c[nH]c(C(=O)N[C@@H](CO)c3cccc(Cl)c3)c2)c(Cl)cn1]

Additional non-limiting exemplary embodiments of the compound of Formula 1 include:

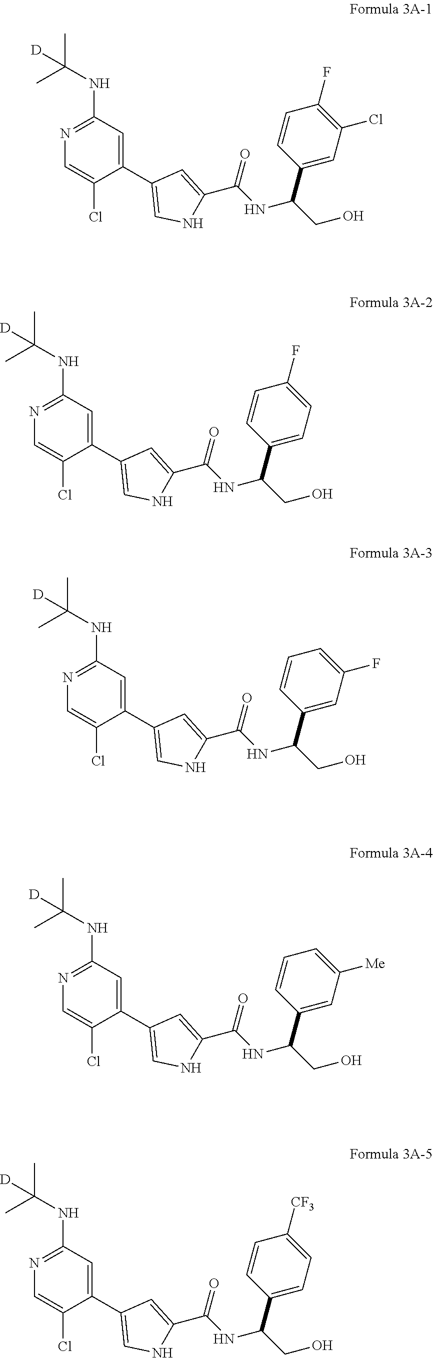 [SMILES: [2H]C(C)(C)Nc1cc(-c2c[nH]c(C(=O)N[C@H](CO)c3ccc(F)c(Cl)c3)c2)c(Cl)cn1 ; [2H]C(C)(C)Nc1cc(-c2c[nH]c(C(=O)N[C@H](CO)c3ccc(F)cc3)c2)c(Cl)cn1 ; [2H]C(C)(C)Nc1cc(-c2c[nH]c(C(=O)N[C@H](CO)c3cccc(F)c3)c2)c(Cl)cn1 ; [2H]C(C)(C)Nc1cc(-c2c[nH]c(C(=O)N[C@H](CO)c3cccc(C)c3)c2)c(Cl)cn1 ; [2H]C(C)(C)Nc1cc(-c2c[nH]c(C(=O)N[C@H](CO)c3ccc(C(F)(F)F)cc3)c2)c(Cl)cn1]

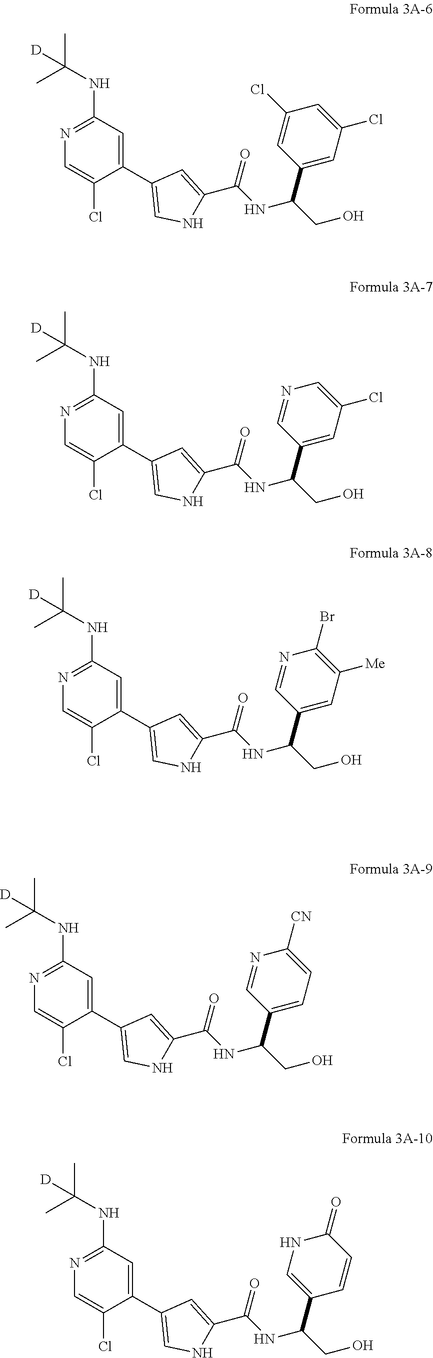 [SMILES: [2H]C(C)(C)Nc1cc(-c2c[nH]c(C(=O)N[C@H](CO)c3cc(Cl)cc(Cl)c3)c2)c(Cl)cn1 ; [2H]C(C)(C)Nc1cc(-c2c[nH]c(C(=O)N[C@H](CO)c3cncc(Cl)c3)c2)c(Cl)cn1 ; [2H]C(C)(C)Nc1cc(-c2c[nH]c(C(=O)N[C@H](CO)c3cnc(Br)c(C)c3)c2)c(Cl)cn1 ; [2H]C(C)(C)Nc1cc(-c2c[nH]c(C(=O)N[C@H](CO)c3ccc(C#N)nc3)c2)c(Cl)cn1 ; [2H]C(C)(C)Nc1cc(-c2c[nH]c(C(=O)N[C@H](CO)c3ccc(=O)[nH]c3)c2)c(Cl)cn1]

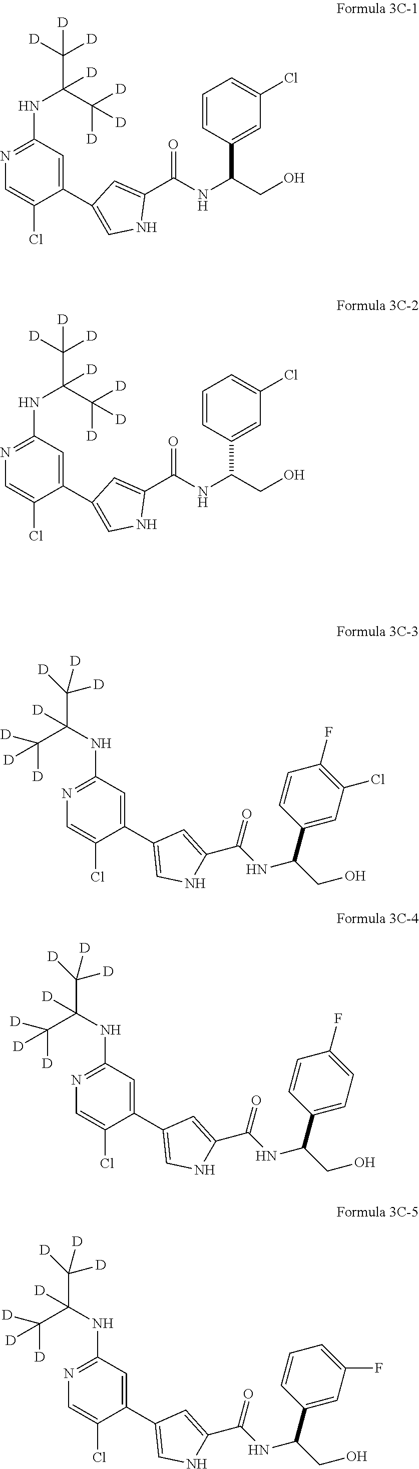

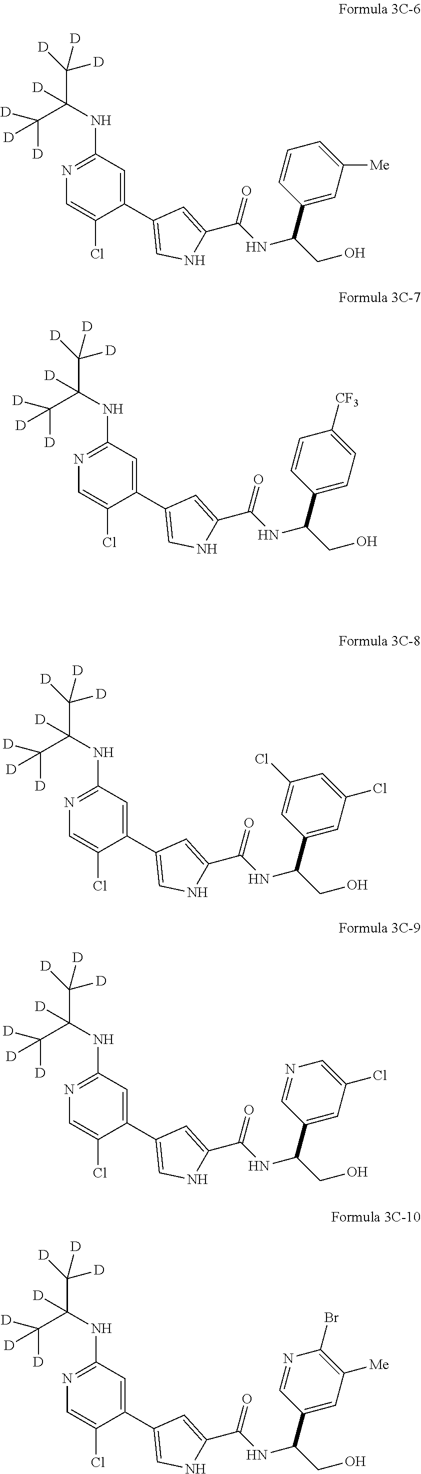

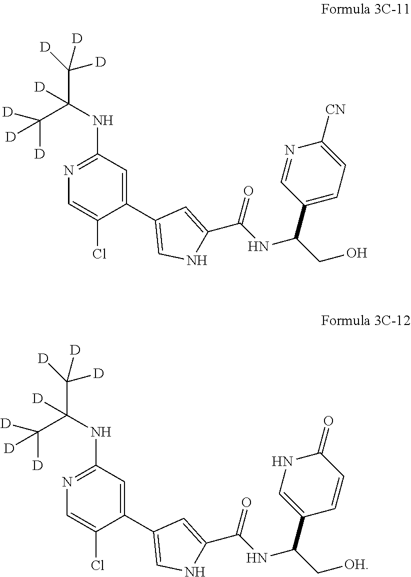 [SMILES: [2H]C([2H])([2H])C([2H])(Nc1cc(-c2c[nH]c(C(=O)N[C@H](CO)c3ccc(C#N)nc3)c2)c(Cl)cn1)C([2H])([2H])[2H] ; [2H]C([2H])([2H])C([2H])(Nc1cc(-c2c[nH]c(C(=O)N[C@H](CO)c3ccc(=O)[nH]c3)c2)c(Cl)cn1)C([2H])([2H])[2H]]

### General Methods of Preparing Compounds

In some embodiments, the compounds disclosed herein may be prepared or isolated in general by synthetic methods known to those skilled in the art for analogous compounds and as illustrated by the general schemes I-V below and the preparative examples that follow.

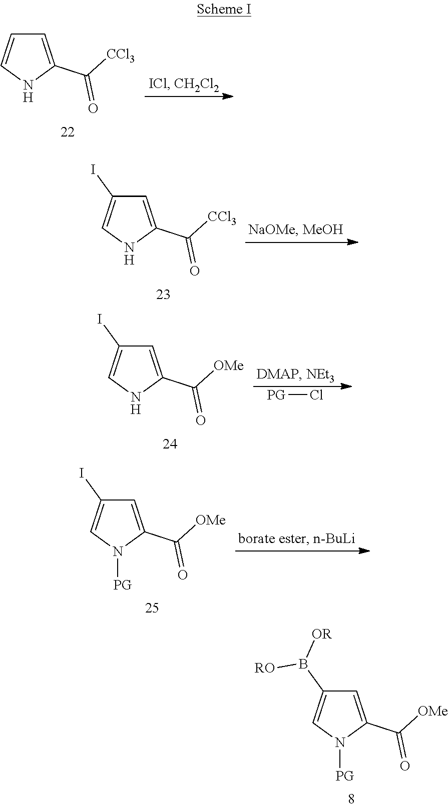

Scheme I above depicts a general method for preparing the compounds as disclosed herein. The pyrrole compound of Formula 22 is iodinated and esterified to form the compound of Formula 23. The pyrrole moiety is optionally protected at the —NH— with a suitable amino protecting group to form the compound of Formula 25. Amino protecting groups are well known in the art and are described in detail in Greene's Protective Groups in Organic Synthesis, 5^{th} Ed. 5, 2014, Theodora W. Greene and Peter G. M. Wuts, published by John Wiley and Sons, the entirety of which is hereby incorporated by reference. The iodo moiety of Formula 25 is replaced by an appropriate boronic acid or ester to afford Formula 8.

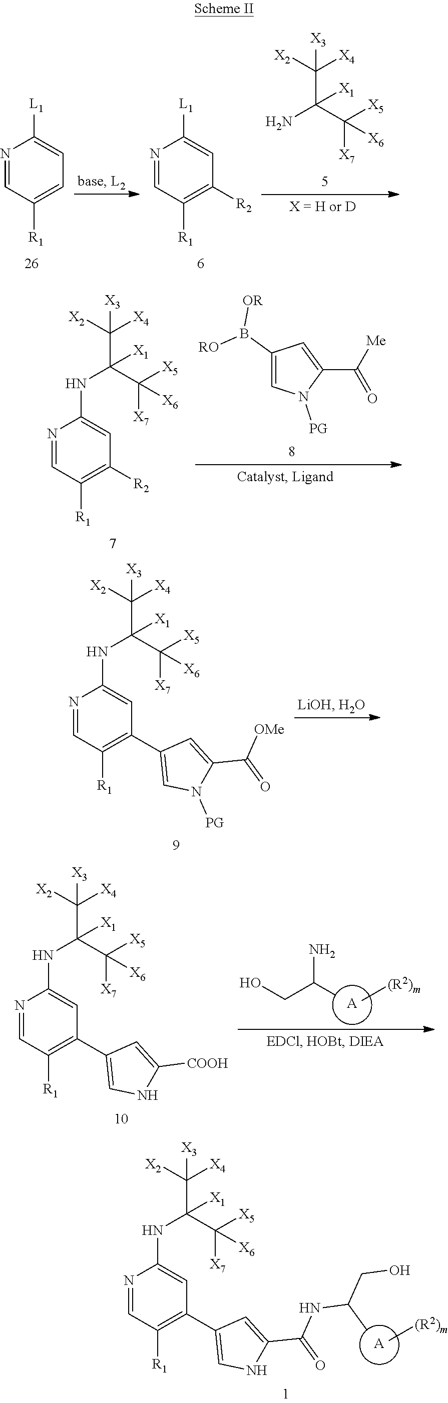

Scheme II depicts a general synthetic route for preparing compounds as disclosed herein. Because the present compounds relate to a multi-substituted pyridine moiety, the sequence of reactions is considered and methods of activating positions on the pyridine are utilized to direct regiochemistry. In the first step, the leaving group L₂ is installed on the pyridine ring in a regioselective manner. In the second step, the leaving group L₁ may be displaced by an alcohol, thiol or amine as desired. Various L₂ leaving groups are amenable to this reaction. Examples of such groups include, but are not limited to, halogens, activated ethers and activated esters. This reaction is followed by the replacement of a second leaving group L₂ through either a metal catalyzed cross-coupling reaction or a nucleophilic aromatic substitution reaction to form the intermediate of formula 9. Various L₂ leaving groups are amenable to this reaction. Examples of such groups include, but are not limited to, halogens and activated ethers, activated esters, boronic acid, boronate ester, or phosphonium salts. The protecting group on the pyrrole is them removed by methods suitable for removing the amino protecting group. Depending on which amino protecting group is used, the conditions suitable for removing it may simultaneously saponify or otherwise provide the carboxylate functional group as depicted in the compound of Formula 10. If the conditions suitable for removing the amino protecting group are not suitable for providing the carboxylate of Formula 10, then another chemical transformation step may be employed. Compounds of formula 1 are prepared from Formula 10 by coupling the resultant carboxylic acid group with a desired amine. A variety of amide bond coupling conditions are useful for said reaction and can include the step of activating the carboxylic acid of the compound of Formula 10 prior to or simultaneously with treatment with the desired amine. Such conditions include, but are not limited to, those described in detail in the Examples section below.

Compounds according to the intermediate of Formula 5 are commercially available or can be synthesized via the following schemes. In the below schemes, the acetone starting material (e.g., D6-acetone) can be obtained from commercial suppliers and can be used in the preparation of, e.g., D6- and D7-isopropylamine. The produced intermediate of Formula 5 can be isolated as a maleate salt (described in detail below in the examples section).

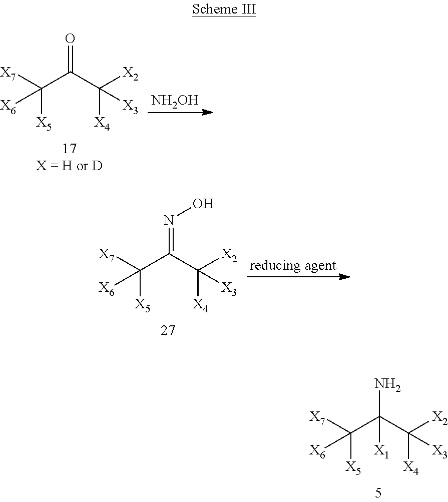

Scheme III depicts a general synthetic route for preparing the intermediate of Formula 5. In the first step, suitable conditions are used for the oximation of acetone with hydroxylamine to provide the intermediate of Formula 27. In the second step, reduction of the oxime to the amine functional group in the intermediate of Formula 5 is accomplished using an appropriate reducing agent. This approach can be used for preparation D1-isopropylamine (X₁=D), D6-isopropylamine (X₂₋₇=D), and D7-isopropylamine (X₁₋₇=D).

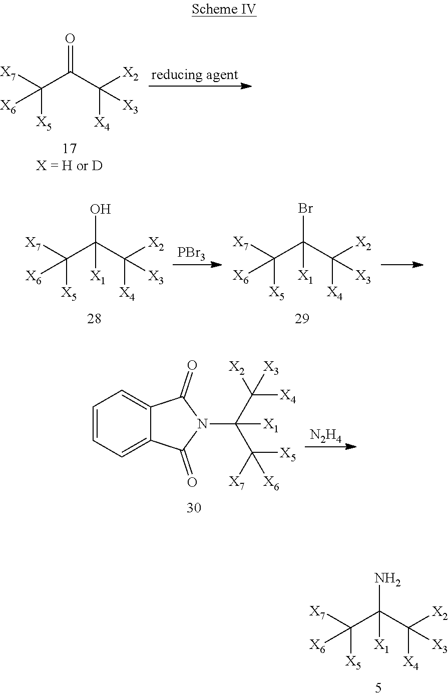

Scheme IV depicts an additional general synthetic route for preparing compounds according to the intermediate of Formula 5. The method according to this scheme is published in the Journal of Labelled Compounds and Radiopharmaceuticals, 2016, 59: 552-556, which is incorporated by reference in its entirety, for the preparation of D6-isopropylamine, but can be extended to the preparation of, inter alia, D7-isopropylamine

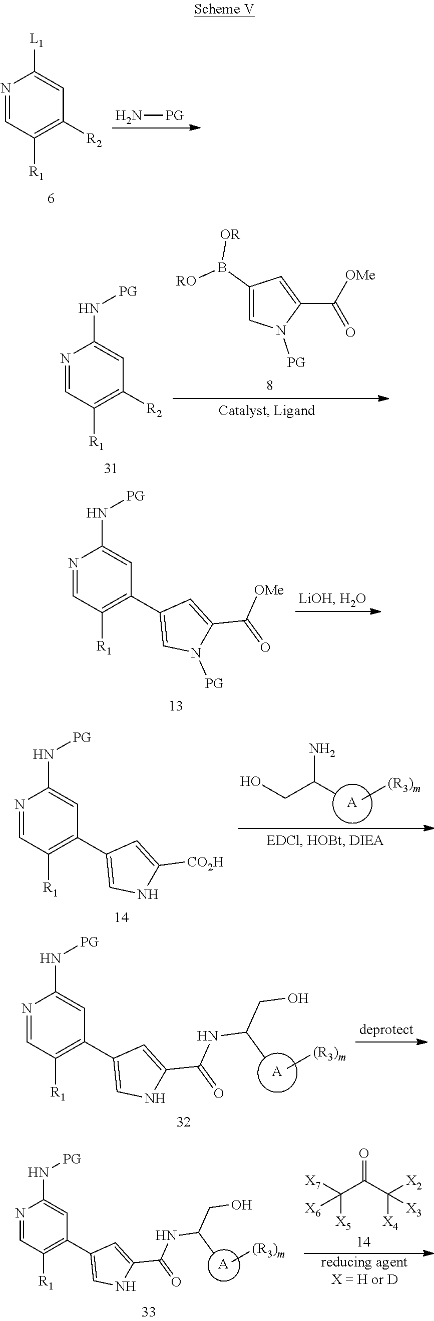

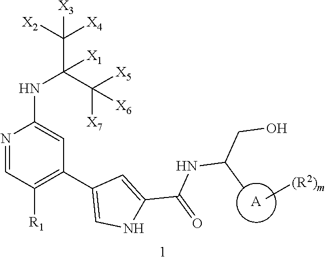

Scheme V above depicts an alternate route to prepare compounds as disclosed herein. The L₁ group of the intermediate of Formula 6 is displaced with the desired amine H₂N-PG to afford the intermediate of Formula 31. Formula 31 may then be utilized to prepare the intermediate of Formula 32 according to conditions that include, but are not limited to, those described in Scheme II, those described in the synthetic examples below, and methods known to one of ordinary skill in the art. The protecting group on the amino pyridine moiety is removed by methods suitable for the deprotection of the amino protecting group used. Reductive amination of the intermediate of Formula 33 with a ketone in the presence of a suitable reducing agent can be utilized to prepare compounds as disclosed herein.

Additional suitable amino protecting groups are well known in the art and include those described in detail in Protecting Groups in Organic Synthesis, Theodora W. Greene and Peter G. M. Wuts, 1991, published by John Wiley and Sons. In some embodiments, the PG group is an alkyl or aryl sulfonyl moiety. Examples of such groups include mesyl, tosyl, nosyl, brosyl, and 2,4,6-trimethylbenzenesulfonyl (“Mts”). Other such groups include Bn, PMB, Ms, Ts, SiR₃, MOM, BOM, Tr, Ac, CO₂R, CH₂OCH₂CH₂Si(CH₃)₃.

A suitable leaving group is a chemical group that is readily displaced by a desired incoming chemical moiety. Thus, the choice of the specific suitable leaving group is predicated upon its ability to be readily displaced by the incoming chemical moiety. Suitable leaving groups are well known in the art, e.g., see, “Advanced Organic Chemistry,” Jerry March, 5th Ed., pp. 351-357, John Wiley and Sons, N.Y. Such leaving groups include, but are not limited to, halogen, alkoxy, sulphonyloxy, optionally substituted alkylsulphonyl, optionally substituted alkenylsulfonyl, optionally substituted arylsulfonyl, and diazonium moieties. Examples of suitable leaving groups include chloro, iodo, bromo, fluoro, methanesulfonyl (mesyl), tosyl, triflate, nitro-phenylsulfonyl (nosyl), and bromo-phenylsulfonyl (brosyl).

### Metabolites of Ulixertinib

According to some embodiments, the compositions disclosed herein have an altered metabolite profile relative to the non-isotopically enriched compound. Some of the metabolites of ulixertinib have been described previously by Bin Yu et al., “Pharmacokinetics and metabolism of ulixertinib in rat by liquid chromatography combined with electrospray ionization tandem mass spectrometry,” Separation Science, vol. 43, issue 7, pages 1275-1283 (2020), which is incorporated by reference in its entirety. Briefly, the metabolites of ulixertinib comprise:

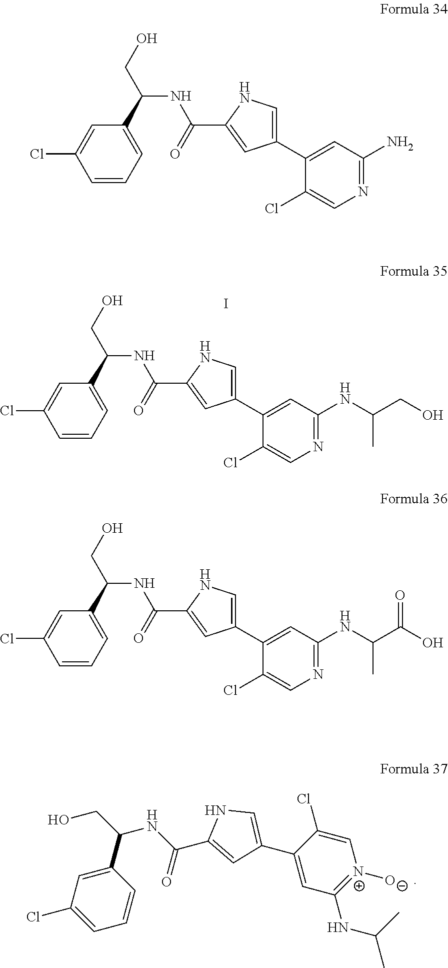

In some embodiments, the metabolites of ulixertinib comprise

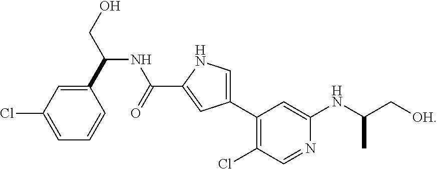 [SMILES: C[C@H](CO)Nc1cc(-c2c[nH]c(C(=O)N[C@H](CO)c3cccc(Cl)c3)c2)c(Cl)cn1]

In some embodiments, the metabolites of ulixertinib comprise

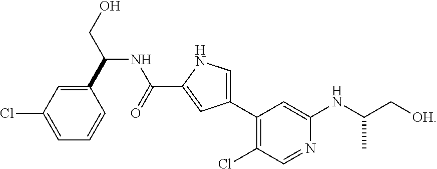 [SMILES: C[C@@H](CO)Nc1cc(-c2c[nH]c(C(=O)N[C@H](CO)c3cccc(Cl)c3)c2)c(Cl)cn1]

In some embodiments, the metabolites of ulixertinib comprise

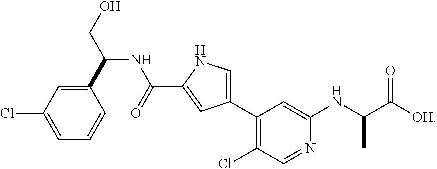 [SMILES: C[C@@H](Nc1cc(-c2c[nH]c(C(=O)N[C@H](CO)c3cccc(Cl)c3)c2)c(Cl)cn1)C(=O)O]

In some embodiments, the metabolites of ulixertinib comprise

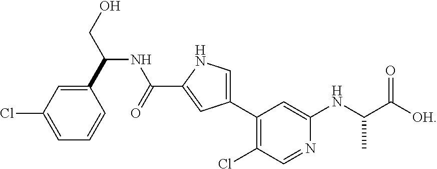 [SMILES: C[C@H](Nc1cc(-c2c[nH]c(C(=O)N[C@H](CO)c3cccc(Cl)c3)c2)c(Cl)cn1)C(=O)O]

The steady state exposure levels of non-isotopically enriched parent ulixertinib and related metabolites in rat (day 5) and human subjects (day 15) is provided in TABLE 1 below:

TABLE 1

| | Steady-State AUC (μg*hr/mL, Mean ± SD) | |
|---|---|---|
| | (% of total Ulixertinib related exposure) | |
| Compound ID | Rat | Human |
| Formula 35 | 1.2 ± 0.2 (0.6%) | 3.4 ± 2.3 (4.3%) |
| Formula 34 | 15.9 ± 4.7 (8.1%) | 39.4 ± 12.0 (49.9%) |
| Formula 37 | 0.4 ± 0.2 (0.2%) | 6.5 ± 5.5 (8.2%) |
| Ulixertinib | 179 ± 49 (91.1%) | 29.6 ± 16.1 (37.5%) |
| Total | 196.5 | 78.9 |
| Formula 34/Ulixertinib | 0.09 | 1.3 |

In some embodiments, the deuterated compounds disclosed herein have reduced formation of one or more metabolites relative to the non-isotopically enriched compound. In some embodiments, deuterated analogs of ulixertinib are effective to reduce formation of the N-des-alkyl metabolite Formula 34. In some embodiments, one or more of Formula 21, Formula 19, and Formula 18 are effective to reduce formation of N-des-alkyl metabolite Formula 34. See, e.g., Tables 6 (HLM) and 8 (rat plasma). In some embodiments, deuterated analogs of ulixertinib are effective to reduce formation of the i-Propyl Hydroxyl Metabolite Formula 35. In some embodiments, one or more of BVD-523-D1, BVD-523-D6, and BVD-523-D7 are effective to reduce formation of the i-Propyl Hydroxyl Metabolite Formula 35. See, e.g., Tables 6 (HLM) and 8 (rat plasma). In some embodiments, one or more of Formula 21, Formula 19, and Formula 18 are effective to reduce formation of the carboxylic acid metabolite Formula 36. See, e.g., Table 6 (HLM).

In some embodiments, the deuterated compounds disclosed herein have an increased formation, reduced formation, or the same formation, of one or more metabolites relative to the non-isotopically enriched compound. In some embodiments, the deuterated analogs of ulixertinib are effective to increase formation, reduce formation, or maintain the same formation, of the N-oxide metabolite Formula 37. See, e.g., Tables 6 (HLM) and 8 (rat plasma).

In some embodiments, the metabolism of the deuterated compounds disclosed herein is altered relative to the non-isotopically enriched compounds via inhibition and/or induction of the polymorphically expressed cytochrome P450 metabolic enzymes. In some embodiments, the deuterated compounds disclosed herein are effective to induce and/or inhibit one or more of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3ASP1, CYPa5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51. In some embodiments, the deuterated compounds disclosed herein are effective to induce and/or inhibit one or more of CYP3A4, CYP3A5, CYP2C8, CYP2C9, CYP2D6, CYP2C19, CYP1A2, CYP2B6, and CYP2E1. In some embodiments, the deuterated compounds disclosed herein are effective to decrease the metabolism of the deuterated compound via inhibition of one of more of the cytochrome P450 enzymes disclosed herein. In some embodiments, the deuterated compounds disclosed herein are effective to inhibit the metabolism of a concomitantly administered drug.

### Uses, Formulations and Administration

As discussed above, the present disclosure provides compounds that are inhibitors of protein kinases, and thus the present compounds are useful for the treatment of diseases, disorders, and conditions including, but not limited to cancer, autoimmune disorders, neurodegenerative and neurological disorders, schizophrenia, bone-related disorders, liver disease, and cardiac disorders. Accordingly, in another aspect of the present disclosure, pharmaceutically acceptable compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of the present disclosure can exist in free form for treatment, or where appropriate, as a pharmaceutically acceptable derivative thereof. According to the present disclosure, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound disclosed herein that, upon administration to a recipient, is capable of providing, either directly or indirectly, a compound disclosed herein or an inhibitorily active metabolite or residue thereof. As used herein, the term “inhibitorily active metabolite or residue thereof” means that a metabolite or residue thereof is also an inhibitor of ERK2 protein kinase.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds disclosed herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺ (C₁₋₄ alkyl)₄ salts. This disclosure also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described above, the pharmaceutically acceptable compositions disclosed herein additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds disclosed herein, such as by producing any undesirable biological effect or otherwise interacting in a deleterious manner with any other component(s) of the pharmaceutically acceptable composition, its use is contemplated to be within the scope of this disclosure. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Uses of Compounds and Pharmaceutically Acceptable Compositions

In yet another aspect, a method for the treatment or lessening the severity of cancer, an autoimmune disorder, a neurodegenerative or neurological disorder, liver disease, or a cardiac disorder is provided comprising administering an effective amount of a compound disclosed herein, or a pharmaceutically acceptable composition comprising a compound disclosed herein to a subject in need thereof. In certain embodiments of the present disclosure an “effective amount” of the compound or pharmaceutically acceptable composition is that amount effective for treating or lessening the severity of a disease, condition, or disorder selected from cancer, an autoimmune disorder, a neurodegenerative or neurological disorder, schizophrenia, a bone-related disorder, liver disease, or a cardiac disorder. The compounds and compositions, according to the method disclosed herein, may be administered using any amount and any route of administration effective for treating or lessening the severity of cancer, an autoimmune disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. The compounds disclosed herein are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions disclosed herein will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions disclosed herein can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds disclosed herein may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound disclosed herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds disclosed herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound disclosed herein include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this disclosure. Additionally, the present disclosure provides the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds disclosed herein are useful as inhibitors of ERK protein kinases. In one embodiment, the compounds and compositions disclosed herein are inhibitors of one or both of ERK1 and ERK2 protein kinases and thus, without wishing to be bound by any particular theory, the compounds and compositions are particularly useful for treating or lessening the severity of a disease, condition, or disorder where activation of one or both of ERK1 and ERK2 protein kinases is implicated in the disease, condition, or disorder. When activation of ERK1 and/or ERK2 protein kinases is implicated in a particular disease, condition, or disorder, the disease, condition, or disorder may also be referred to as “ERK1- or ERK2-mediated disease”, condition, or disease symptom. Accordingly, in another aspect, the present disclosure provides a method for treating or lessening the severity of a disease, condition, or disorder where activation of one or both of ERK1 and ERK2 protein kinases is implicated in said disease, condition, or disorder.

The activity of an inhibitor of ERK1 and/or ERK2 protein kinases may be assayed in vitro, in vivo or in a cell line. In vitro assays include assays that determine inhibition of either the phosphorylation activity or ATPase activity of activated ERK1 or ERK2 protein kinases. Alternate in vitro assays quantitate the ability of the inhibitor to bind to ERK1 or ERK2 protein kinases. Inhibitor binding may be measured by radiolabelling the inhibitor prior to binding, isolating the inhibitor/ERK1 or inhibitor/ERK2 complex and determining the amount of radiolabel bound. Alternatively, inhibitor binding may be determined by running a competition experiment where new inhibitors are incubated with ERK1 or ERK2 protein kinases bound to known radioligands.

The term “measurably inhibit”, as used herein means a measurable change in ERK1 or ERK2 protein kinase activity between a sample comprising said composition and a ERK1 or ERK2 protein kinase and an equivalent sample comprising ERK1 or ERK2 protein kinase in the absence of said composition. Such measurements of protein kinase activity are known to one of ordinary skill in the art and include those methods set forth herein below.

According to another embodiment, the present disclosure provides a method of inhibiting ERK1 or ERK2 protein kinase activity in a patient comprising the step of administering to said patient a compound disclosed herein, or a composition comprising said compound.

The term “ERK-mediated condition” or “disease”, as used herein, means any disease or other deleterious condition in which ERK is known to play a role. The term “ERK-mediated condition” or “disease” also means those diseases or conditions that are alleviated by treatment with an ERK inhibitor. Such conditions include, without limitation, cancer, stroke, diabetes, hepatomegaly, cardiovascular disease including cardiomegaly, Alzheimer's disease, cystic fibrosis, viral disease, autoimmune diseases, atherosclerosis, restenosis, psoriasis, allergic disorders including asthma, inflammation, neurological disorders and hormone-related diseases. The term “cancer” includes, but is not limited to the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia.

Accordingly, another embodiment the present disclosure provides treating or lessening the severity of one or more diseases in which ERK is known to play a role. Specifically, the present disclosure provides a method of treating or lessening the severity of a disease or condition selected from cancer, stroke, diabetes, hepatomegaly, cardiovascular disease including cardiomegaly, Alzheimer's disease, cystic fibrosis, viral disease, autoimmune diseases, atherosclerosis, restenosis, psoriasis, allergic disorders including asthma, inflammation, neurological disorders and hormone-related diseases, wherein said method comprises administering to a patient in need thereof a composition according to the methods disclosed herein.

According to another embodiment, the present disclosure provides a method of treating a cancer selected from breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon-rectum, large intestine, rectum, brain and central nervous system, and leukemia.

Another embodiment relates to a method of treating melanoma, breast cancer, colon cancer, or pancreatic cancer in a patient in need thereof.

It will also be appreciated that the compounds and pharmaceutically acceptable compositions disclosed herein can be employed in combination therapies, that is, the compounds and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a compound disclosed herein may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”.

For example, chemotherapeutic agents or other anti-proliferative agents may be combined with the compounds disclosed herein to treat proliferative diseases and cancer. Examples of known chemotherapeutic agents include, but are not limited to, For example, other therapies or anticancer agents that may be used in combination with the anticancer agents disclosed herein include surgery, radiotherapy (in but a few examples, gamma.-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), Gleevec™, adriamycin, dexamethasone, and cyclophosphamide. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

Other examples of agents the inhibitors may also be combined with include, without limitation: treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions disclosed herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In an alternate embodiment, the methods disclosed herein that utilize compositions that do not contain an additional therapeutic agent, comprise the additional step of separately administering to said patient an additional therapeutic agent. When these additional therapeutic agents are administered separately they may be administered to the patient prior to, sequentially with or following administration of the compositions disclosed herein.

The compounds disclosed herein or pharmaceutically acceptable compositions thereof may also be incorporated into compositions for coating implantable medical devices, such as prostheses, artificial valves, vascular grafts, stents and catheters. Accordingly, the present disclosure provides, in another aspect, a composition for coating an implantable device comprising a compound as described generally above, and in classes and subclasses herein, and a carrier suitable for coating said implantable device. In still another aspect, the present disclosure provides an implantable device coated with a composition comprising a compound as described generally above, and in classes and subclasses herein, and a carrier suitable for coating said implantable device.

Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

Another aspect of the present disclosure relates to inhibiting ERK1 or ERK2 protein kinase activity in a biological sample or a patient, which method comprises administering to the patient, or contacting said biological sample with a compound disclosed herein or a composition comprising said compound. The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of ERK1 or ERK2 protein kinase activity in a biological sample is useful for a variety of purposes that are known to one of skill in the art. Examples of such purposes include, but are not limited to, blood transfusion, organ-transplantation, biological specimen storage, and biological assays.

The terminology used herein is for the purpose of describing particular embodiments only and is not intended to be limiting. As used in the specification and the appended claims, the singular forms “a,” “an,” and “the” include plural referents unless the context clearly dictates otherwise.

The following examples are provided to further illustrate the methods of the present disclosure. These examples are illustrative only and are not intended to limit the scope of the disclosure in any way.

### EXAMPLES

### Example 1

D7-Ulixertinib was prepared as follows:

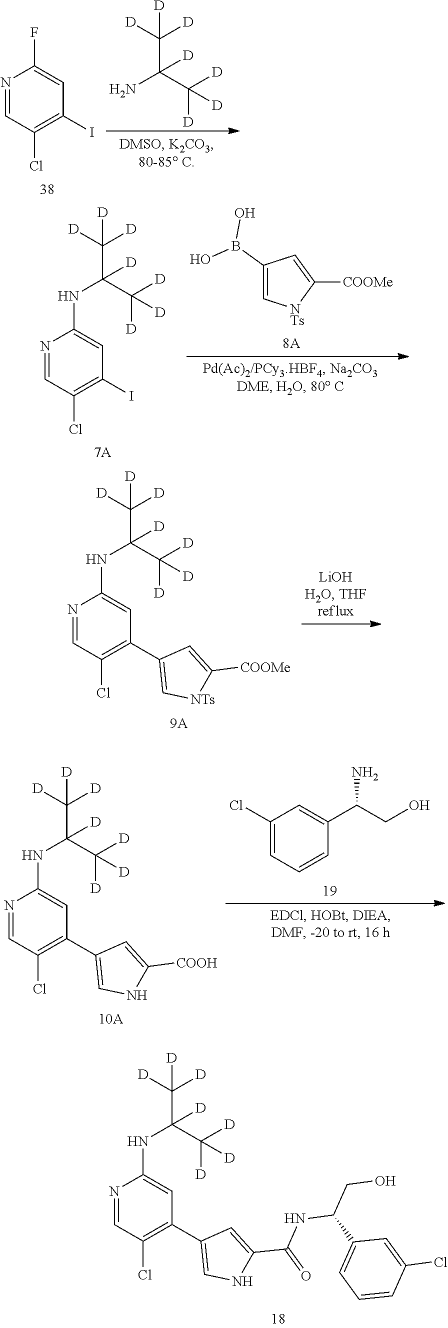

### Preparation of Intermediate Formula 7A

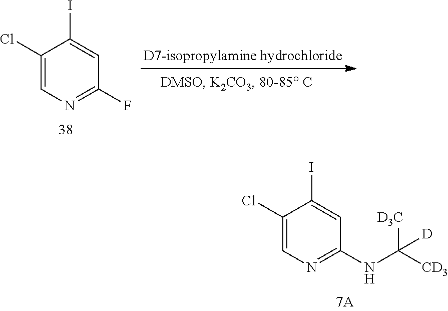 [SMILES: Fc1cc(I)c(Cl)cn1 ; [2H]C([2H])([2H])C([2H])(Nc1cc(I)c(Cl)cn1)C([2H])([2H])[2H]]

To the flask were added DMSO (128 mL, 6V), Formula 38 (21.37 g, 1.0 equiv.), d7-isopropylamine hydrochloride (12.72 g, 1.5 equiv.) and K₂CO₃ (25.84 g, 2.25 equiv.) at ambient temperature. The temperature of the mixture was increased to 80 to 85° C. and stirred until reaction completion. The temperature of the mixture was decreased to 20 to 25° C. and water (770 mL, 36V) was added to the mixture. The mixture was extracted with EtOAc (427 mL, 20V×3), and the organic phases were combined. The EtOAc solution was washed with water (427 mL, 20V×2), and brine (213 mL, 10V×1). The solution was then solvent-swapped into n-heptane (214 mL, 10V×2) to a final volume of 6. The temperature of the n-heptane mixture was reduced to 0 to 10° C. The slurry was filtered and the isolated solids dried to give 16 g of product (Formula 7A) as an off-white solid. Yield: 77.8%, HPLC purity: 89.2% (215 nm); LC-MS: m/z 304.0 (M+1); ¹H-NMR (400 MHz, CDCl₃): δ 8.01 (s, 1H), 6.88 (s, 1H).

### Preparation of Intermediate Formula 9A

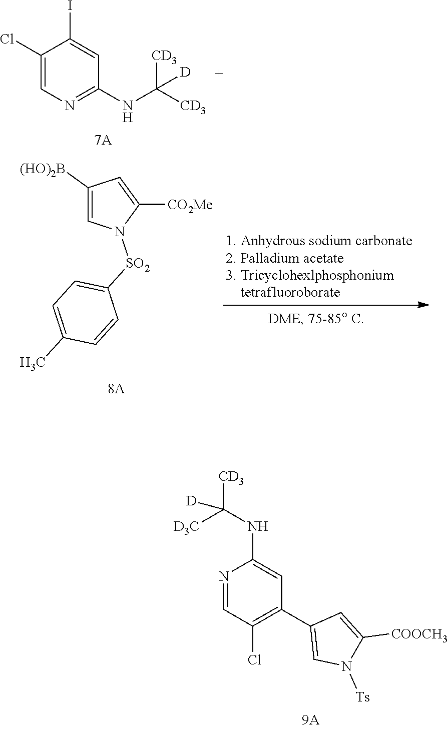 [SMILES: [2H]C([2H])([2H])C([2H])(Nc1cc(I)c(Cl)cn1)C([2H])([2H])[2H] ; COC(=O)c1cc(B(O)O)cn1S(=O)(=O)c1ccc(C)cc1 ; [2H]C([2H])([2H])C([2H])(Nc1cc(-c2cc(C(=O)OC)n(S(=O)(=O)c3ccc(C)cc3)c2)c(Cl)cn1)C([2H])([2H])[2H]]

To a solution of Formula 7A (1.0 g. 3.29 mmol, 1 equiv.) and Formula 8A (1.28 g, 3.95 mmol, 1.2 equiv.) in DME (15 mL, 15V) and water (1 mL, 1V), was added palladium acetate (0.01 g, 0.45 mmol, 0.14 equiv.), tricyclochexylphosphonium tetrafluoroborate (0.01 g, 0.45 mmol, 0.14 equiv.) and sodium carbonate (0.52 g, 4.9 mmol, 1.5 equiv.) under nitrogen. The temperature of the mixture was raised to 75 to 85° C. and stirred for 12 hr. The reaction was cooled to 20-25° C. and the mixture was filtered. The filtrate was collected and concentrated to give a residue that was re-slurried with 2.8 mL methanol. The methanol slurry was filtered and the filter cake was washed with methanol (1 mL×2). The solids were dried under nitrogen to give 1.1 g of product (Formula 9A) as an off-white solid. Yield: 79.0%, HPLC purity: 99.6% (215 nm); LC-MS: m/z 455.1 (M+1); ¹H-NMR (400 MHz, DMSO-d6): δ 8.35 (s, 1H), 8.31 (s, 1H), 8.13-8.16 (d, 2H), 7.49-7.52 (m, 3H), 6.57 (s, 1H), 4.56 (s, 1H), 3.92 (s, 3H), 2.61 (s, 3H).

### Preparation of Intermediate Formula 10A

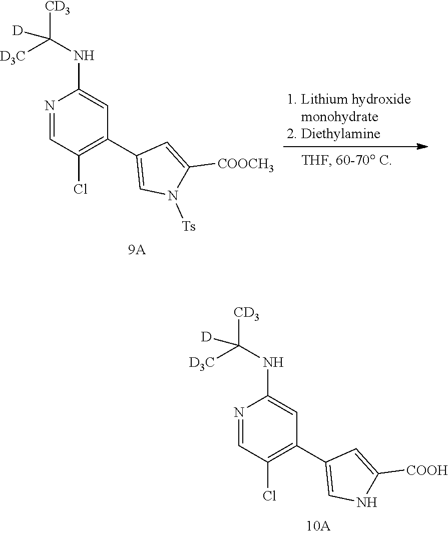 [SMILES: [2H]C([2H])([2H])C([2H])(Nc1cc(-c2cc(C(=O)OC)n(S(=O)(=O)c3ccc(C)cc3)c2)c(Cl)cn1)C([2H])([2H])[2H] ; [2H]C([2H])([2H])C([2H])(Nc1cc(-c2c[nH]c(C(=O)O)c2)c(Cl)cn1)C([2H])([2H])[2H]]

An aqueous solution of LiOH (5.0 equiv., 7 wt. %) and diethylamine (7.26 g, 2.0 equiv.) were added to a solution of Formula 9A (22 g, 1.0 equiv.) in THF (4V) at 15 to 30° C. The temperature of the mixture was increased to 60 to 70° C. until reaction completion. The temperature of the reaction was decreased to 20 to 25° C. The solution was concentrated under vacuum and pH was adjusted to 6-7 with 1N HCl. The solids were collected by filtration and dried under vacuum to give 7.2 g of product (Formula 10A) as an off-white solid. Yield: 64.9%, HPLC purity: 98.9% (215 nm); LC-MS: m/z 287.1 (M+1); ¹H-NMR (400 MHz, DMSO-d6): δ 12.17 (s, 1H), 7.98 (s, 1H), 7.96 (s, 1H), 7.46 (s, 1H), 7.18 (s, 1H), 6.59 (s, 1H), 5.76 (s, 1H).

### Preparation of the Compound of Formula 18

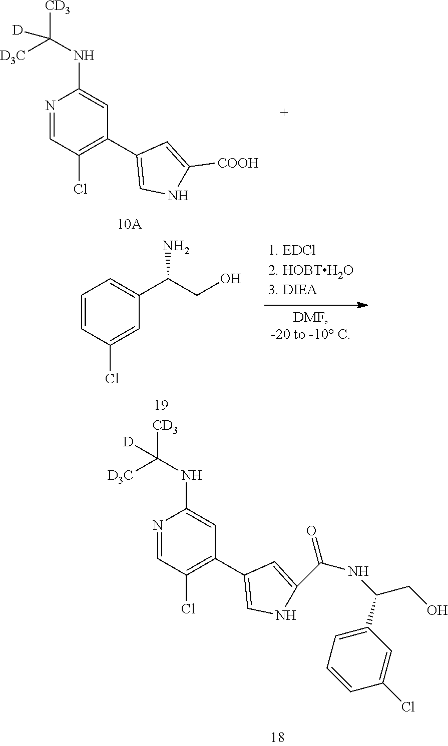 [SMILES: [2H]C([2H])([2H])C([2H])(Nc1cc(-c2c[nH]c(C(=O)O)c2)c(Cl)cn1)C([2H])([2H])[2H] ; N[C@H](CO)c1cccc(Cl)c1 ; [2H]C([2H])([2H])C([2H])(Nc1cc(-c2c[nH]c(C(=O)N[C@H](CO)c3cccc(Cl)c3)c2)c(Cl)cn1)C([2H])([2H])[2H]]

HOBt (1.2 equiv., 5.21 g) was added to a solution of Formula 10A (1.0 equiv., 8 g) in DMF (1.5V, 12 mL) at −10 to −20° C. followed by EDCI (1.1 equiv., 5.94 g). The temperature of the mixture was decreased to −20 to −30° C. and Formula 19 (1.2 equiv., 5.84 g) and DIPEA (1.2 equiv. 0.31 g) were added to the reaction mixture. The mixture was stirred at 0 to 10° C. until reaction completion. Ethyl acetate (10V, 80 mL) and water (4V, 32 mL) were added to the reaction mixture. The phases was separated and the aqueous phase was extracted with ethyl acetate (10V×3, 80 mL). The combined organic phases were washed sequentially with water (4V×2, 32 mL each), 5% aqueous acetic acid (7V×2), aqueous Na₂CO₃ (7V×2, 17 wt. %) and brine (10V×2). Silica (1 g/g, 8 g) was charged to the organic solution and the silica slurry was stirred for 2 to 4 hr and filtered. The filter cake was rinsed with ethyl acetate (4.5 g/g, 36 mL). The combined filtrate was concentrated under vacuum to 1 to 2V and MTBE (1.48 g/g, 11.8 g) was then added. The temperature of the mixture was decreased to 0° C. and stirred for 2 hr. The solids were collected by filtration and dried to give 9.1 g (Formula 18) as an off-white solid. Yield: 69.7%, HPLC purity: 99.0% (215 nm); HPLC chiral purity (ee): 100.0% (254 nm); LC-MS: m/z 440.1 (M+1); ¹H-NMR (400 MHz, CD₃OD): δ 7.99 (s, 1H), 7.48-7.52 (m, 3H), 7.39 (s, 1H), 7.27-7.31 (m, 2H), 7.23-7.26 (m, 2H), 6.55 (s, 1H), 5.15 (q, 1H), 3.85 (m, 2H).

The compound of Formula 18 was further reacted with HCl to produce the HCl salt Formula 18A. At 15 to 25° C., filtered anhydrous ethanol (0.26V), filtered methanol (0.02V), and filtered isopropanol (0.02V) were charged into a glass flask and stirred for 20 to 30 min. Then hydrogen chloride gas was bubbled into the mixture under stirring at 10 to 25° C. After 2 hr, the mixture was sampled and analyzed every 2 to 4 hr until the content of hydrogen chloride reached ≥35% by wt. While maintaining the temperature at 15 to 25° C., filtered anhydrous ethanol (9V), filtered methanol (0.5V), and filtered isopropanol (0.5V) were charged into a separate reactor through an in-line fluid filter and stirred for 20 to 30 min. Then Formula 18 (1.0 equiv.) was added. The temperature of the mixture was increased to 70 to 75° C. at the rate of 15 to 25° C./hr and stirred until the solids were completely dissolved. At 70 to 75° C., seed crystals (0.1 wt %) were added to the mixture, and then the anhydrous HCl solution was added. The mixture was maintained at 70 to 75° C. for 1 to 2 hr with stirring. The temperature of the mixture was decreased to 15 to 25° C. at the rate of 5 to 15° C./hr and then maintained at 15 to 25° C. for 4 to 6 hr under stirring. The mixture was filtered and the filter cake was rinsed with filtered MTBE. The filter cake was dried under nitrogen at 40 to 50° C. for 24 hr to afford 8.29 g of product (Formula 18A) as a white solid. Yield: 84.5%, HPLC purity: 99.4% (215 nm); HPLC chiral purity (ee): 100.0% (254 nm); HPLC assay purity: 99% (215 nm); Chloride content: 7.31%; Isotopic purity: 99.7% (CD₃), 100% (CD); LC-MS: m/z 440.2 (M+1); ¹H-NMR (400 MHz, CD₃OD): δ 7.99 (s, 1H), 7.75 (s, 1H), 7.48 (s, 1H), 7.42 (s, 1H), 7.39 (s, 1H), 7.27-7.31 (m, 2H), 7.08 (s, 1H), 5.11 (q, 1H), 3.88 (m, 2H).

### Example 2

Formula 19 was prepared as follows:

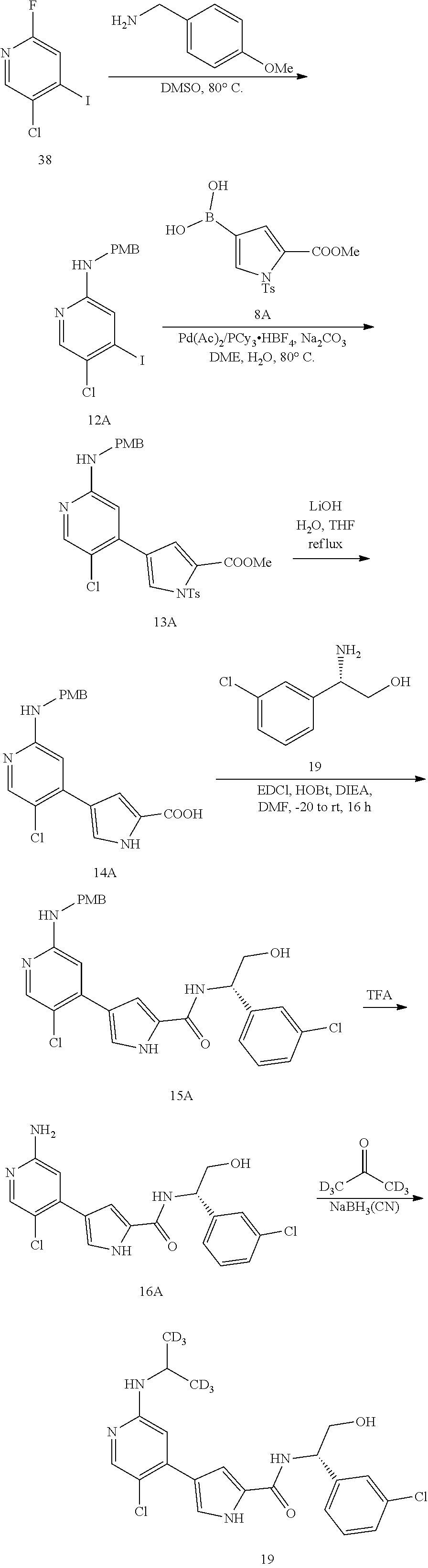

### Preparation of Formula 12A

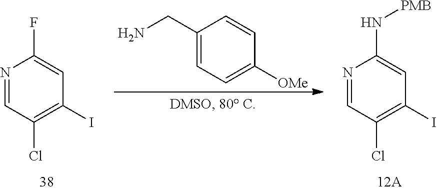

Formula 38 (200 g, 777 mmol, 1 equiv.), p-methoxybenzylamine (320 g, 2.33 mol, 3 equiv.) and DMSO (2500 mL) were added to a flask. The temperature of the mixture was increased to 80° C. and stirred for 12 hr. Reaction mixture temperature was decreased to 20 to 25° C. The reaction was partitioned between water (4 L) and DCM (4 L). Water (4.5 L) was added to the organic layer, and the pH was adjusted to pH 3 to 4 with stirring. Stirring was stopped and the aqueous layer was separated and discarded. Water (4.5 L) was added to the DCM layer and the pH was adjusted to 9. The mixture was stirred for 5 min. Stirring was stopped and the aqueous layer was separated and discarded. The organic layer was dried and concentrated under reduced pressure to afford 240 g product (Formula 12A) as a white solid. Yield: 82.5%, HPLC purity: 97.3% (254 nm); LC-MS: m/z 375.0 (M+1); ¹H-NMR (400 MHz, DMSO-d₆): δ 7.99 (s, 1H), 7.28-7.31 (t, 1H), 7.21-7.23 (d, 2H), 7.11 (s, 1H), 6.85-6.88 (d, 2H), 4.35-4.36 (d, 2H), 3.73 (s, 3H).

### Preparation of Formula 13A

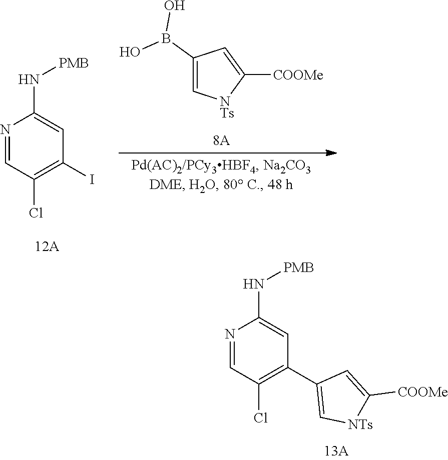

Formula 12A (143 g, 380 mmol, 1 equiv.), Formula 8A (123 g, 380 mmol, 1.0 equiv.), Pd(OAc)₂ (2.56 g, 11.4 mmol, 0.03 equiv.), PCy₃·HBF₄ (5.60 g, 15.2 mmol, 0.04 equiv.), sodium carbonate (60.42 g, 570 mmol, 1.5 equiv.), DME (1500 mL, 10V) and water (150 mL, 1V). The temperature of the reaction mixture was raised to 80° C. under nitrogen and stirred for 23 hr at 80° C. An additional quantity of Formula 8A (32 g) was added to flask and the reaction mixture was stirred overnight. The mixture was filtered on celite pad and the filtrate was collected and concentrated under reduced pressure. Methanol (750 ml) was added to the residue and the slurry was stirred for 2 hr, and filtered to afford 180 g of product (Formula 13A) as an off-white solid. Yield: 89.6%, HPLC purity: 90.5% (254 nm); LC-MS: m/z 526.0 (M+1); ¹H-NMR (400 MHz, DMSO-d₆): δ 8.22 (d, 1H), 8.05 (s, 1H), 7.95-7.97 (d, 2H), 7.48-7.50 (d, 2H), 7.24-7.26 (d, 2H), 7.14-7.17 (t, 1H), 6.86-6.88 (d, 2H), 6.76 (s, 1H), 4.41-4.42 (d, 2H), 3.69 (s, 6H), 2.42 (s, 3H).

### Preparation of Formula 14A

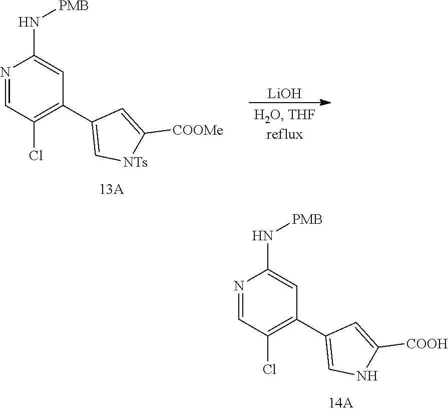

Formula 13A (170 g, 323 mmol, 1 equiv.), lithium hydroxide monohydrate (67.8 g, 1616 mmol, 5 equiv.), THF (1700 mL, 10V), and water (1500 mL, 8.8V) were added to a flask. The temperature of the mixture was raised to 60 to 80° C. and stirred at that temperature for about 60 hr. The temperature of the reaction mixture was reduced to 20-25° C. The pH of the mixture was adjusted to 3. The organic layer was separated, and the solvent was concentrated under reduced pressure to about 0.33V. The slurry was filtered and dried to give 196 g of product (Formula 14A) as a light yellow solid. The solid was used in the next step without further purification. HPLC purity: 82.5% (254 nm); LC-MS: m/z 358.1 (M+1); ¹H-NMR (400 MHz, DMSO-d₆): δ 12.44 (s, 1H), 8.11 (s, 1H), 7.62 (s, 1H), 7.46-7.48 (d, 2H), 7.26-7.28 (d, 2H), 7.19 (s, 1H), 7.09-7.11 (d, 2H), 7.01 (s, 1H), 6.90-6.92 (d, 2H), 4.50 (s, 2H), 2.23 (s, 3H).

### Preparation of Formula 15A

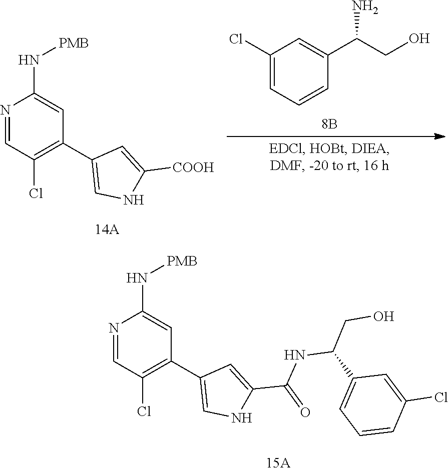

EDCI HCl (132 g, 690 mmol, 1.3 equiv.), HOBt (107 g, 795 mmol, 1.5 equiv.) and DIEA (103 g, 795 mmol, 1.5 equiv.) were added to a solution of Formula 14A (190 g, 530 mmol, 1 equiv.) in dry DMF (1330 mL) at −10 to −20° C. The mixture was stirred for 20 min, and then compound Formula 8B (109 g, 640 mmol, 1.2 equiv.) was added to the mixture. The temperature of the reaction mixture was increased to 20 to 25° C. and stirred for 16 hr. The mixture was partitioned between EtOAc (1 L) and water (1 L). The aqueous phase was extracted with EtOAc (3 L) twice. The combined organic layers were washed with water (2 L), brine (2 L), and dried over magnesium sulphate. The mixture was filtered and the filtrate concentrated under reduced pressure. The residue was purified by column chromatography to afford 173 g of product (Formula 15A) as a yellow solid. Yield (two steps):100%; HPLC purity: 97% (254 nm); LC-MS: m/z 511.1 (M+1); ¹H-NMR (400 MHz, DMSO-d₆): δ 11.86 (s, 1H), 8.47 (d, 1H), 7.91 (s, 1H), 7.45 (s, 1H), 7.38-7.30 (m, 5H), 7.24-7.26 (m, 2H), 7.08-7.10 (m, 1H), 6.86-6.88 (m, 2H), 6.62 (s, 1H), 5.02 (m, 2H), 4.40-4.41 (m, 2H), 3.71 (m, 3H), 3.66-3.69 (m, 2H);

### Preparation of Formula 16A

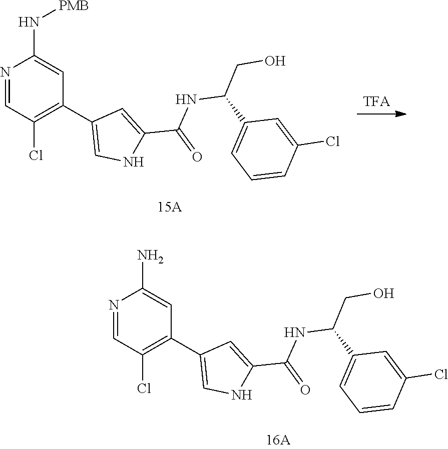

Anisole (25.4 g, 235 mmol) was charged to a solution of Formula 15A (80 g, 156 mmol) in TFA (480 mL) at 20 to 25° C. The mixture was stirred at 50° C. for 14 hr and then the temperature was decreased to 20 to 25° C. The solution was concentrated under reduced pressure. The residue was dissolved in water, and the solution was adjusted to a pH of 8 with an aqueous solution of saturated NaHCO₃, EtOAc (800 ml, 10 vol.) was added to the aqueous solution and the mixture was stirred. The solids were filtered, and recrystallized with EtOAc (50 vol.) to afford 38 g of product (Formula 16A) as a white solid. Yield: 61.5%, HPLC purity: 96.9% (254 nm): HPLC chiral purity(ee) 99.9%, LC-MS m/z 391.0 (M+1); ¹H-NMR (400 MHz, DMSO-d₆): δ 11.86 (s, 1H), 8.47 (d, 1H), 7.91 (s, 1H), 7.45 (s, 1H), 7.38-7.30 (m, 5H), 6.62 (s, 1H), 6.01 (s, 2H), 5.02 (m, 2H), 3.67 (m, 2H).

### Preparation of Compound 118217 (BVD-523-D6)

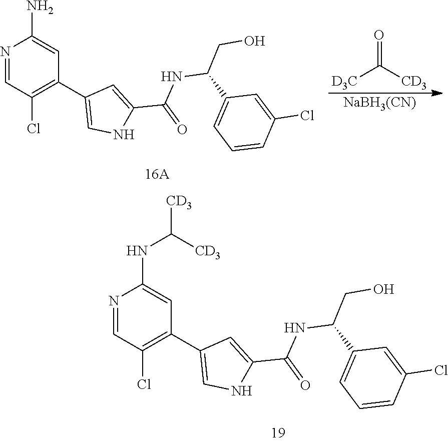

A mixture of Formula 16A (20 g, 52.1 mmol) and Acetone-d6 (80 mL) in buffer (2000 mL, NaOAc: 48 g, AcOH: 68 mL, MeOH: 2000 mL) was stirred at 20 to 25° C. for 1 hr. Then NaBH₃(CN) (48.3 g, 768 mmol) was added to the solution. The mixture was stirred at 55° C. for 18 hr and then decreased to 20 to 25° C. The buffer (400 mL) and Acetone-d6 (30 mL) was added. The mixture was stirred at 20 to 25° C. for 1 hr, and then additional NaBH₃(CN) (16 g) was added. The temperature of the mixture was raised to 55° C. and stirred for an additional 14 hr. The temperature of the mixture was reduced to 20 to 25° C. and washed with an aqueous solution of saturated NaHCO₃. The mixture was concentrated under reduced pressure, and diluted with 2-MeTHF (1500 mL, 75 vol). The solution was washed with an aqueous solution of saturated NaHCO₃ and brine. The organic solution was dried over Na₂SO₄, filtered and the filtrate was concentrated under reduced pressure. The residue was purified by column chromatography and with the crude solids were recrystallized (MeOH:H₂O=2.4:1, 96 ml:40 ml) to afford 14.14 g of final product (Formula 19) as a white solid. Yield: 63%, HPLC purity: 98.2% (254 nm); HPLC chiral purity (ee): 99.9%; LC-MS: m/z 439.1 (M+1); ¹H-NMR: (400 MHz, DMSO-d₆): δ 11.87 (s, 1H), 8.45 (d, 1H), 7.98 (s, 1H), 7.44-7.29 (m, 6H), 6.59 (s, 1H), 6.40-6.39 (m, 1H), 5.10-5.04 (m, 2H), 3.92 (s, 1H), 3.69-3.67 (m, 2H).

The compound of Formula 19 was further reacted with HCl to produce the HCl salt form Formula 19A. At 15 to 25° C., filtered anhydrous ethanol (145 g, 0.26V), filtered methanol (8.1 g, 0.02V), and filtered isopropanol (8.1 g, 0.02V) were charged into a 20 L glass flask and stirred for 20 to 30 min. Then hydrogen chloride gas was bubbled into the mixture under stirring at 10 to 25° C. After 2 hr, the mixture was sampled and analyzed every 2 to 4 hr until the content of hydrogen chloride reached ≥35% by weight. While maintaining the temperature at 15 to 25° C., filtered anhydrous ethanol (4292 g, 9V), filtered methanol (232 g, 0.5V), and filtered isopropanol (232 g, 0.5V) were charged into a separate 20 L reactor through an in-line fluid filter and stirred for 20 to 30 min. Then Formula 19 (580 g, 1.0 equiv.) was added. The temperature of the mixture was increased to 70 and 75° C. at the rate of 15 to 25° C./hr and stirred until the solids were completely dissolved. At 70 to 75° C., seed crystals (0.58 g, 0.1 wt. %) were added to the mixture, and then the anhydrous HCl solution (141 g) was added. The mixture was maintained at 70 to 75° C. for 1 to 2 hr with stirring. The temperature of the mixture was decreased to 15 to 25° C. at the rate of 5 to 15° C./hr and then maintained at 15 to 25° C. for 4 to 6 hr under stirring. The mixture was filtered and the filter cake was rinsed for 20 to 30 min with filtered MTBE. The filter cake was dried under nitrogen at 40 to 50° C. for 24 hr to afford 12.9 g of product (Formula 19A) as a white solid. Yield: 84.5%, HPLC purity: 99.4% (215 nm); HPLC chiral purity (ee): 100.0% (254 nm); HPLC assay purity: 98.3% (215 nm); Chloride content: 7.2%; Isotopic purity: 97.9%; LC-MS: m/z 439.2 (M+1); ¹H-NMR (400 MHz, DMSO-d₆): δ 8.10 (s, 1H), 7.65 (s, 1H), 7.56 (s, 1H), 7.46 (s, 1H), 7.36 (m, 2H), 7.32 (m, 1H), 7.10 (s, 1H), 5.05 (q, 1H), 3.97 (s, 1H), 3.73-3.69 (m, 2H).

### Example 3

Formula 21 was prepared as follows:

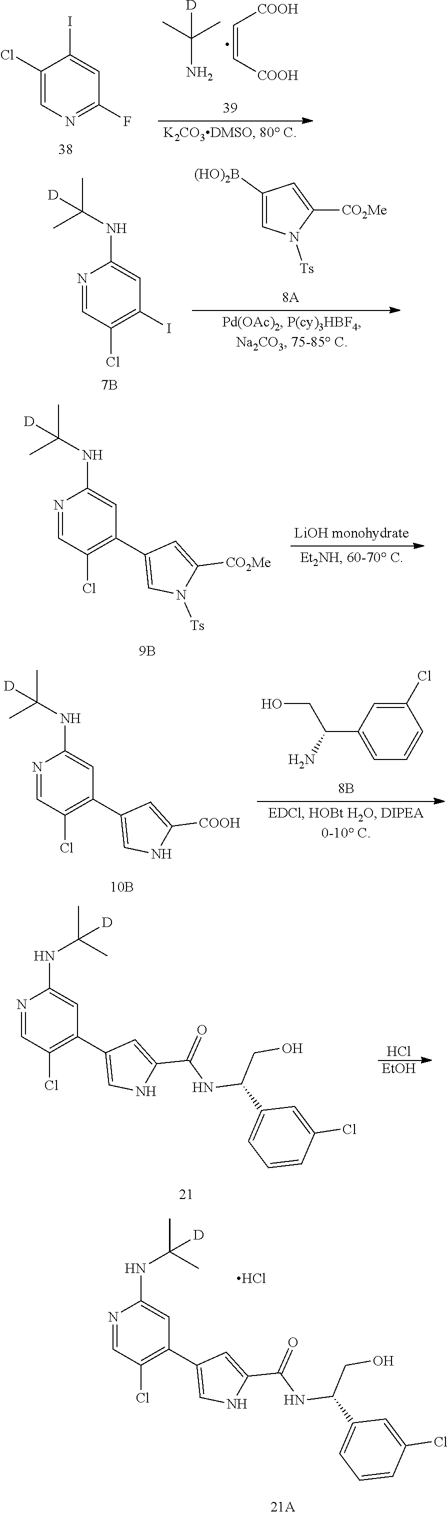

### Preparation of Formula 39:

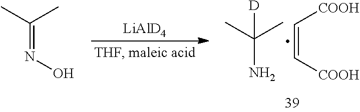

A solution of propan-2-one oxime (1.0 equiv.) in THF was charged to a suspension of LiAlD₄ (1.5 equiv.) in THF (16.7V) at 50 to 55° C. The mixture was stirred at 60-65° C. for 12 hr. The temperature was decreased to 0 to 10° C. and quenched by adding sodium sulfate (0.5 equiv.). The mixture was stirred for 1 hr and filtered. The filter cake was rinsed with THF (5V). The filtrate was transferred to a flask and maleic acid (1.0 equiv.) was added at 15 to 30° C. The mixture was stirred at 20 to 25° C. for 2 hr, and stirred for another 2 hr at a temperature of 0 to 10° C. The slurry was filtered and the filter cake was washed with THF (1V). The solids were dried under nitrogen to afford 1.95 kg of product (Formula 39) as an off-white solid. Yield: 39.0%, Chemical purity: 98.2%; Isotopic purity: 99%; ¹H-NMR (400 MHz, D₂O): δ 6.22 (s, 21H), 1.18 (s, 611).

### Preparation of Formula 7B:

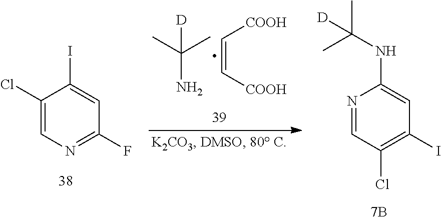

Formula 39 (3.0 equiv.) and K₂CO₃ (4.5 equiv.) were charged to a suspension of Formula 38 (1.0 equiv.) in DMSO (20V) at 15 to 30° C. The temperature of the mixture was increased to 75 to 85° C. and stirred for 24 hr. The temperature of the mixture was decreased to 15-25° C. and water (36V) was added. The product was extracted with EtOAc (6V×3) and the organic phases were combined. The combined organic phases were washed with water (10V×2) and aqueous NaCl (10V×1). The solution was concentrated under reduced pressure to 2V and n-heptane (5V) was added to the mixture. The heptane mixture was concentrated under reduced pressure to 2V and the process was repeated twice. (N-heptane (1V) was added to the concentrated residue and stirred at 15 to 25° C. for 4 hr. The mixture was filtered and the filter cake was dried at 20 to 30° C. under nitrogen for 12 hr to give 640 g of product (Formula 7B) as an off-white solid. Yield: 69.0%, HPLC purity: 98.9% (215 nm); HPLC assay purity: 97.3% (215 nm); Isotopic purity: 98%; ¹H-NMR (400 MHz, CDCl₃): δ 8.01 (s, 1H), 6.88 (s, 1H), 1.22 (s, 6H).

### Preparation of Formula 9B:

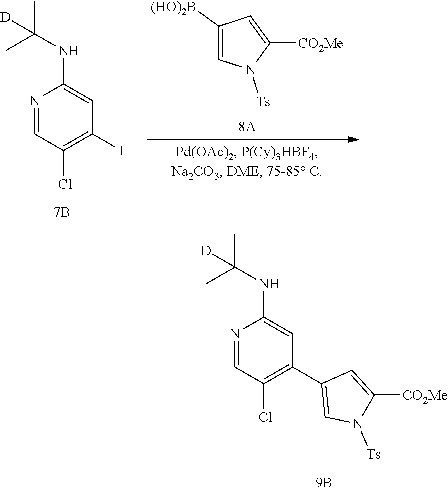 [SMILES: [2H]C(C)(C)Nc1cc(I)c(Cl)cn1 ; COC(=O)c1cc(B(O)O)cn1S(=O)(=O)c1ccc(C)cc1 ; [2H]C(C)(C)Nc1cc(-c2cc(C(=O)OC)n(S(=O)(=O)c3ccc(C)cc3)c2)c(Cl)cn1]

Purified water (1V) and anhydrous sodium carbonate (1.5 equiv.) were added to a solution of Formula 7B (1.0 equiv.) and Formula 8A (1.1 equiv.) in glycol dimethyl ether (8V at 20 to 30° C. The mixture was degassed with nitrogen for 30 min. Pd(OAc)₂ (0.02 equiv.) and P(cy)₃HBF₄, (0.05 equiv.) were added to the mixture under the protection of nitrogen. After the addition, the mixture was evacuated and filled with nitrogen. The process was repeated 10 times. The temperature of the mixture was increased to 75 to 85° C. and stirred for 4 hr. The temperature of the reaction mixture was decreased to 25-35° C. and filtered. The filter cake was rinsed with THF (1.3V×2). The filtrates were combined and concentrated under reduced pressure to 1V. MeOH (1.3 vol.) was added to the mixture at 15 to 25° C. and stirred for 2 hr at 15 to 25° C. The slurry filtered and the filter cake was rinsed with methanol (1V×2). The solids were re-slurried in a mixture of EtOAc:Hexane (1:6.5, 5.6V) at 15 to 25° C. for 2 hr. The slurry was filtered and the filter cake was rinsed with hexane (1V) and dried under nitrogen for 8 hr to give 351 g of product (Formula 9B) as a light yellow solid. Yield: 85.0%, HPLC purity: 96.5% (215 nm); HPLC assay purity: 95.1% (215 nm); Isotopic purity: 98%; ¹H-NMR (400 MHz, CDCl₃): δ 8.15 (s, 1H), 8.10 (s, 1H), 7.93-7.95 (m, 2H), 7.34-7.36 (m, 3H), 6.40 (s, 1H), 3.77 (s, 3H), 2.44 (s, 3H), 1.24 (s, 6H).

### Preparation of Formula 10B:

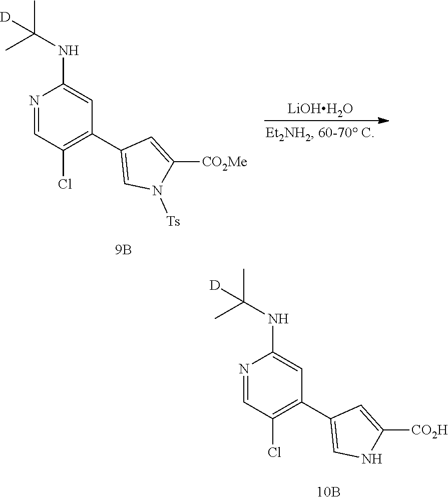 [SMILES: [2H]C(C)(C)Nc1cc(-c2cc(C(=O)OC)n(S(=O)(=O)c3ccc(C)cc3)c2)c(Cl)cn1 ; "LiOH·H2O" ; [2H]C(C)(C)Nc1cc(-c2c[nH]c(C(=O)O)c2)c(Cl)cn1]

An aqueous solution of LiOH (5.0 equiv., 7 wt. %) was added to a solution of Formula 9B (1.0 equiv.) in THF (5V) at 15 to 30° C. Then Et₂NH (2.0 equiv.) was added to the mixture and the mixture was stirred at 60 to 70° C. for 30 hr. The temperature of the mixture was reduced to 20 to 25° C. MTBE (5V) was added to the mixture at 15 to 25° C. and stirred for 30 min. The phases were separated and the organic phase was discarded. The pH of the aqueous phase was adjusted between 1 and 2 with 6N HCl. At 15 to 25° C. a solution of sodium carbonate in water was added to the mixture to adjust the pH between 9 and 10 to dissolve the solids. The pH of the solution was adjusted to 6.2 with 6N HCl. The mixture was filtered and the filter cake was re-slurried in water (10V) and stirred for 2 hr. The slurry was filtered and dried at 55 to 65° C. for 40 hr. The solids were slurried with MTBE (6V) at 20 to 25° C. for 2 hr and filtered. The filter cake was dried to give 138 g of product (Formula 10B) as an off-white solid. Yield: 65.0%, HPLC purity: 98.3% (215 nm); HPLC assay purity: 89.4% (215 nm); Isotopic purity: 99%; ¹H-NMR (400 MHz, DMSO-d₆): δ 7.97 (s, 1H), 7.49 (s, 1H), 7.13 (s, 1H), 6.62 (s, 1H), 1.13 (s, 6H).

### Preparation of Formula 21

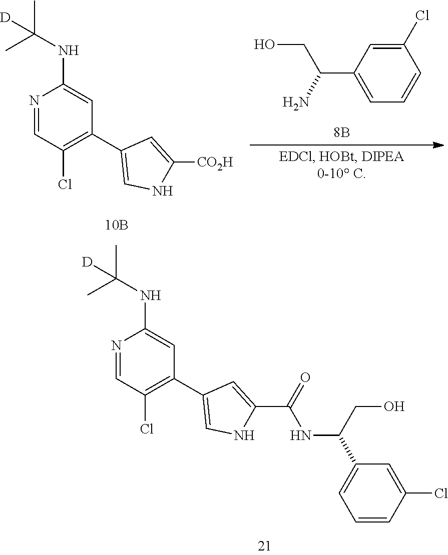 [SMILES: [2H]C(C)(C)Nc1cc(-c2c[nH]c(C(=O)O)c2)c(Cl)cn1 ; N[C@H](CO)c1cccc(Cl)c1 ; [2H]C(C)(C)Nc1cc(-c2c[nH]c(C(=O)N[C@H](CO)c3cccc(Cl)c3)c2)c(Cl)cn1]

HOBt (1.2 equiv.) and EDCI (1.1 equiv.) were added to a solution of Formula 10B (1.0 equiv.) in DMF (10V) at −10 to −20° C. The temperature of the mixture was decreased to −20 to −30° C. Formula 8B (1.05 equiv.) and DIPEA (1.2 equiv.) were then added. The mixture was stirred at 0 to 10° C. for 8 hr. The reaction mixture was diluted with ethyl acetate (9V) and water (4V). The phases were separated, and the aqueous phase was extracted with ethyl acetate (9V×3). The organic phases were combined and washed sequentially with water (4V×2). 5% HOAc (7V×2), aqueous Na₂CO₃ (7V×2, 17% by wt.) and brine (TOV×2). Silica gel (1 g/g) was added to the organic phase and stirred for 4 hr at 15 to 30° C., then filtered. The filter cake was rinsed with ethyl acetate (4.5V). The combined filtrate was concentrated under reduced pressure to 2V. MTBE (2V) was added to the mixture. The mixture was cooled to 0° C. to 10° C. and stirred for 1 hr. The mixture was filtered and the solids were dried to give 181 g of product (Formula 21) as a yellow solid. Yield: 69.0%, HPLC purity: 99.6% (215 nm); HPLC chiral purity (ee): 100.0% (254 nm); HPLC assay purity: 99.0% (215 nm); Isotopic purity: 99%; ¹H-NMR (400 MHz, DMSO-d₆): δ 7.98 (s, 1H), 7.45 (s, 1H), 7.38 (m, 4H), 7.31 (m, 1H), 6.61 (s, 1H), 5.08 (q, 1H), 3.69 (m, 2H), 1.13 (s, 6H).

### Preparation of Formula 21A

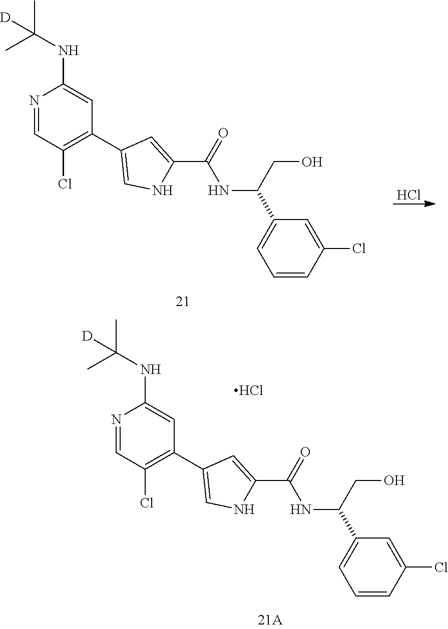 [SMILES: [2H]C(C)(C)Nc1cc(-c2c[nH]c(C(=O)N[C@H](CO)c3cccc(Cl)c3)c2)c(Cl)cn1 ; Cl ; [2H]C(C)(C)Nc1cc(-c2c[nH]c(C(=O)N[C@H](CO)c3cccc(Cl)c3)c2)c(Cl)cn1 ; Cl]

At 15 to 25° C., filtered anhydrous ethanol (145 g, 0.26V), filtered methanol (8.1 g, 0.02V), and filtered isopropanol (8.1 g, 0.02V) were charged into a flask and stirred for 20 to 30 min. Then hydrogen chloride gas was bubbled into the mixture under stirring at 10 to 25° C. After 2 hr, the mixture was sampled and analyzed every 2 to 4 hr until the content of hydrogen chloride reached ≥35% by weight. While maintaining the temperature at 15 to 25° C., filtered anhydrous ethanol (4292 g, 9V), filtered methanol (232 g, 0.5V), and filtered isopropanol (232 g, 0.5V) were charged into a separate 20 L reactor through an in-line fluid filter and stirred for 20 to 30 min. Then Formula 21 (580 g, 1.0 equiv.) was added. The temperature of the mixture was increased to 70 and 75° C. at the rate of 15 to 25° C./hr and stirred until the solids were completely dissolved. At 70 to 75° C., seed crystals (0.58 g, 0.1 wt. %) were added to the mixture, and then the anhydrous HCl solution (141 g) was added. The mixture was maintained at 70 to 75° C. for 1 to 2 hr with stirring. The temperature of the mixture was decreased to 15 to 25° C. at the rate of 5 to 15° C./hr and then maintained at 15 to 25° C. for 4 to 6 hr under stirring. The mixture was filtered and the filter cake was rinsed for 20 to 30 min with filtered MTBE. The filter cake was dried under nitrogen at 40 to 50° C. for 24 hr to afford 580 g of product (Formula 21A) as a white solid. Yield: 92.0%, HPLC purity: 99.9% (215 nm); HPLC chiral purity (ee): 100.0% (254 nm); HPLC assay purity: 99% (215 nm); Chloride content: 7.6%; Isotopic purity: 99%; ¹H-NMR (400 MHz, DMSO-d₆): δ 12.32 (s, 1H), 8.78 (d, 1H), 8.50 (s, 1H), 8.11 (s, 1H), 7.64 (s, 1H), 7.57 (s, 1H), 7.46 (s, 1H), 7.37 (m, 2H), 7.31 (m, 1H), 7.11 (s, 1H), 5.05 (q, 1H), 3.70 (m, 2H), 1.22 (s, 6H).

### Example 4

### Characterization Data of Deturated Analogs of Ulixertinib

Mechanism of Action (MOA) Cell Assay—A375 Cell Proliferation, pERK/ERK and pRSK/RSK Analysis:

### Results of A375 Proliferation Assay:

TABLE 2

| Compound | IC₅₀ (μM) |
|---|---|
| Ulixertinib | 0.25 |
| Formula 18 (D7) | 0.30 |
| Formula 21 (D1) | 0.31 |

Briefly, A375 cells were plated at 10,000 cells/well in three 96 well plates. Plates were incubated overnight at 37° C. and allowed to recover. Cells were washed with 150 μL of DPBS and 90 μL of fresh media was added to each well. The cells were then treated with a dose response of the selected compounds. Compounds were diluted from stock concentrations (10 mM in DMSO). Serial dilutions were made in DMSO, yielding 8 total different concentrations per compound. At time of compound addition, the control plate was analyzed using Cell TiterGlo to set baseline for proliferation. Compound treated cells were allowed to grow at 37° C./5% CO₂ for 72 hours then analyzed using Cell TiterGlo according to kit protocol.

A375 pERK/ERK Dose Response Study:

TABLE 3

| Compound | IC₅₀ (μM) |
|---|---|
| Ulixertinib | 0.058 |
| Formula 18 (D7) | 0.070 |
| Formula 21 (D1) | 0.064 |

Briefly, A375 cells were plated at 30,000 cells/well in 2×96 well plates, allowed to recover overnight, washed and fed fresh media the next day then treated with a dose response of the selected compounds. Following the determined pretreatment time with the compounds at 37° C./5% CO2, the media was aspirated, and cells lysed with cold, complete MESO Scale Discovery (MSD) lysis buffer. pERK and total ERK1/2 levels were measured using MESO Scale Discover Phospho(Thr202/Tyr204; Thr185/Tyr187)/Total ERK1/2 kits. The protocol and all reagents were provided in the kit. All wells were blocked with 150 μl Blocking Solution for one hour. 25 ml of cell lysate plus 25 ml of lysis buffer was added per well and incubated overnight at 4° C. Following the overnight incubation, the wells were washed three times with Tris Wash buffer, then 25 ml of 1× Detection Antibody was added per well and incubated at room temperature for one hour with vigorous shaking. All wells were then washed three times with Tris Wash Buffer before 150 μl of 1× Read Buffer was added per well and the plates read using the MSD Sector Imager. Data were calculated as a ratio of phospho-ERK1/2 to total ERK1/2 to allow for normalization of the samples.

A375 pRSK/RSK Dose Response Study

TABLE 4

| Compound | IC₅₀ (μM) |
|---|---|
| Ulixertinib | 0.037 |
| Formula 18 (D7) | 0.040 |
| Formula 21 (D1) | 0.037 |

Briefly, A375 cells were plated at 30,000 cells/well in 2×96 well plates, allowed to recover overnight, washed and fed fresh media the next day then treated with a dose response of the selected compounds. Following the determined pretreatment with the compounds at 37° C./5% CO2 the media was aspirated, and cells lysed with cold, complete MSD lysis buffer. pRSK and total RSK were analyzed using CST/PathScan pRSK and total RSK ELISAS according to the respective kit protocols. All samples were analyzed at 90% of remaining sample in pRSK and 10% of remaining sample in total RSK ELISA, which was performed following kit protocols.

### Half-Life Determination of Ulixertinib, Formula 18, Formula 19, and Formula 21 in Human Liver Microsomes (HLM):

TABLE 5
Half-life, t½ (min), Values Measured in Human Liver Microsomes

| | | | | | | | Mean ± |
|---|---|---|---|---|---|---|---|
| | | | | | | | SD (% |
| | | | | | | | change |
| | | | | | | | relative to |
| Compound ID | Rep 1 | Rep 2 | Rep 3 | Rep 4 | Rep 5 | Rep 6 | ulixertinib) |
| Ulixertinib | 49.2 | 51.2 | 50.3 | 46.2 | 48.5 | 48.1 | 48.9 ± 1.8 |
| Formula 21 | 60.9 | 75.8 | 64.4 | 61.5 | 59.5 | 60.8 | 63.8 ± 6.1 |
| | | | | | | | (+30%) |
| Formula 19 | 43.4 | 45.2 | 46.1 | 44.9 | 47.8 | 44.9 | 45.4 ± 1.5 |
| | | | | | | | (−7%) |
| Formula 18 | 59.2 | 58.7 | 60.6 | 55.8 | 57.5 | 62.0 | 59.0 ± 2.2 |
| | | | | | | | (+21%) |

Human microsomes from a pool of 10 donors was obtained from a commercial source. Microsomes prepared in potassium phosphate buffer were spiked with ulixertinib, Formula 18, Formula 19, and Formula 21 individually in replicates of six at a final concentration of 1 μM. NADPH was used to initiate the experiment; a control of minus NADPH (minus cofactor) was also run in parallel to the human liver microsome stability. After spiking with test article, microsome replicates were placed in a 37° C. incubator with the following time course: 0, 5, 10, 15, 20, 30, 45 and 60 minutes. Samples were extracted with a protein precipitation quench consisting of ice cold acetonitrile containing internal standard (tolbutamide). Samples were analyzed for ulixertinib, Formula 18, Formula 19, and Formula 21. The half-lives of ulixertinib, Formula 18, Formula 19, and Formula 21 were characterized by the natural log of the slope of the line from the % remaining vs time to determine the elimination rate (ke) and using the formula described below.

$${t}_{\frac{1}{2}} = \frac{Ln ( 0. 5 )}{ke} = minutes$$

ke (elimination rate): the slope of ln(percent remaining) vs time (in minutes)

The formation of the N-desalkyl, i-Propyl Hydroxyl, Aldehyde, pyridine N-oxide and Carboxylic Acid metabolites were monitored simultaneously with each parent test article. The percent metabolite formation values for the N-desalkyl (M2), i-Propyl Hydroxyl (M3), N-oxide (M6) and carboxylic acid (M7) metabolites were calculated based off the peak area of metabolite formed and test article peak area for the same time point.

$$\% formation = [ ( P {A}_{metabolite} ) / ( {PA}_{p 0} ) ] \times 100$$

PA_{metabolite}: peak area ratio of metabolite at a given time point
PAₚ₀: peak area ratio of test article at time zero

Table 5 shows that Formula 21 has a 30% longer half-life in the assay than ulixertinib, and that Formula 18 has a 21% longer half-life than ulixertinib, whereas Formula 19 has a 7% shorter half-life compared to ulixertinib.

Mean Percent Observed of Metabolites Formed Following Incubation with Human Liver Microsomes:

TABLE 6
Percent Observed for Metabolites Following Incubation
in Human Liver Microsomes (Mean ± SD at 60 min)

| Compound | Formula 34 | Formula 35 | Formula 37 | Formula 36 |
|---|---|---|---|---|
| ID | M2 | M3 | M6 | M7 |
| Ulixertinib | 16.5 ± 3.1 | 3.83 ± 0.69 | 11.2 ± 1.4 | 0.189 ± 0.054 |
| Formula 21 | 7.78 ± 0.60 | 2.88 ± 0.52 | 10.3 ± 0.2 | 0.239 ± 0.041 |
| Formula 19 | 14.0 ± 2.4 | 0.00 ± 0.00 | 10.4 ± 1.1 | 0.00 ± 0.00 |
| Formula 18 | 5.11 ± 0.47 | 0.00 ± 0.00 | 17.8 ± 1.8 | 0.00 ± 0.00 |

Table 6 shows that Formula 21 (D1) decreases formation of M2 by 53%, decreases formation of M3 by 25%, decreases formation of M6 by 8%, and increases formation of M7 by 26% relative to ulixertinib in the assay. Formula 19 (D6) decreases formation of M2 by 15%, decreases formation of M3 by 100%, decreases formation of M6 by 7%, and decreases formation of M7 by 100% relative to ulixertinib in the assay. Formula 18 decreases formation of M2 by 69%, decreases formation of M3 by 100%, increases formation of M6 by 59%, and decreases formation of M7 by 100% relative to ulixertinib in the assay.

### Evaluation of Pharmacokinetics and Metabolite Formation in Rats for Example Compounds Formulas 18, 19, and 21:

Ulixertinib, Formulas 18A, 19A, and 21A were dosed twice daily (12 hours apart, ±30 minutes) for five consecutive days (10 doses/animal) to rats via oral gavage (PO). Each compound was administered at a dose of 12.5 mg/kg to 6 rats (3 male/3 female, N=6 rats/compound). Each compound was formulated in 1% (w/v) carboxymethylcellulose (CMC) in water at a concentration of 1.25 mg/mL. Following the second (p.m.) dose on Day 5, blood samples were collected from each rat at approximately 0.5, 1, 2, 4, 6, 8, and 12 hours post-dose. Blood samples were centrifuged to obtain plasma. Plasma samples were analyzed for concentrations of the dosed compounds and selected metabolites using an established LC-MS/MS method. The lower limit of quantitation of each compound was 1.00 ng/mL. The rat half-life values (determined by non-compartmental analysis using WinNonlin software) for each compound are shown in Table 7, and the areas under curve from time 0 to hour 12 (AUC₀₋₁₂), calculated using the linear trapezoidal rule, for each dosed compound and selected metabolites are presented in Table 8.

TABLE 7
Half-life, t^{1/2} (hr), Values Measured in Rat Plasma

| | | t_{1/2} |
|---|---|---|
| Compound Dosed | | (hr) |
| Ulixertinib | Mean | 3.67 |
| | SD | 1.44 |
| | CV % | 39.3 |
| Formula 21A - D1 | Mean | 3.81 |
| | | (+4%^{a}) |
| | SD | 1.27 |
| | CV % | 33.3 |
| Formula 19A - D6 | Mean | 4.00 |
| | | (+9%^{a}) |
| | SD | 1.18 |
| | CV % | 29.4 |
| Formula 18A - D7 | Mean | 4.18 |
| | | (+14%^{a}) |
| | SD | 0.813 |
| | CV % | 19.4 |

^{a}Percent change relative to Ulixertinib

Table 7 shows that Formula 21A (D1) has a 4% longer mean half-life than ulixertinib, Formula 19A (D6) has a 9% longer mean half-life than ulixertinib, and that Formula 18A (D7) has a 14% longer mean half-life than ulixertinib.

TABLE 8
AUC₀₋₁₂ (μg*hr/mL) for Parent
and Related Metabolites in Rat Plasma

| Compound | | | | | |
|---|---|---|---|---|---|
| Dosed | | Parent | M3 | M2 | M6 |
| Ulixertinib | Mean | 173.0 | 0.725 | 17.8 | 0.203 |
| | SD | 43.1 | 0.147 | 7.38 | 0.124 |
| | CV % | 24.8 | 20.2 | 41.4 | 61.1 |
| Formula 21A - | Mean | 193.0 | 0.784 | 14.7 | 0.217 |
| D1 | | (+12%^{a}) | (+8%^{a}) | (−17%^{a}) | (+7%^{a}) |
| | SD | 64.5 | 0.209 | 6.15 | 0.127 |
| | CV % | 33.4 | 26.7 | 42.0 | 58.5 |
| Formula 19A - | Mean | 199.0 | 0.113 | 14.7 | 0.132 |
| D6 | | (+15%^{a}) | (−84%^{a}) | (−17%^{a}) | (−35%) |
| | SD | 80.5 | 0.0433 | 7.16 | 0.0633 |
| | CV % | 40.4 | 38.3 | 48.6 | 47.8 |
| Formula 18A - | Mean | 228.0 | 0.179 | 17.1 | 0.254 |
| D7 | | (+32%^{a}) | (−75%^{a}) | (−4%^{a}) | (+25%^{a}) |
| | SD | 87.6 | 0.0802 | 8.71 | 0.104 |
| | CV % | 38.5 | 44.8 | 51.0 | 41.0 |

^{a}Percent change relative to Ulixertinib

### Evaluation of Inhibition and Induction of Cytochrome P450 Metabolic Enzymes:

### In-Vitro CYP Inhibition Study:

This study was designed to evaluate the ability of four test articles to inhibit the major CYP enzymes in human liver microsomes (namely, CYP1A2, CYP2B6, CYP2C8, CYP2C9, CYP2C19, CYP2D6 and CYP3A4/5) in a direct and time-dependent manner. Incubations were conducted using a cocktail of seven prototypical CYP substrates to ascertain the potential of each test article to inhibit the metabolism of concomitantly administered drugs. A mixed-gender pool of 200 human liver microsomal samples was used for this study. Incubations were conducted at approximately 37° C. in 200 μL incubation mixtures (pH 7.4) containing water, potassium phosphate buffer (50 mM), MgCl2 (3 mM), EDTA (1 mM), an NADPH regenerating system (NADP [1 mM], glucose 6 phosphate [5 mM], glucose 6 phosphate dehydrogenase [1 Unit/mL]), and probe substrate (i.e., a seven substrate cocktail) at the final concentrations indicated in Table 9. Reactions were initiated by the addition of NADPH-regenerating system and were automatically terminated at approximately 5 minutes by the addition of internal standard mixed in stop reagent, acetonitrile. The samples were centrifuged at 920×g for 10 minutes at 10° C. The supernatant fractions were analyzed by LC-MS/MS. To examine its ability to act as a time-dependent inhibitor of CYP enzymes, each test article (at the same concentrations used to evaluate direct inhibition, (i.e. 0, 0.02, 0.06, 0.2, 0.6, 2, 6, 20 μM) was pre-incubated at 37±2° C. with NADPH-fortified human liver microsomes for approximately 30 minutes. The pre-incubations were initiated by the addition of NADPH-regenerating system. After 30 min, probe substrate incubations were initiated by the addition of probe substrate cocktail. Incubations were terminated and samples were analyzed by LC-MS/MS. When inhibition was observed, the data were processed with a LIMS for the determination of IC₅₀ values by utilizing the Levenberg-Marquardt algorithm to perform non-linear regression fitting of the data to the following 4-parameter sigmoidal-logistic IC₅₀ equation:

$$Y = Min + \frac{( Max - Min )}{( 1 + {( C onc / {IC}_{50} )}^{slope} )}$$

As percent of control values are utilized, the minimum value (Min) is set to zero and the maximum value (Max) is set to 100 (or other values, as appropriate). The LIMS calculates an IC₅₀ value only when it lies within the concentration range of the inhibitor studied. Therefore, when an IC₅₀ value falls outside the concentration range studied, the IC₅₀ values are reported to be greater than the highest concentration of the test article evaluated.

TABLE 9
IC₅₀ (μM) Values for Cytochrome P450 Enzymes Measured in Human Liver Microsomes

| | CYP1A2 | | CYP2B6 | | CYP2C19 | | CYP2C8 | | CYP2C9 | | CYP2D6 | | CYP3A4/5 | |
|---|---|---|---|---|---|---|---|---|---|---|---|---|---|---|
| | Substrate Conc. (μM) | | | | | | | | | | | | | |
| | Phenacetin | | Bupropion | | S-Mephenytoin | | Paclitaxel | | Diclofenac | | Dextramethorphan | | Midazolam | |
| | (90) | | (90) | | (60) | | (5) | | (12) | | (10) | | (3) | |
| | Preincubation time (min) | | | | | | | | | | | | | |
| | 0 | 30 | 0 | 30 | 0 | 30 | 0 | 30 | 0 | 30 | 0 | 30 | 0 | 30 |
| Ulixertinib | >20 | 13 | 11 | 16 | 8.5 | 9.2 | 8.5 | 9.2 | 3.1 | 3.4 | >20 | 18 | >20 | 14 |
| Formula 21A- | >20 | >20 | 17 | 19 | 19 | 19 | 19 | 19 | 3.4 | 4.4 | >20 | >20 | >20 | 17 |
| D1 | | | | | | | | | | | | | | |
| Formula 19- | >20 | 19 | 14 | 16 | 10 | 12 | 10 | 12 | 3.1 | 4.3 | 20 | >20 | >20 | 13 |
| D6 | | | | | | | | | | | | | | |
| Formula 18- | >20 | 17 | 12 | 16 | 9.6 | 11 | 9.6 | 11 | 3.5 | 3.9 | >20 | 19 | >20 | 14 |
| D7 | | | | | | | | | | | | | | |

### In-Vitro CYP Induction Study:

This study was designed to evaluate the ability of four test articles (BVD-523ulixertinib, Formula 18, Formula 21A, and Formula 19) to induce the mRNA expression of the major CYP enzymes in cultured human hepatocytes (namely CYP1A2, CYP2B6 and CYP3A4). A single preparation of characterized cryopreserved human hepatocytes isolated from non-transplantable human livers was treated in this study. Triplicate wells for each of the cultures were treated in 48-well plates with supplemented MCM (37±2° C.) containing 0.1% v/v DMSO (solvent control), one of three concentrations of each test article (1, 10 and 20 μM) or positive control CYP enzyme inducers omeprazole (50 μM), phenobarbital (1000 μM) or rifampin (20 μM). The cultures were incubated in a humidified culture chamber (37±2° C. at 95% relative humidity, 95/5% air/CO₂). Approximately 24 h after treatment, hepatocytes were lysed in Buffer RLT reagent containing β-mercaptoethanol (100:1). The medium was aspirated, and Buffer RLT was added to each well. The cell lysates were prepared by repeated pipetting and shaking. Total RNA was isolated using the RNeasy Mini Kit. RNA quality and concentration were determined by measuring absorbance at 260 and 280 nm on a plate reader. Single-stranded cDNA was prepared from RNA with the RT Master Mix. The RT Master Mix comprises 10×RT buffer, 25× deoxyNTPs, 10× Random primers, RNase Inhibitor (20 U/μL), MultiScribe reverse transcriptase (50 U/μL) and RNase-free water. The RT Master Mix was added to each RNA sample to complete the components of the reaction. No template controls (NTCs) were included in the analysis. For the NTC reactions, RNase-free water was added in place of the RNA sample. The prepared cDNA samples were stored at −20±5° C. following analysis by qRT-PCR. Each PCR was performed in quadruplicate. A Primer Mix was prepared for each Gene Expression assay. A typical Primer Mix contained TaqMan Fast Advanced Master Mix (1×), Gene Expression Assay (1×, 900 nM forward and reverse primers) and RNase-free water. The Reaction Mix was prepared by adding the Primer Mix to cDNA. A percentage of samples (no less than 10%) included NAC (no amplification control) samples. NAC samples are RNA samples that are not reverse transcribed and are used to show that mRNA, not genomic DNA, is the source of PCR's fluorescent signal. Reactions were analyzed on a PCR sequence detection system. The relative quantity of the target cDNA compared with that of the control cDNA (GAPDH) was determined by the ΔΔCₜ method. Relative quantitation measures the change in mRNA expression in a test sample relative to that in a control sample (e.g., DMSO). This method assumes that the efficiency of the target amplification and the efficiency of the endogenous control amplification are approximately equal.

Treatment with up to 20 μM of ulixertinib, Formula 18, Formula 21A, and Formula 19 resulted in increases in CYP1A2 mRNA expression up to 6.63-, 7.56-, 8.51- and 12.1-fold change, respectively. Of note, maximal fold change was observed at 10 μM for ulixertinib, Formula 21A, and Formula 19, followed by a decline to 6.42-, 4.00- and 9.52-fold change, respectively. Treatment with up to 20 μM of ulixertinib, Formula 18, Formula 21A, and Formula 19 resulted in concentration-dependent increases in CYP2B6 mRNA expression up to 2.43, 2.54-, 2.22- and 2.44-fold change, respectively. Treatment with up to 20 μM of ulixertinib, Formula 18, Formula 21A, and Formula 19 resulted in concentration-dependent increases in CYP3A4 mRNA expression up to 8.26-, 7.06-, 6.86- and 11.7-fold change, respectively. Results are summarized in TABLE 10, below.

TABLE 10
Fold Change in mRNA Expression of Cytochrome P450
Enzymes in Cultured Human Hepatocytes

| | CYP1A2 | | | CYP2B6 | | | CYP3A4 | | |
|---|---|---|---|---|---|---|---|---|---|
| Treatment Group | 1 μM | 10 μM | 20 μM | 1 μM | 10 μM | 20 μM | 1 μM | 10 μM | 20 μM |
| Ulixertinib | 3.73 | 6.63 | 6.42 | 1.25 | 1.99 | 2.43 | 1.52 | 6.84 | 8.26 |
| Formula 21A-D1 | 2.22 | 8.51 | 4.00 | 1.08 | 2.04 | 2.22 | 1.39 | 5.29 | 6.86 |
| Formula 19-D6 | 3.57 | 12.1 | 9.52 | 1.47 | 2.10 | 2.44 | 2.06 | 9.42 | 11.7 |
| Formula 18-D7 | 3.02 | 5.94 | 7.56 | 1.36 | 1.90 | 2.54 | 1.83 | 5.39 | 7.06 |

### CITED DOCUMENTS

Kempf, D. et al. Antimicrobial Agents and Chemotherapy, 41(3), p. 654 (1997);
Wang, L. et al. Clinical Pharmacology and Therapeutics, 56(6 Pt. 1), p. 659 (1994);
Blake, M. et al. J. Pharm. Sci., 64, p. 367 (1975);
Foster, A. Adv. Drug Res., 14, p. 1 (1985);
Kushner, D. et al. Can. J. Physiol. Pharmacol., p. 79 (1999);
Fisher M. et al. Curt. Opin. Drug Discov. Devel., 9, p. 101 (2006);
Fukuto, J. et al. J. Med. Chem., 34(9), p. 2871 (1991);
Harbeson, L. and Tung. R. Medchem News, 2, p. 8 (2014);
Scott L. Harbeson and Roger D. Tung, Deuterium in Drug Discovery and Development, 46 annual report in medicinal chemistry, 403-417 (2011);
Miwa. G., Lu, A., Kinetic Isotope Effects and ‘Metabolic Switching’ in Cytochrome P450-Catalyzed Reactions, 7 Bioessays, 215-19 (1987);
Allan B. Foster, “Deuterium Isotope Effects in the Metabolism of Drugs and Xenobiotics: Implications for Drug Design”, Advances in Drug Research, (14), 1-40 (1985)
Shao et. al., “Derivatives of Tramadol for Increased Duration of Effect”, Bioorganic and Medicinal Chemistry Letters, (16), 691-94 (2006);
Bin Yu et al., “Pharmacokinetics and metabolism of ulixertinib in rat by liquid chromatography combined with electrospray ionization tandem mass spectrometry,” Separation Science, vol. 43, issue 7, pages 1275-1283 (2020);
“The Organic Chemistry of Drug Design and Drug Interaction” Richard Silverman, published by Academic Press (1992);
Remington's Pharmaceutical Sciences, Sixteenth Edition, E. W. Martin (Mack Publishing Co., Easton, Pa., 1980.

All documents cited in this application are hereby incorporated by reference as if recited in full herein.

Although illustrative embodiments of the present invention have been described herein, it should be understood that the invention is not limited to those described, and that various other changes or modification may be made by one of ordinary skill in the art without departing from the scope or spirit of the invention.

What is claimed is:

1. A compound of Formula 1: 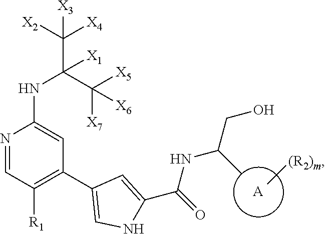
or a pharmaceutically acceptable salt, solvate, or prodrug thereof, wherein each of X₁, X₂, X₃, X₄, X₅, X₆, and X₇ is independently selected from the group consisting of hydrogen, deuterium, and C₁₋₄ aliphatic, and wherein:
R₁ is hydrogen, C₁₋₃ aliphatic, fluoro, or chloro;
A is an optionally substituted group selected from phenyl, a 5-6 membered monocyclic heteroaryl ring having 1-5 heteroatoms independently selected from nitrogen, oxygen, or sulfur, or a 5-6 membered saturated or partially unsaturated heterocyclic ring having 1-5 heteroatoms independently selected from nitrogen, oxygen, or sulfur;
R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; and
m is 0, 1, or 2.

2. A compound of Formula 2A: 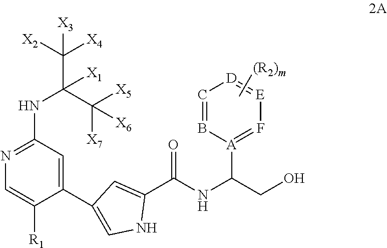
or a pharmaceutically acceptable salt, solvate, or prodrug thereof, wherein: each X₁, X₂, X₃, X₄, X₅, X₆, and X₇ are independently selected from hydrogen and deuterium or C₁₋₄ aliphatic; R² is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; m is 0, 1, or 2; and A, B, C, D, E, and F are independently selected from carbon or nitrogen.

3. A compound of Formula 2B: 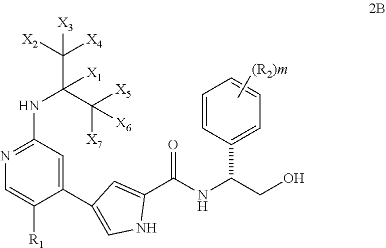
or a pharmaceutically acceptable salt, solvate, or prodrug thereof, wherein: each X₁, X₂, X₃, X₄, X₅, X₆, and X₇ are independently selected from hydrogen and deuterium or C₁₋₄ aliphatic; R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; and m is 0, 1, or 2.

4. A compound of Formula 3A: 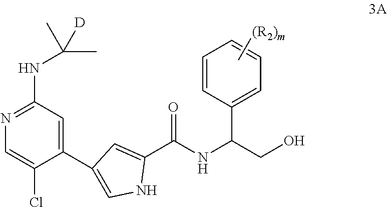
or a pharmaceutically acceptable salt, solvate, or prodrug thereof, wherein: R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; and m is 0, 1, or 2.

5. A compound of Formula 3B: 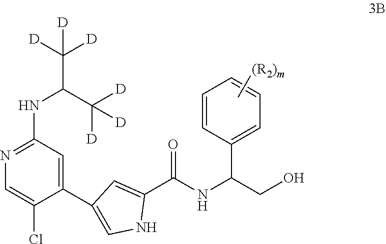
or a pharmaceutically acceptable salt, solvate, or prodrug thereof, wherein: R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; and m is 0, 1, or 2.

6. A compound of Formula 3C: 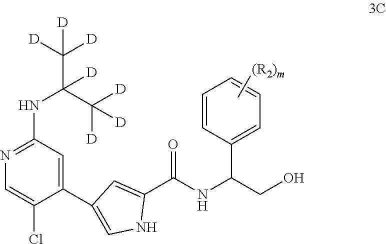
or a pharmaceutically acceptable salt, solvate, or prodrug thereof, wherein: R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂, wherein each R is independently hydrogen or C₁₋₄ aliphatic; and m is 0, 1, or 2.

7. A compound of Formula 4: 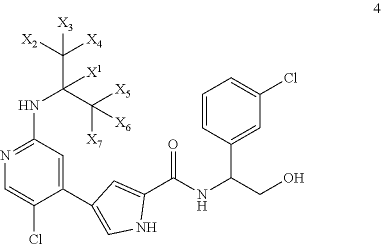
or a pharmaceutically acceptable salt, solvate, or prodrug thereof, wherein:
each X₁, X₂, X₃, X₄, X₅, X₆, and X₇ is independently selected from hydrogen and deuterium or C₁₋₄ aliphatic.

8. The compound of claim 1, which is selected from:
4-(5-chloro-2-((propan-2-yl-d7)amino)pyridin-4-yl)-N-(1-(3-chlorophenyl)-2-hydroxyethyl)-1H-pyrrole-2-carboxamide,
4-(5-chloro-2-((propan-2-yl-1,1,1,3,3,3-d6)amino)pyridin-4-yl)-N-(1-(3-chlorophenyl)-2-hydroxyethyl)-1H-pyrrole-2-carboxamide,
4-(5-chloro-2-((propan-2-yl-1,1,1,2,3-d5)amino)pyridin-4-yl)-N-(1-(3-chlorophenyl)-2-hydroxyethyl)-1H-pyrrole-2-carboxamide,
4-(5-chloro-2-((propan-2-yl-1,1,1,2-d4)amino)pyridin-4-yl)-N-(1-(3-chlorophenyl)-2-hydroxyethyl)-1H-pyrrole-2-carboxamide,
4-(5-chloro-2-((propan-2-yl-1,1,2-d3)amino)pyridin-4-yl)-N-(1-(3-chlorophenyl)-2-hydroxyethyl)-1H-pyrrole-2-carboxamide,
4-(5-chloro-2-((propan-2-yl-1,2-d2)amino)pyridin-4-yl)-N-(1-(3-chlorophenyl)-2-hydroxyethyl)-1H-pyrrole-2-carboxamide,
4-(5-chloro-2-((propan-2-yl-2-d1)amino)pyridin-4-yl)-N-(1-(3-chlorophenyl)-2-hydroxyethyl)-1H-pyrrole-2-carboxamide,
and pharmaceutically acceptable salts, solvates, and prodrugs thereof.

9. The compound of any one of claims 1-8, which is an enantiomerically pure 1S- or 1R-ulixertinib analog.

10. The compound of any one of claims 1-8, wherein any atom not designated as deuterium is present at its natural isotopic abundance.

11. The compound of any one of claims 1-8, wherein each of said positions having deuterium has deuterium enrichment of at least 1%.

12. A pharmaceutical composition comprising a compound according to any one of claims 1-8 and a pharmaceutically acceptable carrier, excipient or vehicle.

13. A method of treating a disease, disorder, or condition comprising the step of administering a therapeutically effective amount of a compound according to any one of claims 1-8 or the pharmaceutical composition of claim 12 to a subject in need thereof, wherein the disease, disorder, or condition comprises one or more of cancer, autoimmune disorders, neurodegenerative and neurological disorders, schizophrenia, bone-related disorders, liver disease, and cardiac disorders.

14. The method of claim 13, wherein after administering the therapeutically effective amount of the compound or pharmaceutical composition, there is a decreased metabolism of the compound per dosage unit thereof by at least one polymorphically-expressed cytochrome P₄₅₀ isoform as compared to the corresponding non-isotopically enriched compound.

15. The method of claim 14, wherein the cytochrome P₄₅₀ isoform is selected from the group consisting of CYP3A4, CYP3A5, CYP2C8, CYP2C9, CYP2D6, CYP2C19, CYP1A2, CYP2B6, and CYP2E1.

16. The method of claim 12, wherein the compound has decreased inhibition of at least one cytochrome P₄₅₀ per dosage unit thereof as compared to the non-isotopically enriched compound.

17. The method of claim 16, wherein said cytochrome P₄₅₀ is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3ASP1, CYPa5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

18. The compound of claim 1 or pharmaceutical composition of claim 12 for use as a medicament.

19. The compound of claim 1 or pharmaceutical composition of claim 12 for use in the manufacture of a medicament for the prevention or treatment of a disorder ameliorated by the inhibition of ERK protein kinase.

20. The compound of claims 1 to 8 or pharmaceutical composition of claim 12, wherein each position having deuterium has at least 1% incorporation of deuterium.

21. The compound of claim 1 to 3 or pharmaceutical composition of claim 12, wherein at least one of X₁, X₂, X₃, X₄, X₅, X₆, and X₇ is deuterium.

22. The compound of claim 1 to 3 or pharmaceutical composition of claim 12, wherein at least two of X₁, X₂, X₃, X₄, X₅, X₆, and X₇ is deuterium.

23. The compound of claim 1 to 3 or pharmaceutical composition of claim 12, wherein at least three of X₁, X₂, X₃, X₄, X₅, X₆, and X₇ is deuterium.

24. The compound of claim 1 to 3 or pharmaceutical composition of claim 12, wherein at least four of X₁, X₂, X₃, X₄, X₅, X₆, and X₇ is deuterium.

25. The compound of claim 1 to 3 or pharmaceutical composition of claim 12, wherein at least five of X₁, X₂, X₃, X₄, X₅, X₆, and X₇ is deuterium.

26. The compound of claim 1 to 3 or pharmaceutical composition of claim 12, wherein at least six of X₁, X₂, X₃, X₄, X₅, X₆, and X₇ is deuterium.

27. The compound of claim 1 to 3 or pharmaceutical composition of claim 12, wherein each of X₁, X₂, X₃, X₄, X₅, X₆, and X₇ is deuterium.

28. The compound of claim 1 to 3 or pharmaceutical composition of claim 12, wherein each of X₂, X₃, X₄, X₅, X₆, and X₇ is deuterium, and X¹ is hydrogen.

29. The compound of claim 1 to 3 or pharmaceutical composition of claim 12, wherein each of X₂, X₃, X₄, X₅, X₆, and X₇ is hydrogen, and X¹ is deuterium.

30. A method of inhibiting ERK1/2 in a cell comprising the step of contacting the cell with the compound of claim 1 to 8 or pharmaceutical composition of claim 12.

31. The compound of claim 1 to 8 or pharmaceutical composition of claim 12, wherein the compound or pharmaceutical composition is effective to decrease metabolism of the compound or pharmaceutical composition by at least one polymorphically-expressed cytochrome P₄₅₀ isoform as compared to the corresponding non-isotopically enriched compound.

32. The compound or pharmaceutical composition of claim 31, wherein the cytochrome P₄₅₀ isoform is selected from the group consisting of CYP1A1, CYP1A2, CYP1B1, CYP2A6, CYP2A13, CYP2B6, CYP2C8, CYP2C9, CYP2C18, CYP2C19, CYP2D6, CYP2E1, CYP2G1, CYP2J2, CYP2R1, CYP2S1, CYP3A4, CYP3A5, CYP3ASP1, CYPa5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

33. The compound or pharmaceutical composition of claim 32, wherein the cytochrome P₄₅₀ isoform is selected from the group consisting of CYP3A4, CYP3A5, CYP2C8, CYP2C9, CYP2D6, CYP2C19, CYP1A2, CYP2B6, and CYP2E1.

34. The compound of claim 1 to 8 or pharmaceutical composition of claim 12, which, when administered to a subject in need thereof, results in a decrease in metabolism of the compound or pharmaceutical composition compared to the corresponding non-deuterated form of the compound or pharmaceutical composition which is at least greater than about 5%, greater than about 10%, greater than about 20%, greater than about 30%, greater than about 35%, greater than about 40%, greater than about 45%, greater than about 50%, greater than about 55%, or greater than about 60% as compared to non-isotopically enriched compound.

35. A method of synthesizing a deuterated compound according to Formula 1: 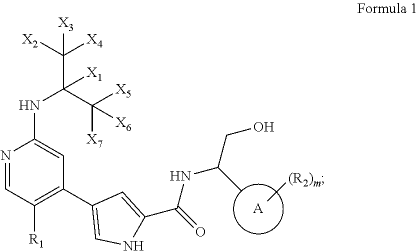
comprising the steps of:
(i) reacting a compound of Formula 5: 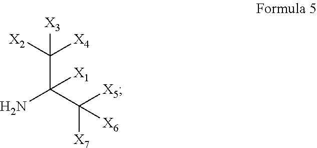
with a compound of Formula 6: 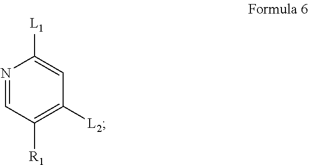
to produce the compound of Formula 7: 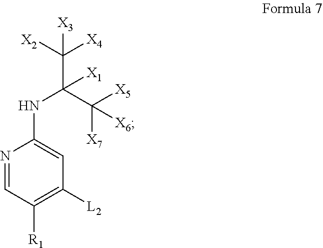
(ii) reacting the compound of Formula 7 with the compound of Formula 8: 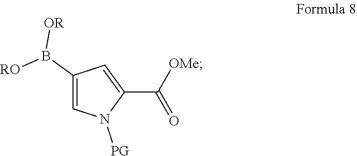
to produce the compound of Formula 9: 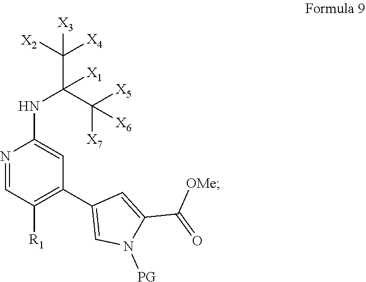
(iii) reacting the compound of Formula 9 with LiOH to produce the compound of Formula 10: 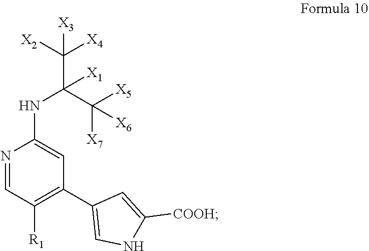
(iv) reacting the compound of Formula 10 with the compound of Formula 11: 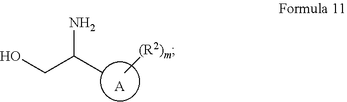
to produce the compound of Formula 1: 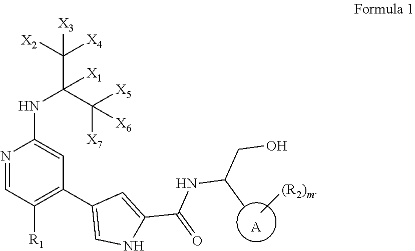
wherein each of X₁, X₂, X₃, X₄, X₅, X₆, and X₇ is independently selected from the group consisting of hydrogen, deuterium, and C₁₋₄ aliphatic, and wherein:
R₁ is hydrogen, C₁₋₃ aliphatic, fluoro, or chloro;
A is an optionally substituted group selected from phenyl, a 5-6 membered monocyclic heteroaryl ring having 1-5 heteroatoms independently selected from nitrogen, oxygen, or sulfur, or a 5-6 membered saturated or partially unsaturated heterocyclic ring having 1-5 heteroatoms independently selected from nitrogen, oxygen, or sulfur;
R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂; wherein each R is independently hydrogen or C₁₋₄ aliphatic;
m is 0, 1, or 2,
PG is a protecting group; and
L₁ and L₂ are independently selected leaving groups.

36. A method of synthesizing a deuterated compound according to Formula 1: 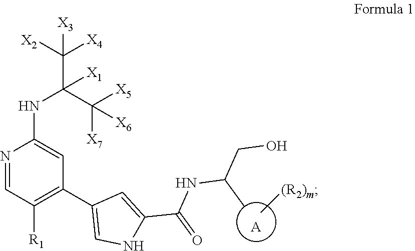
comprising the steps of:
(i) reacting a compound of Formula 12: 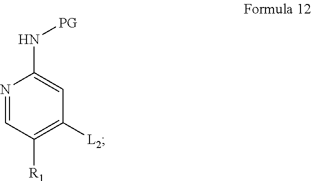
with a compound of Formula 8: 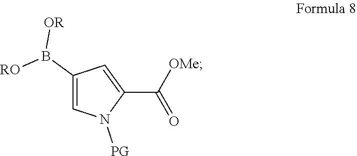
to produce a compound of Formula 13: 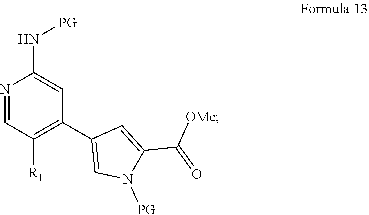
(ii) reacting the compound of Formula 13 with LiOH to produce the compound of Formula 14: 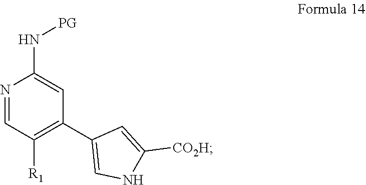
(iii) reacting the compound of Formula 14 with 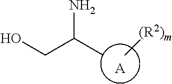
to produce the compound of Formula 15: 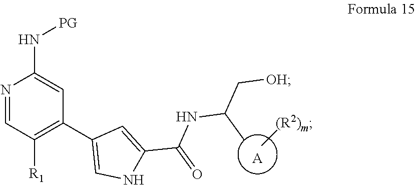
(iv) deprotecting the compound of Formula 15 to produce the compound of Formula 16: 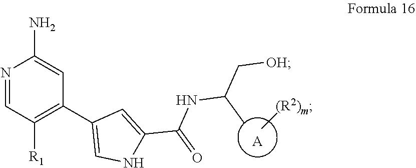
(v) reacting the compound of Formula 16 with the compound of Formula 17: 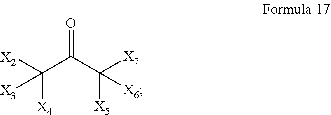
to produce the compound of Formula 1: 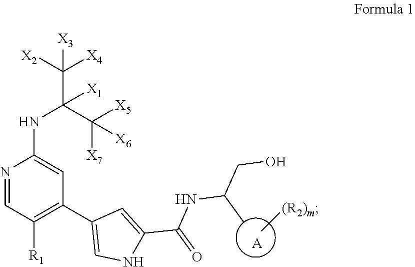
wherein each of X₁, X₂, X₃, X₄, X₅, X₆, and X₇ is independently selected from the group consisting of hydrogen, deuterium, and C₁₋₄ aliphatic, and wherein:
R₁ is hydrogen, C₁₋₃ aliphatic, fluoro, or chloro;
A is an optionally substituted group selected from phenyl, a 5-6 membered monocyclic heteroaryl ring having 1-5 heteroatoms independently selected from nitrogen, oxygen, or sulfur, or a 5-6 membered saturated or partially unsaturated heterocyclic ring having 1-5 heteroatoms independently selected from nitrogen, oxygen, or sulfur;
R₂ is independently —R, halogen, -haloalkyl, —OR, —SR, —CN, —NO₂, —SO₂R, —SOR, —C(O)R, —CO₂R, —C(O)N(R)₂, —NRC(O)R, —NRC(O)N(R)₂, —NRSO₂R, or N(R)₂; wherein each R is independently selected from hydrogen or C1′4 aliphatic;
m is 0, 1, or 2,
PG is a protecting group; and
L₂ is a leaving group.

37. A method of synthesizing a deuterated ulixertinib of Formula 18: 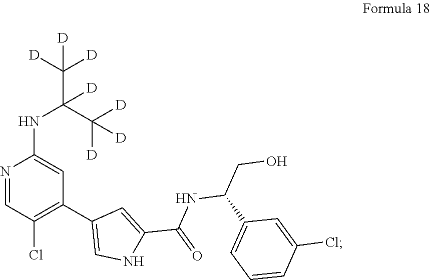 [SMILES: [2H]C([2H])([2H])C([2H])(Nc1cc(-c2c[nH]c(C(=O)N[C@H](CO)c3cccc(Cl)c3)c2)c(Cl)cn1)C([2H])([2H])[2H]]
or a pharmaceutically acceptable salt thereof, comprising the steps of:
(i) reacting a compound of Formula 7A: 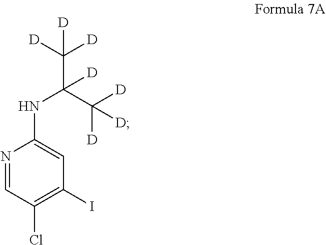
with a compound of Formula 8A: 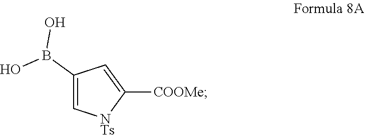 [SMILES: COC(=O)c1cc(B(O)O)cn1S(=O)(=O)c1ccc(C)cc1]
to provide the compound of Formula 9A: 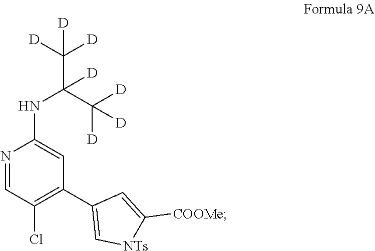 [SMILES: [2H]C([2H])([2H])C([2H])(Nc1cc(-c2cc(C(=O)OC)n(S(=O)(=O)c3ccc(C)cc3)c2)c(Cl)cn1)C([2H])([2H])[2H]]
(ii) reacting the compound of Formula 9A with LiOH to produce the compound of Formula 10A: 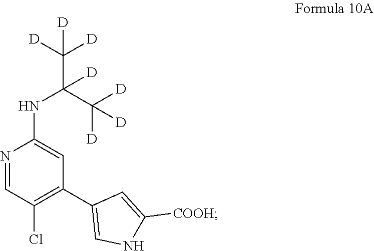 [SMILES: [2H]C([2H])([2H])C([2H])(Nc1cc(-c2c[nH]c(C(=O)O)c2)c(Cl)cn1)C([2H])([2H])[2H]]
and
(iii) reacting the compound of Formula 10A with the compound of Formula 8B: 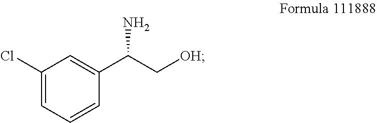 [SMILES: N[C@H](CO)c1cccc(Cl)c1]
to produce the compound of Formula 18:

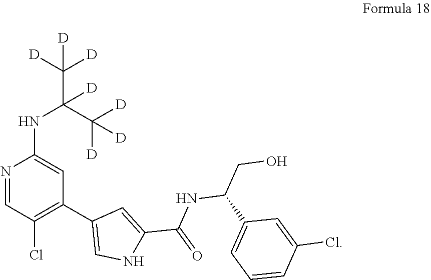 [SMILES: [2H]C([2H])([2H])C([2H])(Nc1cc(-c2c[nH]c(C(=O)N[C@H](CO)c3cccc(Cl)c3)c2)c(Cl)cn1)C([2H])([2H])[2H]]

38. The method of claim 31 further comprising the step of reacting Formula 18 with HCl to produce the compound of Formula 18A:

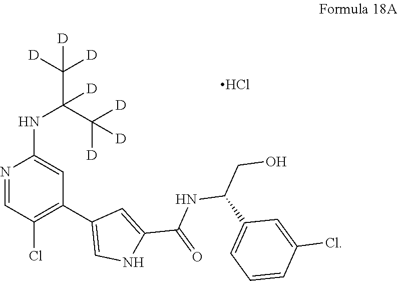 [SMILES: [2H]C([2H])([2H])C([2H])(Nc1cc(-c2c[nH]c(C(=O)N[C@H](CO)c3cccc(Cl)c3)c2)c(Cl)cn1)C([2H])([2H])[2H] ; Cl]

39. A method of synthesizing a deuterated ulixertinib of Formula 19, or a pharmaceutically acceptable salt thereof: 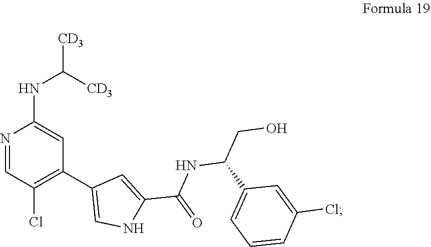 [SMILES: [2H]C([2H])([2H])C(Nc1cc(-c2c[nH]c(C(=O)N[C@H](CO)c3cccc(Cl)c3)c2)c(Cl)cn1)C([2H])([2H])[2H]]
comprising the steps of:
(i) reacting a compound of Formula 12A: 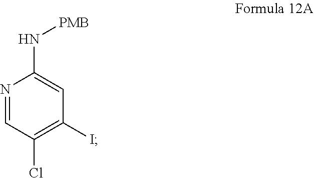
with a compound of Formula 8A: 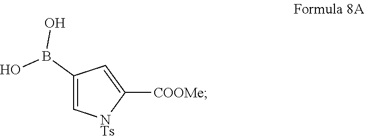 [SMILES: COC(=O)c1cc(B(O)O)cn1S(=O)(=O)c1ccc(C)cc1]
to produce a compound of Formula 13A: 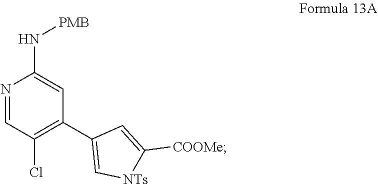
(ii) reacting the compound of Formula 13A with LiOH to produce the compound of Formula 14A: 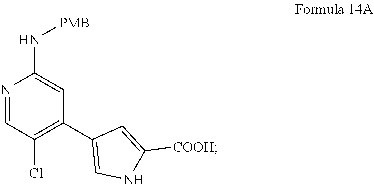
(iii) reacting the compound of Formula 14A with the compound of Formula 8B: 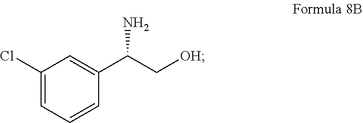 [SMILES: N[C@H](CO)c1cccc(Cl)c1]
to produce a compound of Formula 15A: 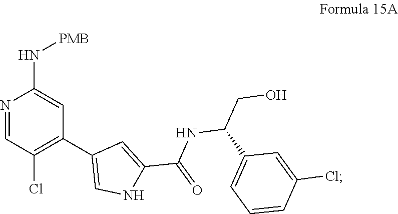
(iv) reacting the compound of Formula 15A with TFA to produce the compound of Formula 16A: 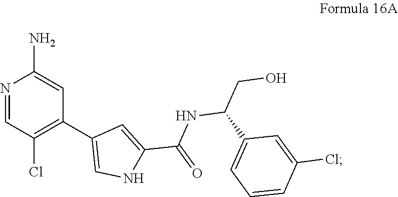 [SMILES: Nc1cc(-c2c[nH]c(C(=O)N[C@H](CO)c3cccc(Cl)c3)c2)c(Cl)cn1]
and
(v) reacting the compound of Formula 16A with 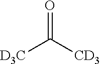
to produce the compound of Formula 19:

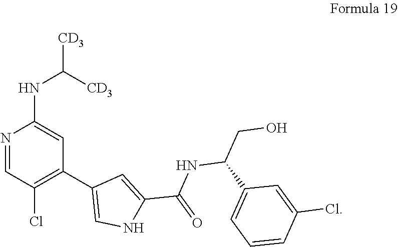 [SMILES: [2H]C([2H])([2H])C(Nc1cc(-c2c[nH]c(C(=O)N[C@H](CO)c3cccc(Cl)c3)c2)c(Cl)cn1)C([2H])([2H])[2H]]

40. The method of claim 39, further comprising the step of reacting Formula 19 with HCl to produce a compound of formula 19A

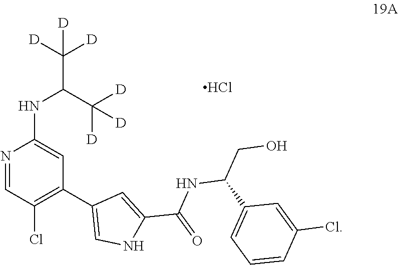 [SMILES: [2H]C([2H])([2H])C(Nc1cc(-c2c[nH]c(C(=O)N[C@H](CO)c3cccc(Cl)c3)c2)c(Cl)cn1)C([2H])([2H])[2H] ; Cl]

41. A method of synthesizing a deuterated ulixertinib of Formula 21, or a pharmaceutically acceptable salt thereof: 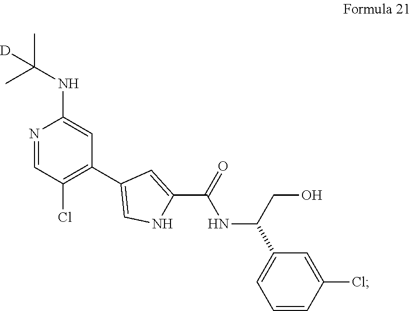 [SMILES: [2H]C(C)(C)Nc1cc(-c2c[nH]c(C(=O)N[C@H](CO)c3cccc(Cl)c3)c2)c(Cl)cn1]
comprising the steps of:
(i) reacting a compound of Formula 7B: 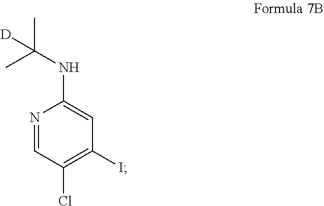
with a compound of Formula 8A: 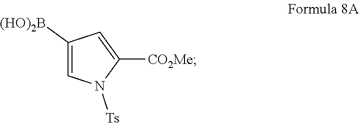 [SMILES: COC(=O)c1cc(B(O)O)cn1S(=O)(=O)c1ccc(C)cc1]
to produce a compound of Formula 9B: 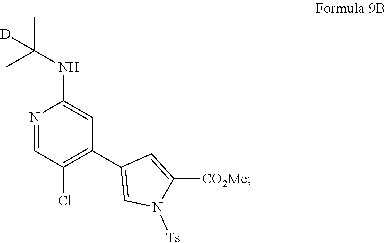 [SMILES: [2H]C(C)(C)Nc1cc(-c2cc(C(=O)OC)n(S(=O)(=O)c3ccc(C)cc3)c2)c(Cl)cn1]
(ii) reacting the compound of Formula 9B with LiOH to produce a compound of Formula 10B: 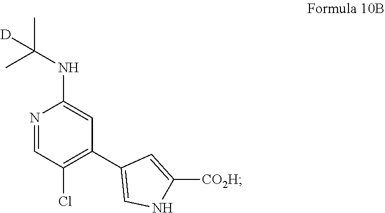 [SMILES: [2H]C(C)(C)Nc1cc(-c2c[nH]c(C(=O)O)c2)c(Cl)cn1]
and
(iii) reacting the compound of Formula 10B with the compound of Formula 8B: 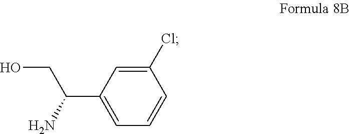 [SMILES: N[C@H](CO)c1cccc(Cl)c1]
to produce the compound of Formula 21:

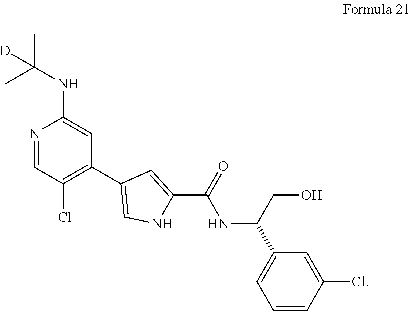 [SMILES: [2H]C(C)(C)Nc1cc(-c2c[nH]c(C(=O)N[C@H](CO)c3cccc(Cl)c3)c2)c(Cl)cn1]

42. A method of synthesizing a deuterated ulixertinib of Formula 21A comprising the step of reacting a compound of Formula 21: 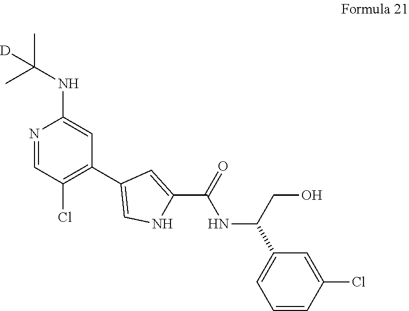 [SMILES: [2H]C(C)(C)Nc1cc(-c2c[nH]c(C(=O)N[C@H](CO)c3cccc(Cl)c3)c2)c(Cl)cn1]
with HCl to produce the compound of Formula 21A:

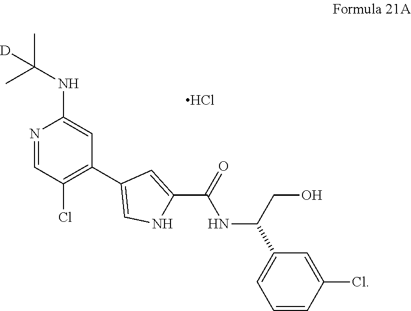 [SMILES: [2H]C(C)(C)Nc1cc(-c2c[nH]c(C(=O)N[C@H](CO)c3cccc(Cl)c3)c2)c(Cl)cn1 ; Cl]

43. A compound selected from: 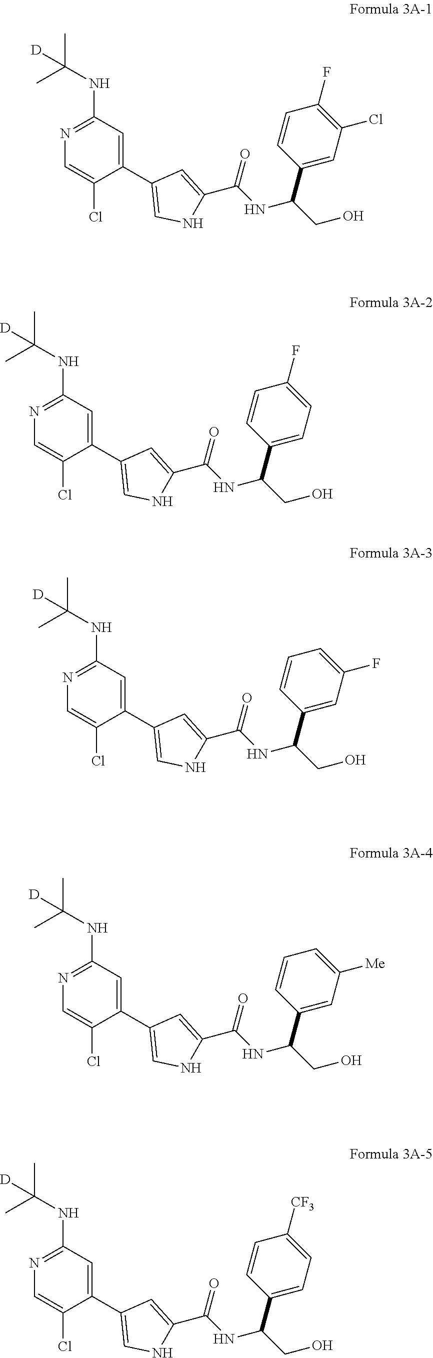 [SMILES: [2H]C(C)(C)Nc1cc(-c2c[nH]c(C(=O)N[C@H](CO)c3ccc(F)c(Cl)c3)c2)c(Cl)cn1 ; [2H]C(C)(C)Nc1cc(-c2c[nH]c(C(=O)N[C@H](CO)c3ccc(F)cc3)c2)c(Cl)cn1 ; [2H]C(C)(C)Nc1cc(-c2c[nH]c(C(=O)N[C@H](CO)c3cccc(F)c3)c2)c(Cl)cn1 ; [2H]C(C)(C)Nc1cc(-c2c[nH]c(C(=O)N[C@H](CO)c3cccc(C)c3)c2)c(Cl)cn1 ; [2H]C(C)(C)Nc1cc(-c2c[nH]c(C(=O)N[C@H](CO)c3ccc(C(F)(F)F)cc3)c2)c(Cl)cn1] 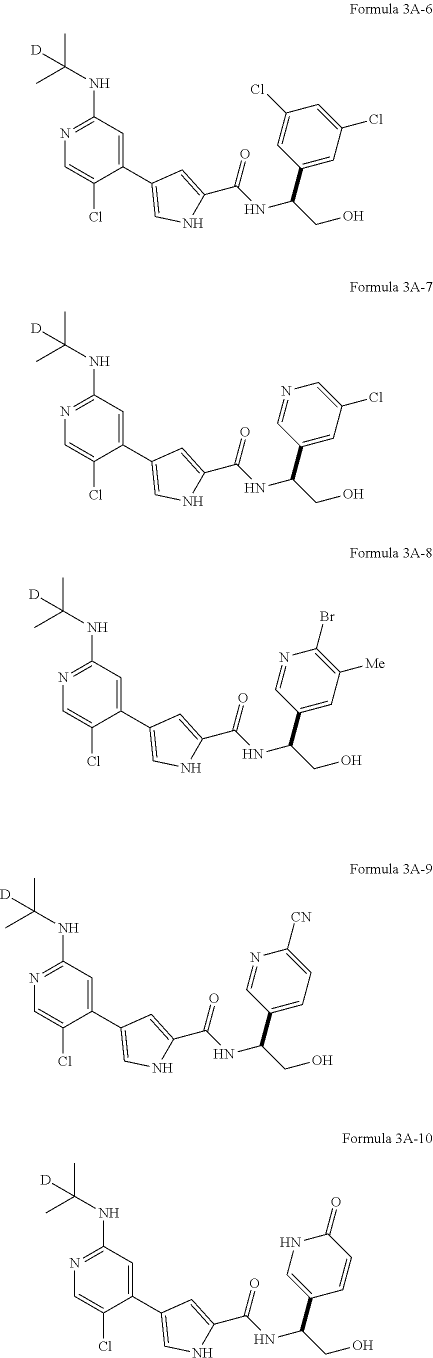 [SMILES: [2H]C(C)(C)Nc1cc(-c2c[nH]c(C(=O)N[C@H](CO)c3cc(Cl)cc(Cl)c3)c2)c(Cl)cn1 ; [2H]C(C)(C)Nc1cc(-c2c[nH]c(C(=O)N[C@H](CO)c3cncc(Cl)c3)c2)c(Cl)cn1 ; [2H]C(C)(C)Nc1cc(-c2c[nH]c(C(=O)N[C@H](CO)c3cnc(Br)c(C)c3)c2)c(Cl)cn1 ; [2H]C(C)(C)Nc1cc(-c2c[nH]c(C(=O)N[C@H](CO)c3ccc(C#N)nc3)c2)c(Cl)cn1 ; [2H]C(C)(C)Nc1cc(-c2c[nH]c(C(=O)N[C@H](CO)c3ccc(=O)[nH]c3)c2)c(Cl)cn1] 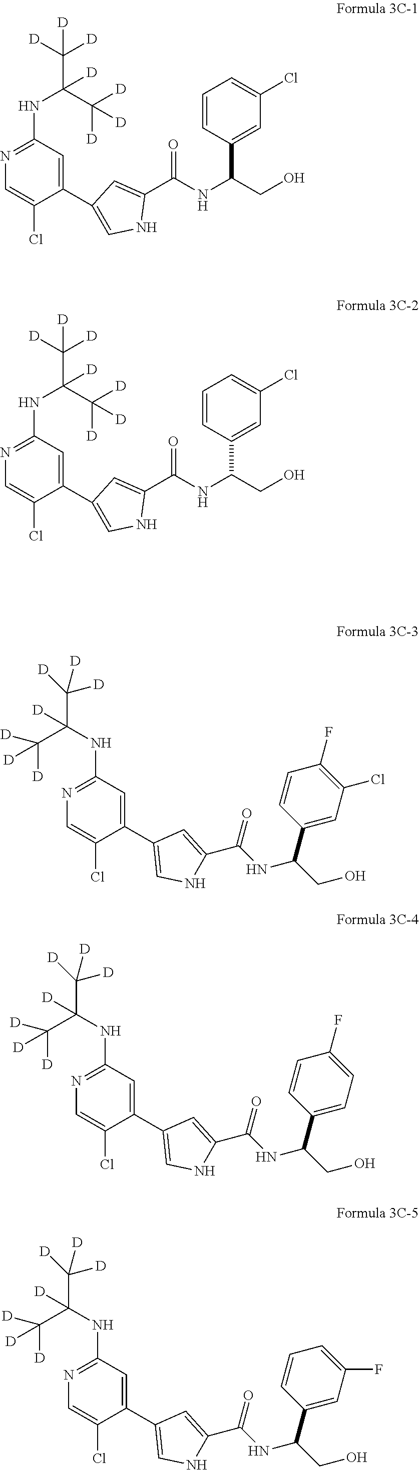 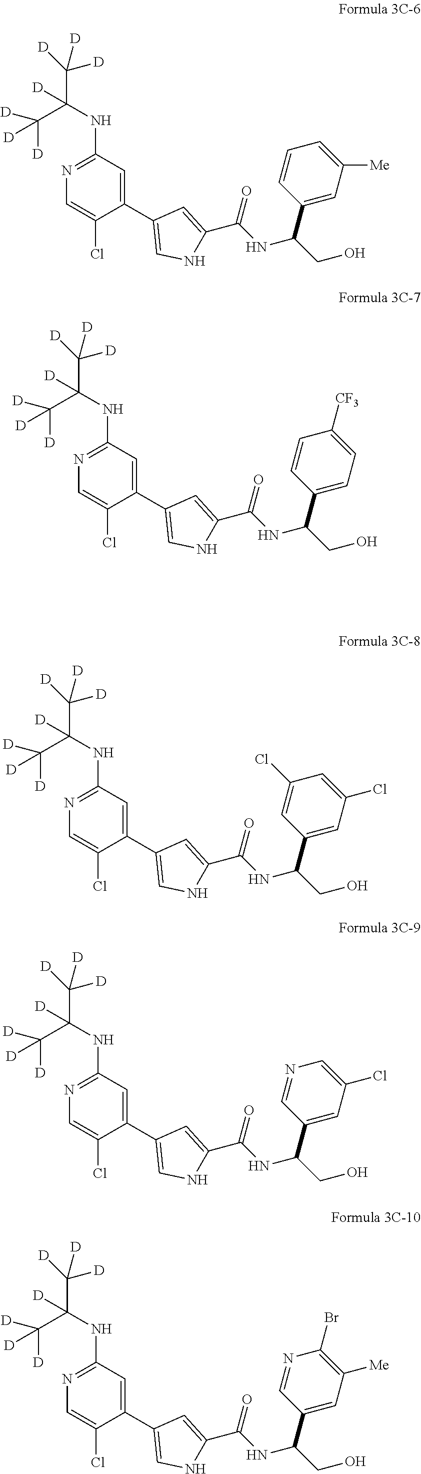 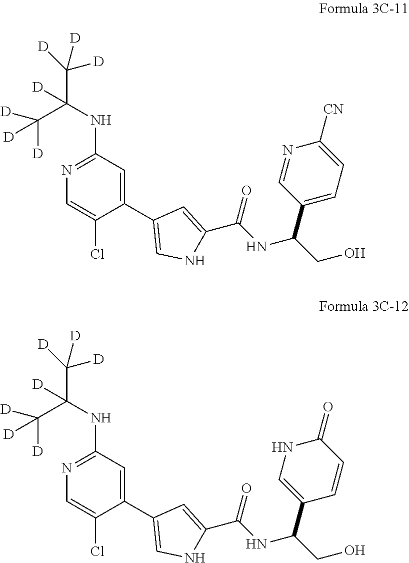 [SMILES: [2H]C([2H])([2H])C([2H])(Nc1cc(-c2c[nH]c(C(=O)N[C@H](CO)c3ccc(C#N)nc3)c2)c(Cl)cn1)C([2H])([2H])[2H] ; [2H]C([2H])([2H])C([2H])(Nc1cc(-c2c[nH]c(C(=O)N[C@H](CO)c3ccc(=O)[nH]c3)c2)c(Cl)cn1)C([2H])([2H])[2H]]
and pharmaceutically acceptable salts, solvates, and prodrugs thereof.

44. A kit for treating or ameliorating the effects of a disease in a subject, the kit comprising a compound according to claims 1-8 or a pharmaceutical composition according to claim 12 packaged together with instructions for its use.

45. The kit according to claim 44, wherein the compound or pharmaceutical composition is effective to decrease metabolism of the compound or pharmaceutical composition by at least one polymorphically-expressed cytochrome P₄₅₀ isoform as compared to the corresponding non-isotopically enriched compound.
